SEQUENCE LISTING (84 sequences)

SEQ ID NO: 1 - DNA, 28 bp, synthetic construct
  misc_feature 1..28: note = At4CL_FWD with NdeI Primer
  source 1..28: mol_type = other DNA
gcgccgcatatggcgccacaagaacaag

SEQ ID NO: 2 - DNA, 28 bp, synthetic construct
  misc_feature 1..28: note = At4CL_REV with XhoI Primer
  source 1..28: mol_type = other DNA
gcgcggctcgagtcacaatccatttgct

SEQ ID NO: 3 - DNA, 20 bp, synthetic construct
  misc_feature 1..20: note = Seq_T7_FWD Primer
  source 1..20: mol_type = other DNA
taatacgactcactataggg

SEQ ID NO: 4 - DNA, 19 bp, synthetic construct
  misc_feature 1..19: note = Seq_T7Term_REV Primer
  source 1..19: mol_type = other DNA
gctagttattgctcagcgg

SEQ ID NO: 5 - DNA, 50 bp, synthetic construct
  misc_feature 1..50: note = SDM_At4CL_NheI_FWD Primer
  source 1..50: mol_type = other DNA
gaatgacggaagcaggtccagtgctcgcaatgtcgttaggttttgcaaag

SEQ ID NO: 6 - DNA, 50 bp, synthetic construct
  misc_feature 1..50: note = SDM_At4CL_NheI_REV Primer
  source 1..50: mol_type = other DNA
ctttgcaaaacctaacgacattgcgagcactggacctgcttccgtcattc

SEQ ID NO: 7 - DNA, 65 bp, synthetic construct
  misc_feature 1..65: note = ANS_XbaI_F Primer
  source 1..65: mol_type = other DNA
ccctctagaaataattttgtttaactttaagaaggagatatacatatggtgaatgcagtagttac

SEQ ID NO: 8 - DNA, 34 bp, synthetic construct
  misc_feature 1..34: note = ANS_XhoI_R Primer
  source 1..34: mol_type = other DNA
cgatctcgagctatttagattcttcagcagcaac

SEQ ID NO: 9 - DNA, 26 bp, synthetic construct
  misc_feature 1..26: note = 3GT_NdeI_F Primer
  source 1..26: mol_type = other DNA
gcatcatatgaccaaaccctccgacc

SEQ ID NO: 10 - DNA, 35 bp, synthetic construct
  misc_feature 1..35: note = 3GT_XhoI_R Primer
  source 1..35: mol_type = other DNA
cgatctcgagtcaaataatgtttacaactgcatcc

SEQ ID NO: 11 - DNA, 25 bp, synthetic construct
  misc_feature 1..25: note = pETM6_ALL_inserts_flank_F Primer
  source 1..25: mol_type = other DNA
ccatcggtgatgtcggcgatatagg

SEQ ID NO: 12 - DNA, 27 bp, synthetic construct
  misc_feature 1..27: note = pETM6_ALL_inserts_flank_R Primer
  source 1..27: mol_type = other DNA
gtcgaggtgccgtaaagcactaaatcg

SEQ ID NO: 13 - DNA, 31 bp, synthetic construct
  misc_feature 1..31: note = ANS_mid_seq_F Primer
  source 1..31: mol_type = other DNA
ccatctggcctaaaaatcctactgactacac

SEQ ID NO: 14 - DNA, 29 bp, synthetic construct
  misc_feature 1..29: note = ANS_mid_seq_R Primer
  source 1..29: mol_type = other DNA
cctctttgaagactttgtgttcaacagcg

SEQ ID NO: 15 - DNA, 26 bp, synthetic construct
  misc_feature 1..26: note = 3GT_mid_seq_F Primer
  source 1..26: mol_type = other DNA
gcttcatcaaatgggtcttgctttgc

SEQ ID NO: 16 - DNA, 27 bp, synthetic construct
  misc_feature 1..27: note = 3GT_mid_seq_R Primer
  source 1..27: mol_type = other DNA
ggtgtcatgaccgtaccaaagctaatg

SEQ ID NO: 17 - DNA, 29 bp, synthetic construct
  misc_feature 1..29: note = RgTALsyn_FWD w/NdeI Primer
  source 1..29: mol_type = other DNA
gcggcgcatatggcgcctcgcccgacttc

SEQ ID NO: 18 - DNA, 40 bp, synthetic construct
  misc_feature 1..40: note = RgTALsyn_REV w/SpeI Primer
  source 1..40: mol_type = other DNA
gcggcgactagtttatgccagcatcttcagcagaacattg

SEQ ID NO: 19 - DNA, 36 bp, synthetic construct
  misc_feature 1..36: note = SDM_RgTALsyn_FWD Primer
  source 1..36: mol_type = other DNA
gcactgcacgacgcgcacatgttgagcctgttgagc

SEQ ID NO: 20 - DNA, 36 bp, synthetic construct
  misc_feature 1..36: note = SDM_RgTALsyn_REV Primer
  source 1..36: mol_type = other DNA
gctcaacaggctcaacatgtgcgcgtcgtgcagtgc

SEQ ID NO: 21 - DNA, 18 bp, synthetic construct
  misc_feature 1..18: note = pXylA_FOR Primer
  source 1..18: mol_type = other DNA
gcaagcatgcgaaatgca

SEQ ID NO: 22 - DNA, 19 bp, synthetic construct
  misc_feature 1..19: note = pXylA_REV Primer
  source 1..19: mol_type = other DNA
gagtttcgttcgagatcgc

SEQ ID NO: 23 - DNA, 312 bp, synthetic construct
  misc_feature 1..312: note = gBlock Sequence for cloning pXylA
  source 1..312: mol_type = other DNA
gcaagcatgcgaaatgcacctaggaaaaaaaacattgaaataaacatttattttgtatatgatgagataaagttagtttattggataaacaaactaactcaattaagatagttgatggataaacttgttcacttaaatcaaagggggaaatgtacacatatggcagatctcaattggatatcggccggccacgcgatcgctgacgtcggtaccctcgagtctggtaaagaaaccgctgctgcgaaatttgaacgccagcacatggactcgtctactagtcgcagcttaattaagcgatctcgaacgaaactc

SEQ ID NO: 24 - DNA, 1515 bp, Rhizobium trifolii
  source 1..1515: mol_type = genomic DNA
gtgagcaaccatcttttcgacgccatgcgggccgccgcgcccggtaacgcaccattcatccggatcgataacacgcgcacatggacctatgacgacgccgtcgctctttccggccgcattgccggcgcgatggacacgctcggcattcgccccggcgaccgcgttgcggtgcaggtcgagaaaagtgccgaggcattgatcctctatctcgcctgtcttcgaagcggcgccgtttacctgccgctcaacaccgcctatacgctggctgagctcgattattttatcggcgatgcggagccgcgtttggtggttgttgcatcgtcggctcgagcgggcgtggagacaatcgccaagccccgcggtgcgatcgtcgaaactctcgacgctgatggcagcggctcgttgctggatctcgcccgcgatgagccggctgactttgtcgatgcctcgcgctccgccgatgatctggctgcgatcctctacacctcgggaacgacgggacgctccaagggggcgatgctcacgcatgggaacctgctctcgaacgccctgaccttgcgagatttttggcgcgtcaccgccggcgatcgactgatccatgccttgccgatcttccacacgcatgggctgttcgtcgccacgaacgtcactttactcgccggcgcctcgatgttcctgctgtcgaagttcgacccggaggagatcctgtcgctgatgccgcaggcaacgatgctgatgggcgtgccgaccttctacgtgcgcctcctgcaaagcccgcgcctcgacaagcaagcagtcgccaacatccgcctcttcatttccggttcggctccactgcttgcagaaacacataccgagttccaggcacgtaccggtcacgccattctcgagcgctacggcatgacggaaaccaatatgaacacgtccaacccttatgaggggaaacggattgccggaacggtcggcttcccgctgcctgatgtgacggtgcgcgtcaccgatcccgccaccgggctcgcgctgccgcctgaagaaacaggcatgatcgagatcaaggggccgaacgttttcaagggctattggcgcatgcccgaaaaaaccgcggccgaattcaccgccgacggtttcttcatcagcggcgatctcggcaagatcgaccgggacggttatgtccacatcgtcggccgtggcaaggatctggtgatttccggtggatacaacatctatccgaaagaggtggagggcgagatcgaccagatcgagggtgtggttgagagcgctgtgatcggcgtgccgcatcccgatttcggagaaggcgtgaccgccgtcgtcgtgcgcaaacccggcgctgtcctcgatgaaaaggccatcgtcagcgccctccaggaccggctcgcgcgctacaaacaacccaagcgcatcatctttgccgaagacttgccgcgcaacacgatgggcaaggttcagaaaaacatcctgcggcagcaatacgccgatctttacaccaggacgtaa

SEQ ID NO: 25 - protein, 504 aa, Rhizobium trifolii
MSNHLFDAMRAAAPGNAPFIRIDNTRTWTYDDAVALSGRIAGAMDTLGIRPGDRVAVQVEKSAEALILYLACLRSGAVYLPLNTAYTLAELDYFIGDAEPRLVVVASSARAGVETIAKPRGAIVETLDADGSGSLLDLARDEPADFVDASRSADDLAAILYTSGTTGRSKGAMLTHGNLLSNALTLRDFWRVTAGDRLIHALPIFHTHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEETGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAVLDEKAIVSALQDRLARYKQPKRIIFAEDLPRNTMGKVQKNILRQQYADLYTRT

SEQ ID NO: 26 - DNA, 1347 bp, Rhizobium trifolii
  source 1..1347: mol_type = genomic DNA
atgggcatcgaactgctgagtattggtctgctgattgctatgtttattattgctacgattcaaccgattaacatgggtgctctggcattcgcaggcgcttttgtgctgggtagcatgattatcggcatgaaaaccaacgaaattttcgcaggctttccgtctgacctgtttctgaccctggtggcggttacgtacctgtttgcgattgcccagatcaatggcaccatcgactggctggttgaatgcgcggtgcgtctggttcgtggccgcattggtctgatcccgtgggtgatgttcctggttgcggccattatcaccggttttggtgcactgggtccggcagctgttgcaattctggcaccggtcgcactgagcttcgcagtgcaatatcgcattcatccggttatgatgggtctgatggtcatccacggcgcacaggctggcggtttttcaccgatttcgatctacggcggtattaccaaccaaatcgtggcaaaagcaggtctgccgttcgcaccgacgagtctgtttctgagcagctttttctttaatctggcaattgctgtcctggtgttctttgtgtttggcggtgcacgtgttatgaaacacgatccggcttctctgggtccgctgccggaactgcatccggaaggcgtgagcgcgtctattcgtggtcatggcggcaccccggcaaaaccgatccgcgaacatgcgtatggcaccgcagcagacacggcaaccacgctgcgtctgaacaatgaacgcattaccacgctgatcggtctgaccgcactgggtattggtgcactggttttcaaatttaacgtcggtctggtggctatgaccgtggcagtggttctggcactgctgagcccgaaaacgcagaaagcagctattgataaagtcagttggtccaccgtgctgctgatcgcgggtattatcacgtatgttggcgtcatggaaaaagcgggcaccgttgactacgtcgccaatggtattagttccctgggtatgccgctgctggtcgcgctgctgctgtgtttcaccggcgccatcgtgtccgcgtttgcctcatcgacggcactgctgggtgctattatcccgctggccgttccgttcctgctgcagggccatattagtgcaatcggtgtcgtggcggccattgctatctccaccacgattgtggataccagcccgttttctacgaacggcgcgctggttgtcgcaaatgctccggatgactcacgtgaacaggttctgcgccaactgctgatctattcggccctgattgctattattggtccgattgtcgcctggctggttttcgttgtgccgggtctggtctaa

SEQ ID NO: 27 - protein, 448 aa, Rhizobium trifolii
MGIELLSIGLLIAMFIIATIQPINMGALAFAGAFVLGSMIIGMKTNEIFAGFPSDLFLTLVAVTYLFAIAQINGTIDWLVECAVRLVRGRIGLIPWVMFLVAAIITGFGALGPAAVAILAPVALSFAVQYRIHPVMMGLMVIHGAQAGGFSPISIYGGITNQIVAKAGLPFAPTSLFLSSFFFNLAIAVLVFFVFGGARVMKHDPASLGPLPELHPEGVSASIRGHGGTPAKPIREHAYGTAADTATTLRLNNERITTLIGLTALGIGALVFKFNVGLVAMTVAVVLALLSPKTQKAAIDKVSWSTVLLIAGIITYVGVMEKAGTVDYVANGISSLGMPLLVALLLCFTGAIVSAFASSTALLGAIIPLAVPFLLQGHISAIGVVAAIAISTTIVDTSPFSTNGALVVANAPDDSREQVLRQLLIYSALIAIIGPIVAWLVFVVPGLV

SEQ ID NO: 28 - DNA, 2082 bp, Rhodotorula glutinis
  source 1..2082: mol_type = genomic DNA
atggcgcctcgcccgacttcgcaaagccaggcccgcacttgcccgacgacgcaggttacccaagttgatatcgttgagaaaatgttggcggctcctactgatagcacgctggagctggacggttatagcctgaatctgggtgatgtcgtgagcgctgcgcgtaagggtcgtcctgtccgtgtcaaagatagcgatgaaatccgcagcaaaatcgacaagagcgttgaattcctgcgcagccaactgagcatgtcggtttacggtgtgacgaccggctttggcggctccgcggacacgcgcacggaggacgcaattagcctgcaaaaggcgttgctggaacaccagctgtgtggtgtgttgccgagcagcttcgacagctttcgcttgggtcgtggtctggagaatagcctgccgttggaagtcgttcgcggtgcaatgaccattcgtgtgaattcgctgacccgtggccatagcgctgttcgtctggttgttctggaagcactgacgaactttctgaaccacggtattaccccgattgttccgctgcgcggtacgatctccgcgagcggcgatctgtctccactgtcgtacattgcagcggcgattagcggtcacccggatagcaaagttcacgtggtccatgaaggcaaagagaagatcctgtacgcgcgcgaagcgatggcgctgtttaacctggagccggtggttttgggtccgaaggagggcctgggtctggtgaatggtacggcagtctccgcgagcatggcaacgctggcactgcacgacgcgcatatgttgagcctgttgagccaatcgctgaccgcgatgaccgtggaggcgatggtcggtcacgcgggcagcttccatccattcctgcacgatgttacgcgtccgcacccgacgcaaatcgaggtcgcgggtaacattcgcaaactgctggagggctcgcgcttcgcggtccaccacgaggaagaggttaaggtcaaggatgatgaaggcattttgcgtcaggatcgttatccgttgcgcacgagcccgcaatggttgggtccgctggtgtccgacctgattcacgctcatgccgtcttgacgatcgaagcgggtcaaagcaccaccgataacccactgatcgatgttgagaataagaccagccatcacggtggcaactttcaagcggcagcggttgccaacacgatggaaaagacccgtctgggcttggcccaaatcggtaaactgaatttcacccagctgacggagatgctgaacgcgggcatgaatcgtggcttgccgagctgcctggcggctgaagacccatccctgagctatcattgcaaaggtctggacattgcggcggctgcatatacgagcgaactgggccacctggctaacccggtcaccacccacgtccaaccggctgaaatggcaaaccaggcggtgaatagcttggcgttgattagcgcacgtcgtaccacggaatctaacgacgttctgtccctgctgctggcaacgcacctgtactgcgtgctgcaggcgatcgacctgcgtgcgattgagttcgagttcaagaaacagtttggtcctgccattgttagcctgatcgaccaacactttggtagcgcgatgacgggtagcaatctgcgtgatgagctggttgaaaaggtcaataagactctggccaagcgtttggagcaaaccaatagctacgatctggttccgcgctggcacgacgcttttagcttcgctgcaggcactgttgtcgaggttctgtccagcacgagcctgagcttggcggccgtgaacgcatggaaggttgcggcagccgagagcgcgatctccttgacgcgccaggtccgtgaaacgttttggtccgctgcaagcacctccagcccggcgttgtcttacttgagcccgcgcacgcagatcctgtacgcatttgtgcgtgaggaactgggtgtcaaagcccgccgtggtgacgtcttcttgggtaaacaagaagttaccatcggcagcaacgttagcaagatttacgaagccatcaagagcggccgtatcaacaatgttctgctgaagatgctggcataa

SEQ ID NO: 29 - protein, 693 aa, Rhodotorula glutinis
MAPRPTSQSQARTCPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 30 - DNA, 1686 bp, Arabidopsis thaliana
  source 1..1686: mol_type = genomic DNA
atggcgccacaagaacaagcagtttctcaggtgatggagaaacagagcaacaacaacaacagtgacgtcattttccgatcaaagttaccggatatttacatcccgaaccacctatctctccacgactacatcttccaaaacatctccgaattcgccactaagccttgcctaatcaacggaccaaccggccacgtgtacacttactccgacgtccacgtcatctcccgccaaatcgccgccaattttcacaaactcggcgttaaccaaaacgacgtcgtcatgctcctcctcccaaactgtcccgaattcgtcctctctttcctcgccgcctccttccgcggcgcaaccgccaccgccgcaaaccctttcttcactccggcggagatagctaaacaagccaaagcctccaacaccaaactcataatcaccgaagctcgttacgtcgacaaaatcaaaccacttcaaaacgacgacggagtagtcatcgtctgcatcgacgacaacgaatccgtgccaatccctgaaggctgcctccgcttcaccgagttgactcagtcgacaaccgaggcatcagaagtcatcgactcggtggagatttcaccggacgacgtggtggcactaccttactcctctggcacgacgggattaccaaaaggagtgatgctgactcacaagggactagtcacgagcgttgctcagcaagtcgacggcgagaacccgaatctttatttccacagcgatgacgtcatactctgtgttttgcccatgtttcatatctacgctttgaactcgatcatgttgtgtggtcttagagttggtgcggcgattctgataatgccgaagtttgagatcaatctgctattggagctgatccagaggtgtaaagtgacggtggctccgatggttccgccgattgtgttggccattgcgaagtcttcggagacggagaagtatgatttgagctcgataagagtggtgaaatctggtgctgctcctcttggtaaagaacttgaagatgccgttaatgccaagtttcctaatgccaaactcggtcagggatacggaatgacggaagcaggtccagtgctcgcaatgtcgttaggttttgcaaaggaaccttttccggttaagtcaggagcttgtggtactgttgtaagaaatgctgagatgaaaatagttgatccagacaccggagattctctttcgaggaatcaacccggtgagatttgtattcgtggtcaccagatcatgaaaggttacctcaacaatccggcagctacagcagagaccattgataaagacggttggcttcatactggagatattggattgatcgatgacgatgacgagcttttcatcgttgatcgattgaaagaacttatcaagtataaaggttttcaggtagctccggctgagctagaggctttgctcatcggtcatcctgacattactgatgttgctgttgtcgcaatgaaagaagaagcagctggtgaagttcctgttgcatttgtggtgaaatcgaaggattcggagttatcagaagatgatgtgaagcaattcgtgtcgaaacaggttgtgttttacaagagaatcaacaaagtgttcttcactgaatccattcctaaagctccatcagggaagatattgaggaaagatctgagggcaaaactagcaaatggattgtga

SEQ ID NO: 31 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 32 - DNA, 1635 bp, Petroselinum crispum
  source 1..1635: mol_type = genomic DNA
atgggagactgtgtagcacccaaagaagaccttattttccgatcgaaactccctgatatttacatcccgaaacaccttccgttacatacttattgtttcgaaaacatctcgaaagttggcgacaagtcctgtttaataaatggcgctacaggcgaaacgttcacttattcccaagttgagctcctttccaggaaagttgcatcagggttaaacaaactcggcattcaacagggcgataccatcatgcttttgctccctaactcccctgagtattttttcgctttcttaggcgcatcgtatcgtggtgcaatttctactatggccaatccgtttttcacttctgctgaggtgatcaaacagctcaaagcatcccaagctaagctcataattacgcaagcttgttacgtagacaaagtgaaagactacgcagcagagaaaaatatacagatcatttgcatcgatgatgctcctcaggattgtttacatttctccaaacttatggaagctgatgaatcagaaatgcctgaggttgtgatcaattcagacgatgtcgtcgcgttaccttactcatcgggtactacaggactaccgaaaggtgttatgttgacacacaaaggacttgttactagcgtggcacaacaagttgatggagacaatccgaatttatatatgcatagcgaggatgtgatgatctgcatattgcctttgtttcatatttattcgcttaacgcggtgttgtgctgtggactcagagcaggggtgacgatcttgattatgcagaaatttgatattgtgccatttttggaactgatacagaaatataaagttacaattggaccgtttgtgccaccaattgtgttggcaattgcgaaaagtccagtggtggataaatatgacttgtcgtcggtgaggacggttatgtctggagctgctccgttagggaaggagcttgaagatgctgttagagctaagtttcctaatgccaaacttggtcagggatatggaatgacagaggcagggccagttttagcaatgtgcctggcgtttgcaaaggaaccatacgagatcaaatcgggtgcctgtggaactgttgtgaggaatgctgaaatgaaaattgtggatcctgagaccaacgcctctcttccacgaaaccaacgcggagagatttgcattcgaggtgaccaaattatgaaaggctacctcaatgatcctgaatcaacaaggacaacaatagacgaagaaggctggttgcacacaggagatataggcttcattgacgacgatgatgagctatttattgttgatagacttaaggaaataatcaaatacaaaggcttccaggttgcccctgctgaacttgaagctctgctacttactcatcctaccatttccgatgctgcagttgttcccatgatagatgagaaagcaggagaggtgcctgtggcttttgttgtgagaacaaacggtttcaccaccactgaggaagaaatcaagcaattcgtctcgaaacaggtggtgttctacaagagaatatttcgtgtattttttgttgatgcaattccgaaatcaccatctggaaagattcttcgaaaggacttgagagcaaaaatagcatccggtgatcttcccaaataa

SEQ ID NO: 33 - protein, 544 aa, Petroselinum crispum
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASQAKLIITQACYVDKVKDYAAEKNIQIICIDDAPQDCLHFSKLMEADESEMPEVVINSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRARIASGDLPK

SEQ ID NO: 34 - DNA, 1710 bp, Vitis vinifera
  source 1..1710: mol_type = genomic DNA
atgattagtattgaaacgcaaaacccggatgttagcaacctggacacctcgcactctattccgaaaatggcaaaccgtattgatgaccatgtgtttcgttctaaactgccggaaattccgatcagtaaccatctgccgctgcacacgtattgcttcgaaaattactcgcagtttgcagaccgtccgtgtctgattgttggctcgacgaacaaaacctatagcttcgctgaaacccatctgatctctcgcaaagtgggcgcaggttttgctcacctgggtctgaaacagggcgatgtggttatgattctgctgcaaaattgcgcggaatttgccttcagctttctgggtgcgtctatggttggcgccgtcaccacgaccgcaaacccgttctacacgtccgcggaaatcttcaaacagctgaacgcatcaaaagctaaaatcgtcgtgacccaggcgcaatatgtggataaactgcgcgactacccggatggtcaagttgccaaaattggcgaaggtttcacggtcattaccatcgatgacccgccggaaaactgtatgcattttagtgttgtctccgaagcgaacgaaagcgaactgccggaagtctcaattaattcggatgacccggtggccctgccgtttagctctggtacgaccggcctgccgaaaggcgtggttctgacgcacaaatcactgatcacctcggtcgcccagcaagtggatggtgaaaacccgaatctgcatctgaccccggatgacgtcgtgctgtgcgtgctgccgctgttccacatttatagcctgaactctgttctgctgtgtagtctgcgtgcaggtgcagcagtgctgctgatgcagaaatttgaaattggtaccctgctggaactgatccaacgttaccgcgtgagcgttgcagctgttgtcccgccgctggttctggcactggctaaaaatccgatggtggaatcgtttgatctgagttccatccgtgtggttctgagcggtgcagcaccgctgggcaaagaactggaagcagctctgcgttcccgcgttccgcaggcagtcctgggccaaggttatggcatgacggaagcaggcccggtgctgtcaatgtgcctgggtttcgctaaacagccgtttccgacgaaatcaggttcgtgtggcaccgtcgtgcgtaacgcggaactgaaagttgtggatccggaaaccggttgctccctgggccgtaatcagccgggtgaaatttgtatccgcggccagcaaattatgaaaggttatctgaatgatccggaagcgacggcctctaccattgacgttgatggctggctgcataccggtgacatcggctacgtggatgacgatgaagaagtgttcattgttgatcgcgtcaaagaactgatcaaattcaaaggttttcaggttccgccggcagaactggaagctctgctggtgtctcacccgtccattgccgatgcggccgtggttccgcaaaaagacgatgttgctggcgaagtcccggtggcgttcgtcgtgcgttctaacggttttgaactgaccgaagaagcagtgaaagaattcatcagtaaacaggttgtcttttataaacgcctgcataaagtgtactttgttcacgcgattccgaaaagcccgtctggcaaaatcctgcgtaaagatctgcgcgcgaaactggccgaaaaaaccccggaaccgaac

SEQ ID NO: 35 - protein, 570 aa, Vitis vinifera
MISIETQNPDVSNLDTSHSIPKMANRIDDHVFRSKLPEIPISNHLPLHTYCFENYSQFADRPCLIVGSTNKTYSFAETHLISRKVGAGFAHLGLKQGDVVMILLQNCAEFAFSFLGASMVGAVTTTANPFYTSAEIFKQLNASKAKIVVTQAQYVDKLRDYPDGQVAKIGEGFTVITIDDPPENCMHFSVVSEANESELPEVSINSDDPVALPFSSGTTGLPKGVVLTHKSLITSVAQQVDGENPNLHLTPDDVVLCVLPLFHIYSLNSVLLCSLRAGAAVLLMQKFEIGTLLELIQRYRVSVAAVVPPLVLALAKNPMVESFDLSSIRVVLSGAAPLGKELEAALRSRVPQAVLGQGYGMTEAGPVLSMCLGFAKQPFPTKSGSCGTVVRNAELKVVDPETGCSLGRNQPGEICIRGQQIMKGYLNDPEATASTIDVDGWLHTGDIGYVDDDEEVFIVDRVKELIKFKGFQVPPAELEALLVSHPSIADAAVVPQKDDVAGEVPVAFVVRSNGFELTEEAVKEFISKQVVFYKRLHKVYFVHAIPKSPSGKILRKDLRAKLAEKTPEPN

SEQ ID NO: 36 - DNA, 1170 bp, Petunia x hybrida
  source 1..1170: mol_type = genomic DNA
atggtgacagtcgaggagtatcgtaaggcacaacgtgctgaaggtccagccactgtcatggccattggaacagccacaccttcaaactgtgttgatcaaagcacttaccctgatttttattttcgtatcactaacagtgagcacaagactgatcttaaggagaaatttaagcgcatgtgtgaaaaatcaatgattaagaaaaggtacatgcacttaacagaggaaatcttgaaagagaatcctagtatgtgtgaatacatggcaccttctcttgatgctaggcaagacatagtggtggttgaagtgcccaaacttggcaaagaggcagctcaaaaggccatcaaggaatggggccagcccaagtccaaaattacccatttggtcttttgcacaaccagtggtgtggacatgcctgggtgtgactatcaactcactaagctacttgggcttcgtccatcggtcaagaggcttatgatgtaccaacaaggttgctttgctggtggcacggttcttcggttagccaaggacttggctgaaaacaacaagggcgctcgagtccttgttgtttgttcagaaatcaccgcggtcactttccgtgggccaaatgatactcatttggatagtttagttggccaagcactttttggtgatggggcaggcgcgatcattataggttctgatccaattccaggggtcgaaaggcctttgttcgagctcgtttcagcagcccaaactcttctcccagatagccatggtgctattgatggccatctccgtgaagttgggcttacattccacttactcaaagatgttcctgggctgatctcaaaaaatattgagaagagccttgaggaagcattcaaacctttgggcatttctgattggaactctctattctggattgctcatccaggtgggcctgcaattttggaccaagttgaaataaagttgggcctaaagcccgagaaacttaaggctacaaggaatgtgttaagtaactatggtaacatgtcaagtgcttgtgtactgtttattttggatgaaatgagaaaggcctcagccaaagaaggtttaggaactactggtgaagggcttgagtggggtgttctttttggatttgggcctgggctaacagttgagactgttgtcctccacagtgttgctacttaa

SEQ ID NO: 37 - protein, 389 aa, Petunia x hybrida
MVTVEEYRKAQRAEGPATVMAIGTATPSNCVDQSTYPDFYFRITNSEHKTDLKEKFKRMCEKSMIKKRYMHLTEEILKENPSMCEYMAPSLDARQDIVVVEVPKLGKEAAQKAIKEWGQPKSKITHLVFCTTSGVDMPGCDYQLTKLLGLRPSVKRLMMYQQGCFAGGTVLRLAKDLAENNKGARVLVVCSEITAVTFRGPNDTHLDSLVGQALFGDGAGAIIIGSDPIPGVERPLFELVSAAQTLLPDSHGAIDGHLREVGLTFHLLKDVPGLISKNIEKSLEEAFKPLGISDWNSLFWIAHPGGPAILDQVEIKLGLKPEKLKATRNVLSNYGNMSSACVLFILDEMRKASAKEGLGTTGEGLEWGVLFGFGPGLTVETVVLHSVAT

SEQ ID NO: 38 - DNA, 1254 bp, Citrus maxima
  source 1..1254: mol_type = genomic DNA
atggctacggtccaagaaatccgcaacgctcaacgcgcagatggtccggcgacggtcctggcaatcggcacggcaaccccggctcatagcgtgaaccaggcagattatccggactattactttcgtattaccaaatctgaacacatgacggaactgaaagaaaaattcaaacgtatgtgcgataaaagtatgattaaaaaacgctacatgtacctgaccgaagaaatcctgaaagaaaacccgaatatgtgtgcctacatggcaccgagcctggatgcgcgccaggacattgtggttgtcgaagttccgaaactgggtaaagaagcggccaccaaagccatcaaagaatggggccaaccgaaatcaaaaattacgcacctgatcttttgcaccacgtcgggtgtggatatgccgggtgcagactatcagctgaccaaactgctgggtctgcgtccgagcgttaaacgctttatgatgtaccagcaaggctgcttcgcaggcggtacggtcctgcgtctggctaaagatctggcggaaaacaataaaggtgctcgcgttctggtggtttgtagtgaaattaccgctgtcacgtttcgtggtccggcggatacccatctggactccctggttggccaggccctgttcggcgatggtgcagctgcggttatcgtcggcgcagatccggacacgagtgtggaacgtccgctgtatcagctggtttcaacctcgcaaacgattctgccggattccgacggtgcgatcgatggccatctgcgcgaagtgggtctgacctttcacctgctgaaagacgttccgggcctgatttcaaaaaacatcgaaaaaagcctgtctgaagcctttgcaccggttggtatttcggattggagctctattttctggatcgcacatccgggcggtccggcaatcctggaccaggtggaaagcaaactgggtctgaaagaagaaaaactgaaagctacccgtcaagtcctgtctgaatacggcaatatgagttccgcgtgtgtgctgttcattctggatgaaatgcgcaaaaaatctgccgaagaagctaaagcgaccacgggcgaaggtctggattggggcgtgctgtttggtttcggtccgggtctgaccgtcgaaacggtcgtgctgcacagtgtgccgatcaaagcgggcggtggcggttccggcggtggtggtagtggtggtggtggctctccgccgccggccctgccgccgaaacgtcgtcgctaa

SEQ ID NO: 39 - protein, 417 aa, Citrus maxima
MATVQEIRNAQRADGPATVLAIGTATPAHSVNQADYPDYYFRITKSEHMTELKEKFKRMCDKSMIKKRYMYLTEEILKENPNMCAYMAPSLDARQDIVVVEVPKLGKEAATKAIKEWGQPKSKITHLIFCTTSGVDMPGADYQLTKLLGLRPSVKRFMMYQQGCFAGGTVLRLAKDLAENNKGARVLVVCSEITAVTFRGPADTHLDSLVGQALFGDGAAAVIVGADPDTSVERPLYQLVSTSQTILPDSDGAIDGHLREVGLTFHLLKDVPGLISKNIEKSLSEAFAPVGISDWSSIFWIAHPGGPAILDQVESKLGLKEEKLKATRQVLSEYGNMSSACVLFILDEMRKKSAEEAKATTGEGLDWGVLFGFGPGLTVETVVLHSVPIKAGGGGSGGGGSGGGGSPPPALPPKRRR

SEQ ID NO: 40 - DNA, 735 bp, Citrus maxima
  source 1..735: mol_type = genomic DNA
atgaatccgtcgccgtctgttaccgaactgcaagtggaaaatgtcacctttacgccgagtctgcaaccgccgggctctaccaaatcgcattttctgggcggtgcaggtgaacgtggcctggaaatcgaaggcaaatttgttaaattcaccgctattggtgtctatctggaagaaaacgccgtgccgctgctggcaggcaaatggaaaggcaaaaccgccggtgaactgacggaatctgtcgaatttttccgcgatgtggttaccggcccgtttgaaaaattcatgaaagtgaccatgatcctgccgctgacgggtgcgcagtattcagaaaaagttgctgaaaattgcatggcgatttggaaatttttcggcatctacaccgatgcagaagctaaagcgattgaaaaatttacggaagtgttcaaagacgaaatttttccgccgggcagctctatcctgttcacccaaagttccggttcgctgacgatttcattttcgaaagatggcagcatcccgaaagacggtgtcgcggtgattgaaaacaatctgctgagcgaagccgttctggaatctatgatcggtaaaaacggcgtcagtccggcggccaaaaaatccctggccgaacgtctgtcagcactgctgaatgttgcttccgacaaaatgaaaggcggtggcggctcaggtggcggtggctctggtggcggtggttcaggcgtcaaagaaagtctggtgtga

SEQ ID NO: 41 - protein, 244 aa, Citrus maxima
MNPSPSVTELQVENVTFTPSLQPPGSTKSHFLGGAGERGLEIEGKFVKFTAIGVYLEENAVPLLAGKWKGKTAGELTESVEFFRDVVTGPFEKFMKVTMILPLTGAQYSEKVAENCMAIWKFFGIYTDAEAKAIEKFTEVFKDEIFPPGSSILFTQSSGSLTISFSKDGSIPKDGVAVIENNLLSEAVLESMIGKNGVSPAAKKSLAERLSALLNVASDKMKGGGGSGGGGSGGGGSGVKESLV

SEQ ID NO: 42 - DNA, 669 bp, Medicago sativa
  source 1..669: mol_type = genomic DNA
atggctgcatcaatcaccgcaatcactgtggagaaccttgaatacccagcggtggttacctctccggtcaccggcaaatcatatttcctcggtggcgctggggagagaggattgaccattgaaggaaacttcatcaagttcactgccataggtgtttatttggaagatatagcagtggcttcactagctgccaaatggaagggtaaatcatctgaagagttacttgagacccttgacttttacagagacatcatctcaggtccctttgaaaagttaattagagggtcaaagattagggaattgagtggtcctgagtactcaaggaaggttatggagaactgtgtggcacacttgaaatcagttggaacttatggagatgcagaagctgaagctatgcaaaaatttgctgaagctttcaagcctgttaattttccacctggtgcctctgttttctacaggcaatcacctaatggaatattagggcttagtttctctccggatacaagtataccagaaaaggaggctgcactcatagagaacaaggcagtttcatcagcagtgttggagactatgatcggcgagcacgctgtttcccctgatcttaagcgctgtttagctgcaagattacctgcgttgttgaacgagggtgctttcaagattggaaactga

SEQ ID NO: 43 - protein, 222 aa, Medicago sativa
MAASITAITVENLEYPAVVTSPVTGKSYFLGGAGERGLTIEGNFIKFTAIGVYLEDIAVASLAAKWKGKSSEELLETLDFYRDIISGPFEKLIRGSKIRELSGPEYSRKVMENCVAHLKSVGTYGDAEAEAMQKFAEAFKPVNFPPGASVFYRQSPNGILGLSFSPDTSIPEKEAALIENKAVSSAVLETMIGEHAVSPDLKRCLAARLPALLNEGAFKIGN

SEQ ID NO: 44 - DNA, 1200 bp, Camellia sinensis
  source 1..1200: mol_type = genomic DNA
atggcaccgaccaccaccctgaccgcactggcagaagaaaaaagcctgcagcagaaatttgttcgtgatgaagatgaacgtccgaaagttgcctataatgtgtttagcaatgaaatcccggttattagcctggcaggtattgatgaaattgaaggtcgtcgtagcgaaatttgccgtaaaattgttgaagcatgtgaaggttggggtgtttttcaggttgttgatcatggtgttgatgcaaatctgattgcagaaatgacccgtctggcacgtgaattttttgcactgcctccggaagaaaaactgcgttttgatatgagcggtggtaaaaaaggtggttttattgttagcagccatctgcagggtgaagcagttcaggattggcgtgaaattgttacctatttcagctatccgattcgtgcacgtgattatagccgttggcctgataaaccggaaggttggcgtgcagttaccgaaacctatagcgaaaaactgatggatctggcatgtaaactgctggaagttctgagcgaagcaatgggtctggaaaaagaggcactgaccaaagcatgtgttgatatggatcagaaagtggtgatcaacttctatccgaaatgtccgcagccggatctgaccctgggtctgaaacgtcataccgatccgggtacaattaccctgctgctgcaagatcaggtgggtggtctgcaggcaacccgtgatggtggcaaaacctggattaccgttcagccggttgaaggtgcatttgttgttaatctgggtgatcatggccattatctgagcaatggtcgctttaaaaacgcagatcatcaggcagttgttaatagcaattgtagccgtctgagcattgcaacctttcagaatccggcaccggaagcaaccgtttatccgctgaaaattcgtgaaggtgaaaaaccgattctggaagaaccgattacctttgccgatatgtataaacgcaaaatgagcaaagatatcgagctggccaaactgaaaaaactggcgaaagaaaaaaaactgctgcaagaccagcaggatatcgaaaaagcaaaactggaaatcaaaagcaccgatgaaatcttcgccctggttggtgcactgatgcatgttatgcagaaacgtagccgtgcaattcatagcagtgatgaaggtgaagatcaagccggtgatgaagatgaggat

SEQ ID NO: 45 - protein, 400 aa, Camellia sinensis
MAPTTTLTALAEEKSLQQKFVRDEDERPKVAYNVFSNEIPVISLAGIDEIEGRRSEICRKIVEACEGWGVFQVVDHGVDANLIAEMTRLAREFFALPPEEKLRFDMSGGKKGGFIVSSHLQGEAVQDWREIVTYFSYPIRARDYSRWPDKPEGWRAVTETYSEKLMDLACKLLEVLSEAMGLEKEALTKACVDMDQKVVINFYPKCPQPDLTLGLKRHTDPGTITLLLQDQVGGLQATRDGGKTWITVQPVEGAFVVNLGDHGHYLSNGRFKNADHQAVVNSNCSRLSIATFQNPAPEATVYPLKIREGEKPILEEPITFADMYKRKMSKDIELAKLKKLAKEKKLLQDQQDIEKAKLEIKSTDEIFALVGALMHVMQKRSRAIHSSDEGEDQAGDEDED

SEQ ID NO: 46 - DNA, 1191 bp, Malus domestica
  source 1..1191: mol_type = genomic DNA
atggcaccgcctgcaaccaccctgaccagcattgcacatgaaaaaaccctgcagcagaaatttgttcgtgatgaagatgaacgtccgaaagtggcctataatgaatttagcaacgaaatcccgattattagcctggcaggtattgatgaagttgaaggtcgtcgtgccgaaatctgcaaaaaaatcgttgaagcatgtgaagattggggcatttttcagattgttgatcatggtgttgatgccgaactgattagcgaaatgaccggtctggcaaaagaattttttgatctgccgagcgaagaaaaactgcgttttgatatgagcggtggtaaaaaaggtggttttattgttagcagccatctgcagggtgaagcagttcaggattggcgtgaaattgttacctattttctgtatccgattcgccaccgtgattatagccgttggcctgataaaccggaagcatggcgtgaagttaccaaaaaatacagtgatgaactgatgggtctggcatgtaaactgctgggtgttctgagcgaagcaatgggcctggataccgaagcactgaccaaagcatgtgttgatatggatcagaaagtggtggttaacttctatccgaaatgtccgcagccggatctgaccctgggtctgaaacgtcataccgatccgggtacaattaccctgctgctgcaagatcaggttggcggtctgcaggcaacccgtgatgatggtaaaacctggattaccgttcagccggttgaaggtgcatttgttgttaatctgggtgatcatggccattttctgagcaatggtcgctttaaaaacgcagatcatcaggcagttgttaatagcaatagcagccgtctgagcattgcaacctttcagaatccggcacaggatgcaattgtttatccgctgagcgttcgtgaaggtgaaaaaccgattctggaagcaccgattacctataccgagatgtataaaaaaaaaatgagcaaagatctggaactggcacgcctgaaaaaactggccaaagaacagcagctgcaggatctggaaaaagcaaaagttgaaaccaaaccggcagatgatatctttgccctggttggtgcactgatgcatgttatgcagaaacgtagccgtgcaattcatagcagtgatgaaggtgaagatcaagccggtgatgaagatgaggat

SEQ ID NO: 47 - protein, 397 aa, Malus domestica
MAPPATTLTSIAHEKTLQQKFVRDEDERPKVAYNEFSNEIPIISLAGIDEVEGRRAEICKKIVEACEDWGIFQIVDHGVDAELISEMTGLAKEFFDLPSEEKLRFDMSGGKKGGFIVSSHLQGEAVQDWREIVTYFLYPIRHRDYSRWPDKPEAWREVTKKYSDELMGLACKLLGVLSEAMGLDTEALTKACVDMDQKVVVNFYPKCPQPDLTLGLKRHTDPGTITLLLQDQVGGLQATRDDGKTWITVQPVEGAFVVNLGDHGHFLSNGRFKNADHQAVVNSNSSRLSIATFQNPAQDAIVYPLSVREGEKPILEAPITYTEMYKKKMSKDLELARLKKLAKEQQLQDLEKAKVETKPADDIFALVGALMHVMQKRSRAIHSSDEGEDQAGDEDED

SEQ ID NO: 48 - DNA, 1200 bp, Petroselinum crispum
  source 1..1200: mol_type = genomic DNA
atggcaccgagcaccctgaccgcactggcacaagaaaaaaccctgaatagcaaatttgtgcgcgacgaagatgaacgtccgaaaattgcatataacaaattcagcgacgaaatcccggttattagcctggcaggtattgatgatgatagcgttgataaacgtagccagatttgccgtaaaattgttgaagcatgtgaagattggggcatttttcaggttgttgatcatggcattgatatcgatctgattagcgaaatgacccgtctggcacgtcagttttttgcactgcctgcagaagaaaaactgcgttttgatatgaccggtggtaaaaaaggtggttttattgttagcagccatctgcagggtgaagcagttcaggattggcgtgaaattgttacctatttcagctatccgattcaggcacgtgattatagccgttggcctgataaaccggaaggttggcgtagcattaccgaaatgtatagtgatgaactgatggcactggcatgtaaactgctggaagttctgagcgaagcaatgggtctggaaaaagagggtctgaccaaagcatgtgttgatatggatcagaaagtgatcgtgaactactatccgaaatgtccgcagccgaatctgaccctgggtctgaaacgtcataccgatccgggtacaattaccctgctgctgcaggatcaggttggtggtctgcaggcgacccgtgatggtggcaaaacctggattaccgttcagccggttgaaggtgcatttgttgttaatctgggtgatcatggtcactatctgagcaatggtcgctttaaaaacgcagatcatcaggcagttgttaatagcaatagcagccgtatgagcattgcaacctttcagaatccggcaccgaatgcaaccgtttatccgctgaaaattcgtgaaggtgaaaaagccgttatggaagaaccgattacctttgccgagatgtataaacgtaaaatgagccgtgatattgaaatggccaccctgaaaaaactggccaaagaaaaagttctgcaggaccaagaagtggaaaaagcaaaactgcagatgaccccgaaaagcgcagatgaaatttttgccctggttggtgcactgatgcatgttatgcagaaacgtagccgtgcaattcatagcagtgatgaaggtgaagatcaagccggtgatgaagatgaggat

SEQ ID NO: 49 - protein, 400 aa, Petroselinum crispum
MAPSTLTALAQEKTLNSKFVRDEDERPKIAYNKFSDEIPVISLAGIDDDSVDKRSQICRKIVEACEDWGIFQVVDHGIDIDLISEMTRLARQFFALPAEEKLRFDMTGGKKGGFIVSSHLQGEAVQDWREIVTYFSYPIQARDYSRWPDKPEGWRSITEMYSDELMALACKLLEVLSEAMGLEKEGLTKACVDMDQKVIVNYYPKCPQPNLTLGLKRHTDPGTITLLLQDQVGGLQATRDGGKTWITVQPVEGAFVVNLGDHGHYLSNGRFKNADHQAVVNSNSSRMSIATFQNPAPNATVYPLKIREGEKAVMEEPITFAEMYKRKMSRDIEMATLKKLAKEKVLQDQEVEKAKLQMTPKSADEIFALVGALMHVMQKRSRAIHSSDEGEDQAGDEDED

SEQ ID NO: 50 - DNA, 1074 bp, Anthurium andraeanum
  source 1..1074: mol_type = genomic DNA
atgatgcataaaggcaccgtttgtgttaccggtgcagcaggttttgttggtagctggctgattatgcgtctgctggaacagggttatagcgttaaagcaaccgttcgtgatccgagcaatatgaaaaaagttaaacatctgctggatctgcctggtgcagcaaatcgtctgaccctgtggaaagcagatctggttgatgaaggtagctttgatgaaccgattcagggttgtaccggtgtttttcatgttgcaaccccgatggattttgaaagcaaagatccggaaagcgaaatgattaaaccgaccattgaaggtatgctgaatgttctgcgtagctgtgcccgtgcaagcagcaccgttcgtcgtgttgtttttaccagcagcgcaggtacagttagcattcatgaaggtcgtcgtcatctgtatgatgaaaccagttggagtgatgttgatttttgccgtgccaaaaaaatgaccggctggatgtattttgttagcaaaaccctggcagaaaaagcagcatgggattttgcagagaaaaataacatcgacttcatcagcattattccgaccctggttaatggtccgtttgttatgccgaccatgcctccgagcatgctgagcgcactggcactgattacccgtaatgaaccgcattatagcattctgaatccggtgcagtttgttcatctggatgatctgtgtaacgcccacatttttctgtttgaatgtccggatgcaaaaggtcgttatatttgtagcagccatgatgttaccattgcaggtctggcacagattctgcgtcagcgttatccggaatttgatgttccgaccgaatttggtgaaatggaagtgtttgatatcatcagctatagcagcaaaaaactgacggatctgggtttcgaattcaaatatagcctggaagatatgttcgatggtgcaattcagagctgtcgtgaaaaaggtctgctgcctccggcaaccaaagaaccgagctatgcaaccgaacagctgattgcaaccggtcaggataatggtcatcctcctcctgcactgcctccgaaacgtcgtcgt

SEQ ID NO: 51 - protein, 358 aa, Anthurium andraeanum
MMHKGTVCVTGAAGFVGSWLIMRLLEQGYSVKATVRDPSNMKKVKHLLDLPGAANRLTLWKADLVDEGSFDEPIQGCTGVFHVATPMDFESKDPESEMIKPTIEGMLNVLRSCARASSTVRRVVFTSSAGTVSIHEGRRHLYDETSWSDVDFCRAKKMTGWMYFVSKTLAEKAAWDFAEKNNIDFISIIPTLVNGPFVMPTMPPSMLSALALITRNEPHYSILNPVQFVHLDDLCNAHIFLFECPDAKGRYICSSHDVTIAGLAQILRQRYPEFDVPTEFGEMEVFDIISYSSKKLTDLGFEFKYSLEDMFDGAIQSCREKGLLPPATKEPSYATEQLIATGQDNGHPPPALPPKRRR

SEQ ID NO: 52 - DNA, 1074 bp, Camellia sinensis
  source 1..1074: mol_type = genomic DNA
atgaaagatagcgttgcaagcgcaaccgcaagcgcaccgggtacagtttgtgttaccggtgcagcaggttttattggtagctggctggttatgcgtctgctggaacgtggttatattgttcgtgcaaccgttcgtgatccggcaaatctgaaaaaagttaaacatctgctggatctgccgaaagcagataccaatctgaccctgtggaaagccgatctgaatgaagagggtagctttgatgaagcaattgaaggttgtagcggtgtttttcatgttgcaaccccgatggattttgaaagcaaagatccggaaaacgaagtgattaaaccgaccattaacggtgtgctgagcattattcgtagctgtaccaaagcaaaaaccgttaaacgtctggtttttaccagcagcgcaggtacagttaatgttcaagaacatcagcagccggtgtttgatgaaaacaattggagcgatctgcacttcatcaacaaaaaaaaaatgaccggctggatgtattttgtgagcaaaaccctggcagaaaaagcagcatgggaagcagcaaaagaaaacaacattgatttcatcagcattatcccgaccctggttggtggtccgtttattatgccgacctttccgcctagcctgattaccgcactgagcccgattacccgtaatgaaggtcattattccattatcaaacagggccagtttgtgcatctggatgatctgtgtgaaagccacatttttctgtatgaacgtccgcaggcagaaggtcgttatatttgtagcagccatgatgcaaccattcatgatctggccaaactgatgcgtgaaaaatggcctgaatataatgttccgaccgaattcaaaggcatcgataaagatctgccggttgttagcttttccagcaaaaaactgattggcatgggcttcgagttcaaatatagcctggaagatatgtttcgtggtgccattgatacctgtcgtgaaaaaggtctgctgccgcatagctttgcagaaaatccggttaatggcaacaaagtgcctcctcctgcactgcctccgaaacgtcgtcgt

SEQ ID NO: 53 - protein, 358 aa, Camellia sinensis
MKDSVASATASAPGTVCVTGAAGFIGSWLVMRLLERGYIVRATVRDPANLKKVKHLLDLPKADTNLTLWKADLNEEGSFDEAIEGCSGVFHVATPMDFESKDPENEVIKPTINGVLSIIRSCTKAKTVKRLVFTSSAGTVNVQEHQQPVFDENNWSDLHFINKKKMTGWMYFVSKTLAEKAAWEAAKENNIDFISIIPTLVGGPFIMPTFPPSLITALSPITRNEGHYSIIKQGQFVHLDDLCESHIFLYERPQAEGRYICSSHDATIHDLAKLMREKWPEYNVPTEFKGIDKDLPVVSFSSKKLIGMGFEFKYSLEDMFRGAIDTCREKGLLPHSFAENPVNGNKVPPPALPPKRRR

SEQ ID NO: 54 - DNA, 1056 bp, Fragaria x ananassa
  source 1..1056: mol_type = genomic DNA
atgggtctgggtgcagaaagcggtagcgtttgtgttaccggtgcaagcggttttgttggtagctggctggttatgcgtctgctggaacatggttataccgttcgtgcaaccgtgcgtgatccggcaaatctgaaaaaagttcgtcatctgctggaactgccgcaggcagcaacccgtctgaccctgtggaaagcagatctggatgttgaaggtagctttgatgaagccattaaaggttgtaccggtgtttttcatgttgcaaccccgatggattttgaaagcgaagatccggaaaacgaagttattaaaccgaccattaacggcatgctggatattatgaaagcatgcctgaaagcaaaaaccgttcgtcgtctggtttttaccagcagtgccggtgcagttgcaattgaagaacatccgaaagaagtgtacagcgaaaataactggtcagatgttgtgttttgccgcaaagttaaaatgaccggctggatgtattttgtgagcaaaaccctggcagaacaggcagcatggaaatttgcaaaagaaaacaacatcgacttcatcaccattattccgaccctggttattggtccgtttctggcaccgagcatgcctccgagcctgattagcggtctgagtccgctgaccggtaatgaagcacattatggtattatcaaacagtgccagtatgtgcatctggatgatctgtgtcagagccatatttttctgtatgaacatgcaaaagccgagggtcgttatatttgtagcagccatgatgcaaccattcacgatattgcaaaactgctgaacgagaaatacccgaaatacaacgttccgaaaaaattcaaaggcatcgaagaaaacctgaccaacattcactttagcagcaaaaaactgaaagagatgggcttcgaatttaaacacagcctggaagatatgtttacaggtgccgttgatgcatgtcgtgaaaaaggtctgctgccgctgccgcaagaagaagaaaccgaaaaacgtcgtgcaggtcctcctcctgcactgcctccgaaacgtcgtcgt

SEQ ID NO: 55 - protein, 352 aa, Fragaria x ananassa
MGLGAESGSVCVTGASGFVGSWLVMRLLEHGYTVRATVRDPANLKKVRHLLELPQAATRLTLWKADLDVEGSFDEAIKGCTGVFHVATPMDFESEDPENEVIKPTINGMLDIMKACLKAKTVRRLVFTSSAGAVAIEEHPKEVYSENNWSDVVFCRKVKMTGWMYFVSKTLAEQAAWKFAKENNIDFITIIPTLVIGPFLAPSMPPSLISGLSPLTGNEAHYGIIKQCQYVHLDDLCQSHIFLYEHAKAEGRYICSSHDATIHDIAKLLNEKYPKYNVPKKFKGIEENLTNIHFSSKKLKEMGFEFKHSLEDMFTGAVDACREKGLLPLPQEEETEKRRAGPPPALPPKRRR

SEQ ID NO: 56 - DNA, 1062 bp, Camellia sinensis
  source 1..1062: mol_type = genomic DNA
atggcaatggccatggcaaccaccaccacaaccaccaaaccgatgattggtgcaaaagcagcatgtgttgttggtggcaccggttttgttgcagcaaccctggttaaaatgctgctggaacgtggttatagcgttaataccaccgttcgtgatccggacaacaaaaaaaacattagccatctggttgcactggaaggtatgggtaatctgaaaatctttcgtgcagatctgaccgatgaacagagctttgatgcaccgattgcaggttgtgatctggtttttgatgttgccacaccggttaattttgcaagcgaagatccggaaaacgacatgattaaactggcaattcagggtgttctgaatgtgctgaaagcatgtgccaaagcaggcaccgttaaacgtgttattctgaccagcagcgcagcaagcgttaccattaatcagctggatggtacaggtctggttatggatgaaagccattggagtgatgttgaatttctgacctcagttaaaccgcctacctggggtcatccggttagcaaaaccctggcagaaaaagcagcctggaaatttgcagaagaaaataacctgaatctgattaccgttgttccgaccctgaccgcaggtccgagcctgaccagcgaagttccgaatagcattgaactggccatgagcctgattacgggtaatgaattcctgattgatggtctgaaaggtatgcgtattctgtcaggtagcattagcattacccatgttgaagatgtttgtggtgcccatatttttgtggccgaaaaagaaagcgcaagcggtcgttatatttgttgtggtgttaatagcagcgtgccggaactggcacgttttctgaataaacgttatccgcagtataatgtgccgaccgattttggtgatctgccgagcaaagcaaaactgattattagcagcgagaaactgatcaaagaaggcttcagcttcaaatatggcatcgaagaaatttttgcacacagcgttgcatatctgaaaaccaaaggtctgctgcagaacggtgttaaagaaagcctggtt

SEQ ID NO: 57 - protein, 354 aa, Camellia sinensis
MAMAMATTTTTTKPMIGAKAACVVGGTGFVAATLVKMLLERGYSVNTTVRDPDNKKNISHLVALEGMGNLKIFRADLTDEQSFDAPIAGCDLVFDVATPVNFASEDPENDMIKLAIQGVLNVLKACAKAGTVKRVILTSSAASVTINQLDGTGLVMDESHWSDVEFLTSVKPPTWGHPVSKTLAEKAAWKFAEENNLNLITVVPTLTAGPSLTSEVPNSIELAMSLITGNEFLIDGLKGMRILSGSISITHVEDVCGAHIFVAEKESASGRYICCGVNSSVPELARFLNKRYPQYNVPTDFGDLPSKAKLIISSEKLIKEGFSFKYGIEEIFAHSVAYLKTKGLLQNGVKESLV

SEQ ID NO: 58 - DNA, 1167 bp, Desmodium uncinatum
  source 1..1167: mol_type = genomic DNA
atgaccgttagcggtgcaattccgagcatgaccaaaaatcgtaccctggttgttggtggcaccggttttattggtcagtttattaccaaagcaagcctgggttttggttatccgacctttctgctggttcgtccgggtccggttagcccgagcaaagcagttattatcaaaacctttcaggataaaggtgccaaagtgatttatggcgtgatcaacgataaagaatgcatggaaaaaattctgaaagagtacgagatcgacgttgttattagcctggtgggtggtgcacgtctgctggatcagctgaccctgctggaagcaattaaaagcgttaaaaccatcaaacgttttctgccgagcgaatttggccatgatgttgatcgtaccgatccggttgaaccgggtctgaccatgtataaagaaaaacgtctggtgcgtcgtgccgttgaagaatatggtattccgtttaccaatatctgctgcaatagcattgcaagctggccgtattatgataattgtcatccgagccaggttccgcctccgatggatcagtttcagatttatggtgatggtaacaccaaagcctatttcattgatggcaacgatatcggcaaatttaccatgaaaaccatcgatgatattcgcaccctgaacaaaaatgttcattttcgtccgagcagcaactgctacagcattaatgaactggcaagcctgtgggagaaaaaaatcggtcgtacactgcctcgttttaccgttaccgcagataaactgctggcacatgcagcagaaaacattattccggaaagcattgttagcagctttacccacgatatctttattaacggttgccaggtgaactttagcatcgatgaacatagtgatgtggaaatcgatacactgtatccggatgaaaaatttcgtagcctggatgattgctatgaagattttgttccgatggtgcacgataaaattcatgcaggtaaaagcggtgaaatcaaaatcaaagatggtaaaccgctggttcagaccggcaccattgaagaaattaacaaagacattaaaaccctggtggaaacccagccgaatgaagagatcaaaaaagatatgaaagcactggttgaagccgttccgattagcgcaatgggtggtgttaaagaaagcctggtt

SEQ ID NO: 59 - protein, 389 aa, Desmodium uncinatum
MTVSGAIPSMTKNRTLVVGGTGFIGQFITKASLGFGYPTFLLVRPGPVSPSKAVIIKTFQDKGAKVIYGVINDKECMEKILKEYEIDVVISLVGGARLLDQLTLLEAIKSVKTIKRFLPSEFGHDVDRTDPVEPGLTMYKEKRLVRRAVEEYGIPFTNICCNSIASWPYYDNCHPSQVPPPMDQFQIYGDGNTKAYFIDGNDIGKFTMKTIDDIRTLNKNVHFRPSSNCYSINELASLWEKKIGRTLPRFTVTADKLLAHAAENIIPESIVSSFTHDIFINGCQVNFSIDEHSDVEIDTLYPDEKFRSLDDCYEDFVPMVHDKIHAGKSGEIKIKDGKPLVQTGTIEEINKDIKTLVETQPNEEIKKDMKALVEAVPISAMGGVKESLV

SEQ ID NO: 60 - DNA, 1296 bp, Petunia x hybrida
  source 1..1296: mol_type = genomic DNA
atggtgaatgcagtagttacaactccttcaagagttgaaagcttggctaaaagtggaatccaggccatccctaaggagtatgtgaggccacaagaagagttgaatggaatcggaaacatcttcgaggaagagaagaaagatgaagggcctcaagtaccaacaattgatttgaaagaaattgactccgaggacaaggagattcgcgagaaatgccaccaggagttgaagaaagcagccatggaatggggtgtcatgcaccttgtgaatcatggcatatccgatgagctaatcaatcgtgtcaaggttgctggagagaccttctttgatcaacctgttgaagaaaaggagaagtatgctaatgaccaagccaatggcaatgtccaaggctacggcagcaagctagcaaatagtgcttgtggtcagcttgagtgggaggattatttcttccattgtgctttccctgaagacaagcgcgacttgtccatctggcctaaaaatcctactgactacactccagcaacaagtgaatatgccaagcagatcagggccctagcaacaaagattttgacagtgctttctattgggctggggctggaagaaggaagactagagaaggaagttggaggcatggaggatctgctgcttcaaatgaagattaactactatcccaagtgcccccaaccagaactagcacttggcgtcgaagctcatacagatgtcagcgcactgactttcatcctccacaatatggtgcccggcttgcaactcttctatgaaggccagtgggtaactgctaagtgtgtgcctaattctatcatcatgcacataggggacaccattgaaatcctaagcaatggaaagtacaagagcatccttcatagaggggttgtgaataaagagaaagtaaggatctcatgggccattttctgcgagccacctaaggagaagatcatccttaagcccctacctgagactgtcactgaggctgagccacctcgattcccacctcgcacctttgcacagcatatggcacacaagctcttcaggaaggatgacaaggatgccgctgttgaacacaaagtcttcaaagaggatgaactggatactgctgctgaacataaggtcctcaagaaggataatcaggatgctgttgctgagaataaagacatcaaggaggatgaacagtgtggccctgctgagcacaaagatatcaaggaggatggacagggtgccgctgctgagaacaaagtcttcaaggagaataatcaggatgttgctgctgaagaatctaaatag

SEQ ID NO: 61 - protein, 431 aa, Petunia x hybrida
MVNAVVTTPSRVESLAKSGIQAIPKEYVRPQEELNGIGNIFEEEKKDEGPQVPTIDLKEIDSEDKEIREKCHQELKKAAMEWGVMHLVNHGISDELINRVKVAGETFFDQPVEEKEKYANDQANGNVQGYGSKLANSACGQLEWEDYFFHCAFPEDKRDLSIWPKNPTDYTPATSEYAKQIRALATKILTVLSIGLGLEEGRLEKEVGGMEDLLLQMKINYYPKCPQPELALGVEAHTDVSALTFILHNMVPGLQLFYEGQWVTAKCVPNSIIMHIGDTIEILSNGKYKSILHRGVVNKEKVRISWAIFCEPPKEKIILKPLPETVTEAEPPRFPPRTFAQHMAHKLFRKDDKDAAVEHKVFKEDELDTAAEHKVLKKDNQDAVAENKDIKEDEQCGPAEHKDIKEDGQGAAAENKVFKENNQDVAAEESK

SEQ ID NO: 62 - DNA, 1383 bp, Arabidopsis thaliana
  source 1..1383: mol_type = genomic DNA
atgaccaaaccctccgacccaaccagagactcccacgtggcagttctcgcttttcctttcggcactcatgcagctcctctcctcaccgtcacgcgccgcctcgcctccgcctctccttccaccgtcttctctttcttcaacaccgcacaatccaactcttcgttattttcctccggtgacgaagcagatcgtccggcgaacatcagagtatacgatattgccgacggtgttccggagggatacgtgtttagcgggagaccacaggaggcgatcgagctgtttcttcaagctgcgccggagaatttccggagagaaatcgcgaaggcggagacggaggttggtacggaagtgaaatgtttgatgactgatgcgttcttctggttcgcggctgatatggcgacggagataaatgcgtcgtggattgcgttttggaccgccggagcaaactcactctctgctcatctctacacagatctcatcagagaaaccatcggtgtcaaagaagtaggtgagcgtatggaggagacaataggggttatctcaggaatggagaagatcagagtcaaagatacaccagaaggagttgtgtttgggaatttagactctgttttctcaaagatgcttcatcaaatgggtcttgctttgcctcgtgccactgctgttttcatcaattcttttgaagatttggatcctacattgacgaataacctcagatcgagatttaaacgatatctgaacatcggtcctctcgggttattatcttctacattgcaacaactagtgcaagatcctcacggttgtttggcttggatggagaagagatcttctggttctgtggcgtacattagctttggtacggtcatgacaccgcctcctggagagcttgcggcgatagcagaagggttggaatcgagtaaagtgccgtttgtttggtcgcttaaggagaagagcttggttcagttaccaaaagggtttttggataggacaagagagcaagggatagtggttccatgggcaccgcaagtggaactgctgaaacacgaagcaacgggtgtgtttgtgacgcattgtggatggaactcggtgttggagagtgtatcgggtggtgtaccgatgatttgcaggccattttttggggatcagagattgaacggaagagcggtggaggttgtgtgggagattggaatgacgattatcaatggagtcttcacgaaagatgggtttgagaagtgtttggataaagttttagttcaagatgatggtaagaagatgaaatgtaatgctaagaaacttaaagaactagcttacgaagctgtctcttctaaaggaaggtcctctgagaatttcagaggattgttggatgcagttgtaaacattatttga

SEQ ID NO: 63 - protein, 460 aa, Arabidopsis thaliana
MTKPSDPTRDSHVAVLAFPFGTHAAPLLTVTRRLASASPSTVFSFFNTAQSNSSLFSSGDEADRPANIRVYDIADGVPEGYVFSGRPQEAIELFLQAAPENFRREIAKAETEVGTEVKCLMTDAFFWFAADMATEINASWIAFWTAGANSLSAHLYTDLIRETIGVKEVGERMEETIGVISGMEKIRVKDTPEGVVFGNLDSVFSKMLHQMGLALPRATAVFINSFEDLDPTLTNNLRSRFKRYLNIGPLGLLSSTLQQLVQDPHGCLAWMEKRSSGSVAYISFGTVMTPPPGELAAIAEGLESSKVPFVWSLKEKSLVQLPKGFLDRTREQGIVVPWAPQVELLKHEATGVFVTHCGWNSVLESVSGGVPMICRPFFGDQRLNGRAVEVVWEIGMTIINGVFTKDGFEKCLDKVLVQDDGKKMKCNAKKLKELAYEAVSSKGRSSENFRGLLDAVVNII

SEQ ID NO: 64 - protein, 515 aa, Fragaria x ananassa
MGSAVAVELVFIPAPGVGHIMSTMEMAKLLINRHQSIATTVLLIHPPYSSSVLTNYIQSLLTNPIQRIRFIQLPQDQETASKLDLKAPFTSFYEFINSHRNYVRNVVSDMLSRPGSVRITGLVVDILCTGMIDVANEFSIPSYAFFTSNAAFLGFKLYMDTLCRNQKQEGIIALSKSDGELRIPSFVKPVPMTVYPAVYQTRDGLDFLTVSIQKFREAKAIMVNTFLELETHAIESFSSYTNFPSVYAVGPVLNLNGVAGKDEDKDVIRWLDGQPPSSVVFLCFGSMGSFEEVQLKEIAYALERSGHRFVWSVRRPPSPEQSFKVLPDDYDDPRSILPDGFLERTNGFGKVIGWAPQVSILAHEAVGGFVSHCGWNSVLESICCKVPILAWPMMAEQHLNARMVVEEIKIGLRVETCDGSVRGFVQADGLKKMVKELMEGENGEIVRKRVEGIGEGAKKAMAEGGSSWRTLNELIDELQCVRNSNGGRFPSSEGDSDKSKGESYVPMDNLSLVSI

SEQ ID NO: 65 - protein, 456 aa, Vitis vinifera
MSQTTTNPHVAVLAFPFSTHAAPLLAVVRRLAAAAPHAVFSFFSTSQSNASVFHDSMHTMQCNIKSYDVSDGVPEGYVFAGRPQEDIELFMRAAPEGFRQGMVMAVAETGRPVSCLVADAFIWFAADMAAEMGVAWLPFWTAGPNSLSTHVYTDEIREKIGVSGIQGREDELLNFIPGMYEVRFRDLQEGIVFGNLNSLFSRMLHRMGQVLPKATAVFINSFEELDDSLTNDLKSKLKTYLNIGPFNLITPPPVVPNTTGCLQWLKERKPTSVVYISFGTVTTPPPAELVALAEALEASRVPFIWSLRDKARVHLPEGFLEKTRGYGMVVPWAPQAEVLAHEAVGAFVTHCGWNSLWESVAGGVPLICRPFFGDQRLNGRMVEDVLEIGVRIEGGVFTKSGLMSCFDQILSQEKGKKLRENLRALRETADRAVGPKGSSTENFKTLVDLVSKPKDV

SEQ ID NO: 66 - protein, 454 aa, Forsynthia sp.
MAIHSHIGVLAFPFGTHAAPLLTLVRRLVLDSSSQGITFSFFNTAKSNCAIFSGQEFDNIKAYDVWDGTHEGEAFTGSNILEAMQLFLAATPGNFEKVMKEAEVKNGMKISCLLSDAFLWFTCDLAEERGIPWVSFWTAASCSLSAHMYTDQIWSLMRSTGTAKTEEKTLSFVPGMTSVRFSDLPEEILSDNLESPLTLMIYKMVQKLSKSTAIVVNSFEEIDPVITNDLKSKFQNFLNIGPSILSSPTLSNGDSGQECLLWLEKQRHASVIYISFGTVITPQPREMAGLAEALETGEFPFLWSLRDNAMKLLPDGFLDRTSKFGMIVSWAPQLKVLENPSVGAFITHCGWNSILESISFGVPMICRPFFGDQNLNSKMVEDVWKIGVRLEGGVFTKNGTIEALHSVMLNETGKAIRENINKLKRKAQNAVKFDGTSTKNFRALLELIKSPRGI

SEQ ID NO: 67 - protein, 433 aa, Solanum sp.
  source 1..433: note = Eggplant
MTTSQLHIAFLAFPFGTHATPLLTLVQKISPFLPSSTIFSFFNTSSSNSSIFSKVPNQENIKIYNVWDGVKEGNDTPFGLEAIKLFIQSTLLISKITEEAEEETGVKFSCIFSDAFLWCFLVKLPKKMNAPGVAYWTGGSCSLAVHLYTDLIRSNKETSLKIPGFSSTLSINDIPPEVTAEDLEGPMSSMLYNMALNLHKADAVVLNSFQELDRDPLINKDLQKNLQKVFNIGPLVLQSSRKLDESGCIQWLDKQKEKSVVYLSFGTVTTLPPNEIGSIAEALETKKTPFIWSLRNNGVKNLPKGFLERTKEFGKIVSWAPQLEILAHKSVGVFVTHCGWNSILEGISFGVPMICRPFFGDQKLNSRMVESVWEIGLQIEGGIFTKSGIISALDTFFNEEKGKILRENVEGLKEKALEAVNQMMEVQQKISRF

SEQ ID NO: 68 - protein, 482 aa, unidentified
  source 1..482: note = Gentian
MDQLHVFFFPFLANGHILPTIDMAKLFSSRGVKATLITTHNNSAIFLKAINRSKILGFDISVLTIKFPSAEFGLPEGYETADQARSIDMMDEFFRACILLQEPLEELLKEHRPQALVADLFFYWANDAAAKFGIPRLLFHGSSSFAMIAAESVRRNKPYKNLSSDSDPFVVPDIPDKIILTKSQVPTPDETEENNTHITEMWKNISESENDCYGVIVNSFYELEPDYVDYCKNVLGRRAWHIGPLSLCNNEGEDVAERGKKSDIDAHECLNWLDSKNPDSVVYVCFGSMANFNAAQLHELAMGLEESGQEFIWVVRTCVDEEDESKWFPDGFEKRVQENNKGLIIKGWAPQVLILEHEAVGAFVSHCGWNSTLEGICGGVAMVTWPLFAEQFYNEKLMTDILRTGVSVGSLQWSRVTTSAVVVKRESISKAVRRLMAEEEGVDIRNRAKALKEKAKKAVEGGGSSYSDLSALLVELSSYPHN

SEQ ID NO: 69 - protein, 448 aa, Petunia x hybrida
MTTSQLHIALLAFPFGSHAAPLLTLVQKLSPFLPSDTIFSFFNTSQSNTSIFSEGSKPDNIKVYNVWDGVTETNGNKPVGLEAIKLFIQATPTNFEKVMKEAEEETGVKFSCIFSDAFLWFSYKLAEKINVPWIAFWTAASGSLSVHLYTDFIRSNDETSLNIPGFSSTLKISDMPPEVMAENLDLPMPSMLYNMALNLHKAAAVVLNSFEELDPTINKDLKVKLQKVLNIGPLVLQPTSPKKVLDACDERGCIIWLEKQKEESVVYLSFGTVTTLPPNEIVAVAEALEAKKFPFIWSLKDNGIKNLPTGFLERTGQFGKIVSWAPQLEILNHSAVGVFVTHCGWNSILEGISCGVPMICRPFFGDQKLNSRMVESVWQIGLQIEGGSFTKIGTISALDTFFSEEKGKVLRENVKGLKERALEAVKPDGSSSKNFKDLVELVKCHKLT

SEQ ID NO: 70 - protein, 357 aa, Malus domestica
MVSSDSVNSRVETLAGSGISTIPKEYIRPKDELVNIGDIFEQEKNNEGPQVPTIDLKEIESDNEKVRAKCREKLKKATVDWGVMHLVNHGISDELMDKVRKAGKAFFDLPIEQKEKYANDQASGKIQGYGSKLANNASGQLEWEDYFFHCVYPEDKRDLSIWPQTPADYIEATAEYAKQLRELATKVLKVLSLGLGLDEGRLEKEVGGLEELLLQMKINYYPKCPQPELALGVEAHTDVSALTFILHNMVPGLQLFYEGKWVTAKCVPNSIVMHIGDTLEILSNGKYKSILHRGMVNKEKVRISWAVFCEPPKEKIILKPLPETVSEDEPAMFPPRTFAEHIQHKLFRKSQEALLPK

SEQ ID NO: 71 - protein, 357 aa, Pyrus communis
MVSSDSVNSRVETLAGSGISTIPKEYIRPKDELVNIGDIFEQEKNNEGPQVPTIDLKEIESDNEKVRAKCREELKKAAVDWGVMHLVNHGISDELMDKVRKAGKAFFDLPIEQKEKYANDQASGKIQGYGSKLANNASGQLEWEDYFFHCVYPEDKRDLSIWPQTPADYIEATAEYAKQLRELATKVLKVLSLGLGLDEGRLEKEVGGLEELLLQMKINYYPKCPQPELALGVEAHTDVSALTFILHNMVPGLQLFYEGKWVTAKCVPNSIVMHIGDTLEILSNGKYKSILHRGMVNKEKVRISWAVFCEPPKEKIILKPLPETVSEDEPAMFPPRTFAEHIQHKLFRKSQEALLPK

SEQ ID NO: 72 - protein, 357 aa, Prunus avium
MVSSDSVNSRVETLASSGIATIPKEYIRPKEELINIGDIFEQEKSTDGPQVPTIDLKEIDSENEKVRERCREELNKAAVDWGVMHLVNHGISDELMDRVRKAGKAFFDLPIEQKEKYANDQASGKIQGYGSKLANNASGQLEWEDYFFHLIFPEDKRDLSIWPQTPADYIEATAEYAKELRALATKVLRVLSLGLGLEEGRLEKEVGGLEELLLQMKINYYPVCPQPELALGVEAHTDVSALTFILHNMVPGLQLFYEGKWVTAKCVPNSIVMHIGDTIEILSNGKYKSILHRGMVNKEKVRISWAVFCEPPKEKIILKPLPETVSETEPPIFPPRTFAEHIQHKLFRKSQEALLNK

SEQ ID NO: 73 - protein, 383 aa, Fragaria x ananassa
MVTAASIGSRVESLASSGISTIPKEYVRPEEELVNIGDIFEDEKSTEGPQVPTIDLKEIDSEDIKVREKCREELKKAAIDWGVMHLVNHGISDELMERVKKAGKAFFDLPVEQKEKYANDQASGKIQGYGSKLANNASGQLEWEDYFFHCVYPEDKRDLSIWPQTPSDYIVATSEYAKELRGLTTKILSILSLGLGLEEGRLEKEVGGLEELLLQMKINYYPKCPQPELALGVEAHTDISALTFILHNMVPGLQLFYGGKWVTAKCVPNSVVMHIGDTLEILSNGKYKSILHRGLVNKEKVRISWAVFCEPPKEKIILKPLPETVSEEEPAIFPPRTFFEHIQHKLFRQSQEALVSTKESAALKSTKESALKSTKEAALISTN

SEQ ID NO: 74 - protein, 355 aa, Vitis vinifera
MVTSVAPRVESLSSSGIQSIPKEYIRPQEELTSIGNVFEEEKKDEGPQVPTIDLKDIESEDEVVRERCREELKKAAMEWGVMHLVNHGISDDLINRVKVAGETFFNLPMEEKEKYANDQASGKIAGYGSKLANNASGQLEWEDYFFHLIFPEDKRDMTIWPKTPSDYVPATCEYSVKLRSLATKILSVLSLGLGLEEGRLEKEVGGMEELLLQKKINYYPKCPQPELALGVEAHTDVSALTFILHNMVPGLQLFYEGKWVTAKCVPNSIIMHIGDTIEILSNGKYKSILHRGLVNKEKVRISWAVFCEPPKEKIILKPLPETVSETEPPLFPPRTFSQHIQHKLFRKTQEALLSK

SEQ ID NO: 75 - protein, 371 aa, Ipomoea purpurea
MLSTITATVPSRVERLAGSGIERIPKEYIRPEEERRSIGDIFEEEKIAGGPQVPTVDLKGINSEDLEVREKCREELRKAAVDWGVMHLVNHGIPEELTGRVKAAGEGFFGQPIEEKEKYANDQAAGNVQGYGSKLANNASGQLEWEDYFFHCIFPEDKTDLSIWPKTPSDYIDATREYAKQLRALATKVLAVLSLGLGLEEGRLEKEVGGMEELLLQMKINYYPKCPQPELALGVEAHTDVSALTFILHNMVPGLQLFYGGKWVTAKCVPNSIIMHVGDTVEILSNGKYKSILHRGVVNREKVRVSWAVFCEPPKDKILLQPLPETVSEAEPPRFPPRTFAQHIKHKLFRQSDQEAAHTPKPDNDDDHQSN

SEQ ID NO: 76 - protein, 354 aa, Camellia sinensis
MTTVAAPRVQSLATSGIESIPKEYVRPKEELTGIGNIFEEEKNEEGPQVPTIDLKDIDSEVEEVRERCREALKKAAVDWGVMHLVNHGIADDVRERVKVAGEGFFEQPVEEKEKYANDPDNGNLQGYGSKLANNACGQFEWEDYFFHLAYPEDKCDMSIWPKTPTDYIPATVEYAKQLRALATKTLSILSLGLGLEENKLEKEVGGKEELLLQMKINYYPKCPQPELALGVEAHTDLSAVSFILPSMVPGLQLFYEGKWITAKCVPNSIIMLIGDTVEILSNGKYKSILHRGLVNKEKVRISWAVFCEPPKEKIILKPLPETVSEAEPPLFPPRTFAQHIQHKLFRKSQELGSK

SEQ ID NO: 77 - protein, 357 aa, Citrus sinensis
MVTPTARRVESLARSGIQAIPKEYVRPKEELMGIGNIFEEEEKDEGPQVPTIDLKEIDSEDRVEREKCREELKKAAMDWGVMHLVNHGISDDLTERVKRAGQAFFDQPVEEKEKYANEQASGKIQGYGSKLANNASGQLEWEDYFFHLIYPEDKRDMSIWPKTPSDYTEATSEYARQLRSLATKILAVLSLGLGLEEGRLEKEVGGLEELLLQMKINYYPKCPQPELALGVEAHTDVSALTFILHNMVPGLQLFYKDKWVTAKCVPNSIILHIGDTIEILSNGEYKSILHRGLVNKEKVRISWAVFCEPPKDKIILKPLPETVSEQKPAMFPPRTFQQHIEHKLFRRTQDALLSDEE

SEQ ID NO: 78 - protein, 360 aa, Vaccinium ashei
MVSTMVAAPSRVESLASSGIQSIPKEYVRPKEELTSIGNIFEEEKKHEGPQVPTIDLEDLVSEDKEARERCHEALKKAATEWGVMHLVNHGVPEELMDRVRVAGEGFFNQPVEEKEKYANDHDTGNSGKIQGYGSKLANNASGQLEWEDYFFHTVYPEDKRDMKIWPKNPSDYIPATSEYANHLRALTTKVLSALSVCLGLEEDRLEKEVGGKDELVIQMKINYYPKCPQPELALGVEAHTDVSALTFILHNMVPGLQLFYEGKWITAKCVPNSIIMHIGDTVEILSNGKYKSILHRGLVNKEKVRISWAAFCEPPKEKIILKPLPETVSETEPARYPPRTFSQHIEHKLFRKTQALNGA

SEQ ID NO: 79 - protein, 361 aa, Populus trichocarpa
MMVTSSFVVPRVESLASSGIQSIPKEYIRPQEELSSIRDVFEEEKKVEGPQVPTIDLKEMESEDKVVREKCREELVKAATEWGVMHLVNHGIPDDLIDRVKKAGQAFFDLPIEEKEKHANDQASGNVQGYGSKLANNASGQLEWEDYFFHLIFPEDKRDFSIWPKTPSDYTEVTSEYARQLRSLATKILSVLSLGLGLEEGRLEKEVGGLEELLLQMKINYYPKCPQPDLALGVEAHSDVSALTFILHNMVPGLQLLYEGKWITAKCVPNSIIMHIGDTVEILSNGKYKSIIHRGLVNKEKVRISWAVFCEPPKAKIILKPLAEIVTEAEPPLFPPRTFSQHIEHKLFRKTQDSLLPRKAN

SEQ ID NO: 80 - protein, 541 aa, Rhodobacter capsulatus
MLDATIGRKRMTLQSQTAKDCLALDGALTLVQCEAIATHRSRISVTPALRERCARAHARLEHAIAEQRHIYGITTGFGPLANRLIGADQGAELQQNLIYHLATGVGPKLSWAEARALMLARLNSILQGASGASPETIDRIVAVLNAGFAPEVPAQGTVGASGDLTPLAHMVLALQGRGRMIDPSGRVQEAGAVMDRLCGGPLTLAARDGLALVNGTSAMTAIAALTGVEAARAIDAALRHSAVLMEVLSGHAEAWHPAFAELRPHPGQLRATERLAQALDGAGRVCRTLTAARRLTAADLRPEDHPAQDAYSLRVVPQLVGAVWDTLDWHDRVVTCELNSVTDNPIFPEGCAVPALHGGNFMGVHVALASDALNAALVTLAGLVERQIARLTDEKLNKGLPAFLHGGQAGLQSGFMGAQVTATALLAEMRANATPVSVQSLSTNGANQDVVSMGTIAARRARAQLLPLSQIQAILALALAQAMDLLDDPEGQAGWSLTARDLRDRIRAVSPGLRADRPLAGHIEAVAQGLRHPSAAADPPA

SEQ ID NO: 81 - protein, 701 aa, Oryza sp.
  source 1..701: note = Rice
MAGNGPINKEDPLNWGAAAAEMAGSHLDEVKRMVAQFREPLVKIQGATLRVGQVAAVAQAKDAARVAVELDEEARPRVKASSEWILTCIAHGGDIYGVTTGFGGTSHRRTKDGPALQVELLRYLNAGIFGTGSDGHTLPSETVRAAMLVRINTLLQGYSGIRFEILEAITKLLNTGVTPCLPLRGTITASGDLVPLSYIAGLITGRPNAQAISPDGRKVDAAEAFKLAGIEGGFFTLNPKEGLAIVNGTSVGSALAATVMFDANILAVLSEVLSAVFCEVMNGKPEYTDHLTHKLKHHPGSIDAAAIMEHILAGSSFMSHAKKVNEMDPLLKPKQDRYALRTSPQWLGPQIQVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYSSELQYLANPITNHVQSAEQHNQDVNSLGLVSARKTLEAVDILKLMTSTYIVALCQAVDLRHLEENIKSSVKNCVTQVAKKVLTMNPTGDLSSARFSEKNLLTAIDREAVFSYADDPCSANYPLMQKLRAVLVEHALTSGDRRARGLRVLQDHQVRGGAPLCAAPGDRGRPRRRRQRTAPVANRIVESRSFPLYRFVREELGCVFLTGEKLKSPGEECNKVFLGISQGKLIDPMLDCLKEWNGEPLPIN

SEQ ID NO: 82 - protein, 580 aa, Petroselinum sp.
  source 1..580: note = Parsley
FLNAGIFGNGSDNTLPHSATRAAMLVRINTLLQGYSGIRFEILEAITKFLNQNITPCLPLRGTITASGDLVPLSYIAGLLTGRPNSKAVGPTGVILSPEEAFKLAGVEGGFFELQPKEGLALVNGTAVGSGMASMVLFEANILAVLAEVMSAIFAEVMQGKPEFTDHLTHKLKHHPGQIEAAAIMEHILDGSAYVKAAQKLHEMDPLQKPKQDRYALRTSPQWLGPQIEVIRSSTKMIEREINSVNDNPLIDVSRNKAIHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTSEAVEILKLMSTTFLVGLCQAIDLRHLEENLKSTVKNTVSSVAKRVLTMGVNGELHPSRFCEKDLLRVVDREYIFAYIDDPCSATYPLMQKLRQTLVEHALKNGDNERNLSTSIFQKIATFEDELKALLPKEVESARAALESGNPAIPNRIEECRSYPLYKFVRKELGTEYLTGEKVTSPGEEFEKVFIAMSKGEIIDPLLECLESWNGAPLPIC

SEQ ID NO: 83 - protein, 704 aa, Solanum sp.
  source 1..704: note = Tomato
MDLCKKSINDPLNWEMAADSLRGSHLDEVKKMVDEFRKPIVKLGGETLSVAQVASIANVDDKSNGVKVELSESARAGVKASSDWVMDSMSKGTDSYGVTAGFGATSHRRTKNGGALQKELIRFLNAGVFGNGIESFHTLPHSATRAAMLVRINTLLQGYSGIRFEILEAITKLINSNITPCLPLRGTITASGDLVPLSYIAGLLTGRPNSKAVGPNGEKLNAEEAFCVAGISGGFFELQPKEGLALVNGTAVGSAMASIVLFESNIFAVMSEVLSAIFTEVMNGKPEFTDYLTHKLKHHPGQIEAAAIMEHILDGSSYVKVAQKLHEMDPLQKPKQDRYALRTSPQWLGPQIEVIRAATKMIEREINSVNDNPLIDVSRNKALHGGNFQGTPIGVSMDNTRLALASIGKLMFAQFSELVNDYYNNGLPSNLTAGRNPSLDYGFKGAEIAMASYCSELQFLANPVTNHVQSAEQHNQDVNSLGLISARKTAKAVDILKIMSSTYLVALCQAIDLRHLEENLKSVVKNTVSQVAKRTLTMGANGELHPARFSEKELLRVVDREYLFAYADDPCSSNYPLMQKLRQVLVDQAMKNGESEKNVNSSIFQKIGAFEDELIAVLPKEVESVRAVFESGNPLIRNRITECRSYPLYRLVREELGTELLTGEKVRSPGEEIDKVFTAICNGQIIDPLLECLKSWNGAPLPIC

SEQ ID NO: 84 - protein, 725 aa, Arabidopsis sp.
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDSYGVTTGFGATSHRRTKNGVALQKELIRFLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAVMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIRAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALVNGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

(19) United States
(12) Patent Application Publication
(10) Pub. No.: US 2025/0051819 A1
(43) Pub. Date: Feb. 13, 2025

(54) MICROBIAL POLYCULTURES AND METHODS OF USE THEREOF

(71) Applicant: Rensselaer Polytechnic Institute, Troy, NY (US)

(72) Inventors: Mattheos KOFFAS, Niskayuna, NY (US); John Andrew JONES, Liberty Township, OH (US)

(73) Assignee: Rensselaer Polytechnic Institute, Troy, NY (US)

(21) Appl. No.: 18/617,783

(22) Filed: Mar. 27, 2024

Related U.S. Application Data
(63) Continuation of application No. 17/197,468, filed on Mar. 10, 2021, now Pat. No. 11,965,197.
Application No. 17/197,468 is a continuation of application No. 15/341,911, filed on Nov. 2, 2016, now Pat. No. 10,954,543.
(60) Provisional application No. 62/249,476, filed on Nov. 2, 2015.

(51) Int. Cl. C12P 19/58; C12N 9/00; C12N 9/02; C12N 9/04; C12N 9/10; C12N 9/88; C12N 9/90; C12N 15/52; C12P 7/42; C12P 7/52; C12P 17/06

(52) U.S. Cl. CPC C12P 19/58; C12N 9/0006; C12N 9/0071; C12N 9/0093; C12N 9/1037; C12N 9/88; C12N 9/90; C12N 9/93; C12N 15/52; C12P 7/42; C12P 7/52; C12P 17/06; C12Y 101/01219; C12Y 114/11009; C12Y 117/01003; C12Y 203/01074; C12Y 403/01023; C12Y 505/01006; C12Y 602/01012; C12Y 602/01014

(57) ABSTRACT

Disclosed herein are novel microbial polycultures of two or more cell strains, capable of producinges, flavonoids, and anthocyanidin-3-O-glucosides, and methods of use thereof. Also disclosed is a microbial cell capable of producing phenylpropanoic acids, and methods of use thereof.

### CROSS-REFERENCE TO RELATED APPLICATIONS

This application is a continuation of U.S. Utility patent application Ser. No. 17/197,468, filed Mar. 10, 2021, which is a continuation of U.S. Utility patent application Ser. No. 15/341,911, filed Nov. 2, 2016, now U.S. Pat. No. 10,954,543, which claims the benefit of priority under 35 U.S.C. § 119(e) to U.S. Provisional Patent Application Ser. No. 62/249,476, filed on Nov. 2, 2015, the disclosure of which is incorporated by reference herein in its entirety.

### STATEMENT REGARDING FEDERALLY SPONSORED RESEARCH AND DEVELOPMENT

This invention was made with U.S. Government support under Grant Number DE-AR0000432 awarded by the Department of Energy. The United States Government has certain rights in the invention.

### FIELD OF THE INVENTION

This invention relates to microbial polycultures useful for production of flavanones, flavonoids, and anthocyanidin-3-O-glucosides, and methods of use thereof. This invention also relates to microbial cell useful for production of phenylpropanoic acids, and methods of use thereof.

### INCORPORATION OF SEQUENCE LISTING

The contents of the file named “Sequence_final.xml”, which was created on Oct. 28, 2024 and is 124 KB in size, are hereby incorporated by reference in their entireties.

### BACKGROUND OF THE INVENTION

The microbial production of biofuels, commodity chemicals, and natural products is continually being improved through the use of various pathway optimization tools and techniques. Until recently, these efforts have focused primarily on optimization of single strain monocultures to facilitate conversion of substrate to product. See for example, U.S. Pat. Nos. 7,338,791 and 7,807,422. Although successful, these efforts are continually plagued with the trade-offs associated with choosing a single host strain to simultaneously perform multiple bioconversions, often having different precursor and co-factor requirements.

Nature has overcome these trade-offs through organelle compartmentalization of pathways in higher organisms and through microbial consortia in lower organisms. The presence of microbial communities is ubiquitous in nature. In much the same way that multicellular eukaryotic organisms have evolved to contain specialized organelles that work together to seamlessly perform their specialized tasks; communities of unicellular organisms have developed similar divisions within their populations, such that the consortia of microbes is more than simply a sum of its individual parts. These complex consortia allow for a cellular specialization enabling the community to withstand larger environmental perturbations and perform more complex tasks than any of its individual constituents. Employing this division of labor approach allows for burden to be distributed across the population permitting for increased efficiency and more complex behavior than is possible in monoculture.

Humans have taken advantage of co-culture approaches for wastewater treatment and fermented food products for decades. However, only recently have scientists begun to investigate the true potential of co-culture techniques in metabolic engineering and synthetic biology applications. Several groups have reported elegant applications utilizing co-cultures for the production of pharmaceutical precursors, commodity chemicals, and potential biofuels. In one such example, a S. cerevisiae-E. coli co-culture was engineered to take advantage of rapid taxadiene production from E. coli and the ability of S. cerevisiae to actively express cytochrome P450s to catalyze taxadiene functionalization into oxygenated taxanes. These steps have proven to be inefficient or impossible to accomplish in either a S. cerevisiae or E. coli monoculture. Albeit impressive, previous studies have lacked the rigorous optimization necessary to fully realize the complete production potential of these co-culture systems.

Although the study and application of natural microbial consortia have been a topic of interest in the scientific literature for decades, the development of synthetic consortium, and specifically consortia for metabolic engineering applications, has gained significant traction in the past three years. Several excellent examples of employing microbial communities for metabolic engineering have resulted in significant improvements over monoculture efforts. These gains were realized through utilizing the key advantages of microbial consortia, including: (1) selection of the most efficient organism for the bioconversion (i.e. mixing bacterial and fungal hosts in a single consortium); (2) using traditional metabolic engineering principles (Push, Pull, Block) to optimize each module for its specific co-factor and precursor needs; and (3) taking advantage of consortia modularity such that individual strains can be genetically optimized in monoculture then applied in mixed culture without the need to re-perform the genetic optimization.

However, there is still a need for efficient production of various useful compounds, such as flavanones, flavonoids, and anthocyanidin-3-O-glucosides. Accordingly, there is a need for development of polycultures, two or more strains in co-culture, for production of such useful compounds. Additionally, there is a need for cultures that can produce phenylpropanoic acids, which are also useful for various applications.

### SUMMARY OF THE INVENTION

The present invention relates to microbial polycultures useful production of various useful compounds. Accordingly, in one embodiment, the present invention relates to a method of producing a product compound in a microbial polyculture;
wherein, optionally, the microbial polyculture includes a TAL module cell including an exogenous gene encoding for a tyrosine ammonia lyase (TAL); wherein, optionally, the microbial polyculture includes a C5 module cell including an exogenous gene encoding for a 4-coumaroyl-CoA ligase (4CL), an exogenous gene encoding for a chalcone synthase (CHS), an exogenous gene encoding for a chalcone isomerase (CHI), and wherein, optionally, the C5 module cell further includes an exogenous gene encoding for malonyl-CoA synthetase (MatB) and an exogenous gene encoding for putative dicarboxylate carrier protein (MatC); wherein, optionally, the microbial polyculture further includes a p168 module cell including an exogenous gene encoding for a flavanone 3β-hydroxylase (F3H), an exogenous gene encoding for a dihydroflavonol 4-reductase (DFR), and an exogenous gene encoding for a leucoanthocyanidin reductase (LAR); and wherein, optionally, the microbial polyculture further includes an Antho module cell including an exogenous gene encoding for an anthocyanidin synthase (ANS) and an exogenous gene encoding for a 3-glucosyl transferase (3GT); the method including:
providing a substrate to the microbial polyculture;
culturing the microbial polyculture under conditions permitting synthesis of the product compound by the microbial polyculture; and
isolating the product compound synthesized by the microbial polyculture; with a proviso (i.e., a condition) that:
the microbial polyculture includes the TAL module cell and the C5 module cell, the substrate is glucose, glycerol, or a combination thereof, and the product compound is a flavanone; or
the microbial polyculture includes the C5 module cell and the p168 module cell, the substrate is phenylpropanoic acid, and the product compound is a flavonoid; wherein, when the C5 module cell includes an exogenous gene encoding for malonyl-CoA synthetase (MatB) and an exogenous gene encoding for putative dicarboxylate carrier protein (MatC), the substrate is a phenylpropanoic acid, malonate, or a combination thereof and the product is a flavonoid; or
the microbial polyculture includes the p168 module cell and the Antho module cell, the substrate is a flavanone, and the product compound is an anthocyanidin-3-O-glucoside; or
the microbial polyculture includes the TAL module cell, the C5 module cell, and the p168 module cell, the substrate is glucose, glycerol, or a combination thereof, and the product compound is a flavonoid; or
the microbial polyculture includes the C5 module cell, the p168 module cell, and the Antho module cell, the substrate is a phenylpropanoic acid, and the product compound is an anthocyanidin-3-O-glucoside; wherein, when the C5 module cell includes an exogenous gene encoding for malonyl-CoA synthetase (MatB) and an exogenous gene encoding for putative dicarboxylate carrier protein (MatC), the substrate is phenylpropanoic acid, malonate, or a combination thereof and the product is an anthocyanidin-3-O-glucoside; or
the microbial polyculture includes the TAL module cell, the C5 module cell, the p168 module cell, and the Antho module cell, the substrate is glucose, glycerol, or a combination thereof, and the product compound is an anthocyanidin-3-O-glucoside.

The present invention is also directed to microbial polycultures of the above described methods.

Furthermore, the present invention is also directed to a method of producing a phenylpropanoic acid in a TAL module cell, wherein the TAL module cell is a microbial cell including an exogenous gene encoding for a tyrosine ammonia lyase (TAL); the method including: providing a substrate to the TAL module cell, wherein the substrate includes glucose, glycerol, or a combination thereof; culturing the TAL module cell under conditions permitting synthesis of the phenylpropanoic acid by the TAL module cell; and isolating the phenylpropanoic acid synthesized by the TAL module cell. In one embodiment, the method further includes creating the TAL module cell by introducing an exogenous gene encoding for a tyrosine ammonia lyase (TAL) into a host cell for the TAL module cell.

The present invention is also directed to a TAL module cell, wherein the TAL module cell is a microbial cell including an exogenous gene encoding for a tyrosine ammonia lyase (TAL).

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates flavonoid pathway highlighting upstream (left) malonyl-CoA dependent and downstream (right) NADPH dependent co-culture modules.

FIGS. 2A-2C show upstream strain optimization and co-culture compatibility determination. (A) Screening of twelve potential upstream homolog combinations resulted in several high-titer pathways. (B) Application of ePathOptimize technique for transcriptional optimization resulted in high sensitivity to changes in the transcriptional landscape. (C) Lead strains from the individual strain optimization studies were grown in co-culture to determine strain compatibility prior to additional fermentation optimization. All data was obtained in AMM −2% glucose, 30° C. induction temperature. Error bars represent ±1 standard deviation from duplicate experiments.

FIGS. 3A-3D show sensitivity to induction point, inoculation ratio, and induction temperature for the co-culture system. (A) Variations in induction point and inoculation ratio demonstrate orthogonal response in product titer. Data obtained in glucose only media at an induction temperature of 30° C. (B-D) Variations in the induction temperature show significant shifts to the magnitude and profile of the production landscape. Data obtained in glycerol only media. (B) 10° C. induction temperature. (C) 20° C. induction temperature. (D) 30° C. induction temperature. Data labels represent the highest titer reported in each window. Error bars represent ±1 standard deviation from duplicate or greater (n>2) experiments.

FIGS. 4A-4E show effect of carbon source composition on product titer and the shape of the production landscape. (A-E) Increasing the proportion of glycerol in the production media results in higher titers, later induction point optimums, and optimum inoculation ratios with higher proportion of the downstream strain. (A) Glucose Only. (B) 1:1 Glucose: Glycerol. (C) 1:3 Glucose: Glycerol. (D) 1:9 Glucose: Glycerol. (E) Glycerol Only. Data labels represent the highest titer reported in each window. Error bars represent ±1 standard deviation from duplicate or greater (n>2) experiments.

FIG. 5 shows a polyculture schematic representing the realized 4-strain polyculture. Inclusion of fifth strain shows potential for extension through addition of sequential modules.

FIG. 6 shows screening of potential phenylpropanoic acid production modules. Initial screening was completed under optimal conditions for C5 and p168 co-culture (AMM-2% glycerol, 5-hour induction point, 30° C. fermentation temperature post induction with 1 mM IPTG). Constitutive expression modules (Q/R 11-14) were not induced with IPTG. Titers reported are after 2 days of cultivation in 48-well plates.

FIGS. 7A-7B show analysis of top phenylpropanoic acid production modules. (A) Glucose carbon source, 37° C., Induction 3 hr (R2 and R4 only) (B) Glycerol carbon source, 37° C., Induction 8 hr (R2 and R4 only).

FIG. 8 shows production landscape of three-strain polyculture for the de novo production of (+)-Afzelechin. All data obtained in AMM-Glucose media at a production temperature of 30° C. Error bars represent one standard deviation of at least biological duplicate.

FIG. 9 shows production of anthocyanidin-3-glucosides from glucose using a four-strain polyculture. All data was obtained using a 5-hour induction point and 30° C. induction temperature. Error bars represent ±1 standard deviation from the mean of biological quadruplicates.

### DETAILED DESCRIPTION OF THE INVENTION

We developed and optimized polycultures for the efficient production of various compound types, including flavonoids. Flavonoids are high-value molecules with promising potential for pharmaceutical applications resulting from interesting bioactivity. In the case of flavan-3-ols, a subclass of flavonoid molecules, high-titer production has been achieved from both the malonyl-CoA requiring upstream module (phenylpropanoic acids to flavanones) and the NADPH requiring downstream module (flavanones to flavan-3-ols). However, when the complete pathway is expressed in monoculture, reported titers for flavan-3-ols from phenylpropanoic acids are greater than three orders of magnitude lower than the independent modules. This observation motivated the choice to attempt co-culture production of flavan-3-ols in E. coli.

To accomplish this task, careful experimental optimization of carbon source, induction temperature, induction point, inoculation ratio, and strain choice was used to map the production landscape. The experimental optimization was coupled with extensive empirical modeling techniques that were applied to predict conditions for optimal production. Searching the solution space surrounding the predicted optimum resulted in a 65% improvement in flavan-3-ol titer to 40.7±0.1 mg/L from p-coumaric acid, representing a 970-fold improvement over previous literature reports.

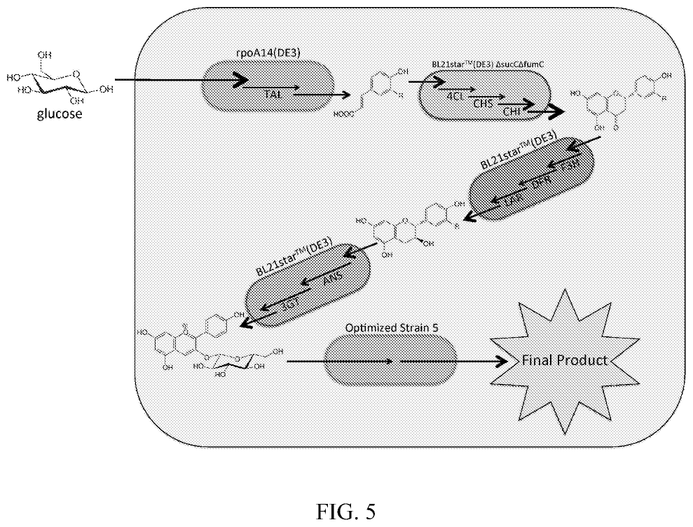

Some of our novel polycultures are also capable of the de novo production of flavan-3-ols and anthocyanidin-3-O-glucosides in microbial hosts, FIG. 5. To accomplish this task, we built off of our previous co-culture demonstration by developing a phenylpropanoic acid production module capable of the highest titer production of p-coumaric and caffeic acid to date. Applying this module, together with the previously developed C5 and p168 modules, enabled production of 26.1 mg/L (+)-afzelechin from glucose. Finally, we further demonstrated the modularity of our system by realizing the production of anthocyanidin-3-glucosides from glucose by introduction of a fourth module for anthocyanin production to the system, resulting in a titer of 12.6±0.4 mg/L pelargonidin-3-O-glucoside de novo. This production was obtained with only minimal fermentation optimization at the polyculture level.

In one embodiment, the invention relates to a method of producing a product compound in a microbial polyculture;
wherein, optionally, the microbial polyculture includes a TAL module cell including an exogenous gene encoding for a tyrosine ammonia lyase (TAL); wherein, optionally, the microbial polyculture includes a C5 module cell including an exogenous gene encoding for a 4-coumaroyl-CoA ligase (4CL), an exogenous gene encoding for a chalcone synthase (CHS), an exogenous gene encoding for a chalcone isomerase (CHI), and wherein, optionally, the C5 module cell further includes an exogenous gene encoding for malonyl-CoA synthetase (MatB) and an exogenous gene encoding for putative dicarboxylate carrier protein (MatC); wherein, optionally, the microbial polyculture further includes a p168 module cell including an exogenous gene encoding for a flavanone 3β-hydroxylase (F3H), an exogenous gene encoding for a dihydroflavonol 4-reductase (DFR), and an exogenous gene encoding for a leucoanthocyanidin reductase (LAR); and wherein, optionally, the microbial polyculture further includes an Antho module cell including an exogenous gene encoding for an anthocyanidin synthase (ANS) and an exogenous gene encoding for a 3-glucosyl transferase (3GT); the method including:
providing a substrate to the microbial polyculture;
culturing the microbial polyculture under conditions permitting synthesis of the product compound by the microbial polyculture; and
isolating the product compound synthesized by the microbial polyculture; with a proviso that:
the microbial polyculture includes the TAL module cell and the C5 module cell, the substrate is glucose, glycerol, or a combination thereof, and the product compound is a flavanone; or
the microbial polyculture includes the C5 module cell and the p168 module cell, the substrate is phenylpropanoic acid, and the product compound is a flavonoid; wherein, when the C5 module cell includes an exogenous gene encoding for malonyl-CoA synthetase (MatB) and an exogenous gene encoding for putative dicarboxylate carrier protein (MatC), the substrate is a phenylpropanoic acid, malonate, or a combination thereof and the product is a flavonoid; or
the microbial polyculture includes the p168 module cell and the Antho module cell, the substrate is a flavanone, and the product compound is an anthocyanidin-3-O-glucoside; or
the microbial polyculture includes the TAL module cell, the C5 module cell, and the p168 module cell, the substrate is glucose, glycerol, or a combination thereof, and the product compound is a flavonoid; or
the microbial polyculture includes the C5 module cell, the p168 module cell, and the Antho module cell, the substrate is a phenylpropanoic acid, and the product compound is an anthocyanidin-3-O-glucoside; wherein, when the C5 module cell includes an exogenous gene encoding for malonyl-CoA synthetase (MatB) and an exogenous gene encoding for putative dicarboxylate carrier protein (MatC), the substrate is phenylpropanoic acid, malonate, or a combination thereof and the product is an anthocyanidin-3-O-glucoside; or
the microbial polyculture includes the TAL module cell, the C5 module cell, the p168 module cell, and the Antho module cell, the substrate is glucose, glycerol, or a combination thereof, and the product compound is an anthocyanidin-3-O-glucoside.

Thus, the microbial polycultures of the invention may be any one of the following polycultures: (1) the TAL module cell and the C5 module cell; (2) the C5 module cell and the p168 module cell; (3) the p168 module cell and the Antho module cell; (4) the TAL module cell, the C5 module cell, and the p168 module cell; (5) the C5 module cell, the p168 module cell, and the Antho module cell; or (6) the TAL module cell, the C5 module cell, the p168 module cell, and the Antho module cell. Use of the singular term “cell” when referring to each module (i.e., TAL module, C5 module, p168 module, and Antho module) is meant to encompass both a single cell of the specified module and a plurality of cells of the specified module.

The TAL module cell includes an exogenous gene encoding for a tyrosine ammonia lyase (TAL). In some embodiments, the exogenous gene encoding for the tyrosine ammonia lyase (TAL) is a gene encoding for Rhodotorula glutinis tyrosine ammonia lyase (RgTAL). In some embodiments, the exogenous gene encoding for the tyrosine ammonia lyase (TAL) is a gene encoding for Rhodotorula glutinis tyrosine ammonia lyase (RgTAL), Rhodobacter capsulatus TAL, Rice TAL, Parsley TAL, Tomato TAL, Arabidopsis TAL, or a combination thereof.

The C5 module cell includes an exogenous gene encoding for a 4-coumaroyl-CoA ligase (4CL), an exogenous gene encoding for a chalcone synthase (CHS), an exogenous gene encoding for a chalcone isomerase (CHI). In addition to these three geness, the C5 module cell may optionally include an exogenous gene encoding for malonyl-CoA synthetase (MatB) and an exogenous gene encoding for putative dicarboxylate carrier protein (MatC).

In some embodiments, the exogenous gene encoding for the 4-coumaroyl-CoA ligase (4CL) is a gene encoding for Arabidopsis thaliana 4-coumaroyl-CoA ligase (At4CL), Petrosehnum crispum 4-coumaroyl-CoA ligase (Pc4CL), Vitis vinifera 4-coumaroyl-CoA ligase (Vv4CL), or a combination thereof. In some embodiments, the exogenous gene encoding for the chalcone synthase (CHS) is a gene encoding for Petunia.times.hybrida chalcone synthase (PhCHS), Citrus maxima chalcone synthase (CmCHS), or a combination thereof. In some embodiments, the exogenous gene encoding for the chalcone isomerase (CHI) is a gene encoding for Medicago sativa chalcone isomerase (MsCHI), Citrus maxima chalcone isomerase (CmCHI), or a combination thereof. In some embodiments, the exogenous gene encoding for the malonyl-CoA synthetase (MatB) is a gene encoding for Rhizobium trifolii malonyl-CoA synthetase (RtMatB). In some embodiments, the exogenous gene encoding for the dicarboxylate carrier protein (MatC) is a gene encoding for Rhizobium trifolii putative dicarboxylate carrier protein (RtMatC).

The p168 module cell includes an exogenous gene encoding for a flavanone 3β-hydroxylase (F3H), an exogenous gene encoding for a dihydroflavonol 4-reductase (DFR), and an exogenous gene encoding for a leucoanthocyanidin reductase (LAR).

In some embodiments, the exogenous gene encoding for the flavanone 3β-hydroxylase (F3H) is a gene encoding for Camellia sinensis flavanone 3β-hydroxylase (CsF3H), Malus domestica flavanone 3β-hydroxylase (MdF3H), Petroselinum crispum flavanone 3β-hydroxylase (PcF3H), or a combination thereof. In some embodiments, the exogenous gene encoding for the dihydroflavonol 4-reductase (DFR) is a gene encoding for Anthrium andraeanum dihydroflavonol 4-reductase (AaDFR), Camellia sinensis dihydroflavonol 4-reductase (CsDFR), Fragaria.times.ananassa dihydroflavonol 4-reductase (FaDFR), or a combination thereof. In some embodiments, the exogenous gene encoding for the leucoanthocyanidin reductase (LAR) is a gene encoding for Camellia sinensis leucoanthocyanidin reductase (CsLAR), Desmodium uncinatum leucoanthocyanidin reductase (DuLAR), or a combination thereof.

The Antho module cell includes an exogenous gene encoding for an anthocyanidin synthase (ANS) and an exogenous gene encoding for a 3-glucosyl transferase (3GT). In some embodiments, the exogenous gene encoding for the anthocyanidin synthase (ANS) is a gene encoding for Petunia.times.hybrida anthocyanidin synthase (PhANS). In some embodiments, the exogenous gene encoding for the anthocyanidin synthase (ANS) is a gene encoding for Petunia.times.hybrida anthocyanidin synthase (PhANS), Malus domestica ANS, Pyrus communis ANS, Prunus avium ANS, Fragaria.times.ananassa ANS, Vitis vinifera ANS, Ipomoea purpurea anthocyanidin synthase (ANS), Camellia sinensis ANS, Citrus sinensis anthocyanidin synthase (ANS), Vaccinium ashei ANS, Populus trichocarpa ANS, or a combination thereof. In some embodiments, the exogenous gene encoding for the 3-glucosyl transferase (3GT) is a gene encoding for Arabidopsis thaliana 3-glucosyl transferase (At3GT). In some embodiments, the exogenous gene encoding for the 3-glucosyl transferase (3GT) is a gene encoding for Arabidopsis thaliana 3-glucosyl transferase (At3GT), Fragaria.times.ananassa 3GT, Vitis vinifera 3GT, Forsynthia 3GT, Eggplant 3GT, Gentian 3GT, Petunia.times.hybrida 3GT, or a combination thereof.

In some embodiments, the exogenous gene encoding for the 4-coumaroyl-CoA ligase (4CL) is a gene encoding for Arabidopsis thaliana 4-coumaroyl-CoA ligase (At4CL); the exogenous gene encoding for the chalcone synthase (CHS) is a gene encoding for Petunia.times.hybrida chalcone synthase (PhCHS); the exogenous gene encoding for the chalcone isomerase (CHI) is a gene encoding for Citrus maxima chalcone isomerase (CmCHI); the exogenous gene encoding for the malonyl-CoA synthetase (MatB) is a gene encoding for Rhizobium trifolii malonyl-CoA synthetase (RtMatB); and the exogenous gene encoding for the dicarboxylate carrier protein (MatC) is a gene encoding for Rhizobium trifolii putative dicarboxylate carrier protein (RtMatC).

In some embodiments, the exogenous gene encoding for the flavanone 3β-hydroxylase (F3H) is a gene encoding for Camellia sinensis flavanone 3β-hydroxylase (CsF3H); the exogenous gene encoding for the dihydroflavonol 4-reductase (DFR) is a gene encoding for Fragaria.times.ananassa dihydroflavonol 4-reductase (FaDFR); and the exogenous gene encoding for the leucoanthocyanidin reductase (LAR) is a gene encoding for Desmodium uncinatum leucoanthocyanidin reductase (DuLAR).

The polypeptides encoded by the exogenous genes in the cells of the polyculture have various known functions. TAL converts tyrosine and phenylalanine to the corresponding phenylpropanoic acids, coumaric acid, and cinnamic acid. 4CL converts phenylpropanoic acids such as coumaric acid, cinnamic acid, ferulic acid, and caffeic acid to their CoA derivatives. CHS performs condensation of phenylpropanoic-CoA derivatives such as coumaroyl-CoA, cinnamoyl-CoA, caffeoyl-CoA, feruloyl-CoA with 3 moles of malonyl CoA and performs Claisen condensation to form chalcones. CHI performs isomerisation of chalcones to flavanones. Math converts the intracellular malonate to malonyl-CoA. MatC transports malonate across the cell membrane. F3H is a dioxygenase that hydroxylates flavanones such as naringenin and eriodictyol to the corresponding dihydroxyflavanones. DFR is a reductase reducing dixydroxyflavanones to the corresponding leucoanthocyanidins. LAR is a reductase that converts leucoanthocyanidins to flavan-3-ols. ANS is a dioxygenase that converts flavan-3-ols and leucoanthocyanidins to anthocyanidins. 3GT is a glycosyltransferase that adds a glucose group to the 3 OH group of anthocyanidins converting them to the corresponding anthocyanin 3-O-glucoside.

In some embodiments, the exogenous gene encoding for the tyrosine ammonia lyase (TAL) is a gene that encodes a polypeptide with at least 85%, 90%, or 95% amino acid sequence identity to any one of the TAL amino acid sequences identified herein.

In some embodiments, the exogenous gene encoding for the 4-coumaroyl-CoA ligase (4CL) is a gene that encodes a polypeptide with at least 85%, 90%, or 95% amino acid sequence identity to any one of the 4CL amino acid sequences identified herein.

In some embodiments, the exogenous gene encoding for the chalcone synthase (CHS) is a gene that encodes a polypeptide with at least 85%, 90%, or 95% amino acid sequence identity to any one of the CHS amino acid sequences identified herein.

In some embodiments, the exogenous gene encoding for the chalcone isomerase (CHI) is a gene that encodes a polypeptide with at least 85%, 90%, or 95% amino acid sequence identity to any one of the CHI amino acid sequences identified herein.

In some embodiments, the exogenous gene encoding for the malonyl-CoA synthetase (MatB) is a gene that encodes a polypeptide with at least 85%, 90%, or 95% amino acid sequence identity to any one of the MatB amino acid sequences identified herein.

In some embodiments, the exogenous gene encoding for the dicarboxylate carrier protein (MatC) is a gene that encodes a polypeptide with at least 85%, 90%, or 95% amino acid sequence identity to any one of the MatC amino acid sequences identified herein.

In some embodiments, the exogenous gene encoding for the flavanone 3β-hydroxylase (F3H) is a gene that encodes a polypeptide with at least 85%, 90%, or 95% amino acid sequence identity to any one of the F3H amino acid sequences identified herein.

In some embodiments, the exogenous gene encoding for the dihydroflavonol 4-reductase (DFR) is a gene that encodes a polypeptide with at least 85%, 90%, or 95% amino acid sequence identity to any one of the DFR amino acid sequences identified herein.

In some embodiments, the exogenous gene encoding for the leucoanthocyanidin reductase (LAR) is a gene that encodes a polypeptide with at least 85%, 90%, or 95% amino acid sequence identity to any one of the LAR amino acid sequences identified herein.

In some embodiments, the exogenous gene encoding for the anthocyanidin synthase (ANS) is a gene that encodes a polypeptide with at least 85%, 90%, or 95% amino acid sequence identity to any one of the ANS amino acid sequences identified herein.

In some embodiments, the exogenous gene encoding for the 3-glucosyl transferase (3GT) is a gene that encodes a polypeptide with at least 85%, 90%, or 95% amino acid sequence identity to any one of the 3GT amino acid sequences identified herein.

In all of the above instances of encoded polypeptides with at least 85%, 90%, or 95% amino acid sequence identity to a specified polypeptide, the function of the encoded polypeptide is the same as the function of the specified polypeptide. Those of skill in the art could readily determine amino acid sequences of such encoded polypeptides. Preservation of the function of the encoded polypeptide would be routine to a person of skill in the art with the benefit of the available information about functional domains of the specified polypeptides. Such information regarding domains includes disclosures in the references listed below, which are incorporated by reference in their entirety herein.
TAL: Crystal structure of phenylalanine ammonia lyase: multiple helix dipoles implicated in catalysis. Calabrese J C, Jordan D B, Boodhoo A, Sariaslani S, Vannelli T., Biochemistry. 2004 Sep. 14, 43(36):11403-16.
4CL: Identification of the substrate specificity-conferring amino acid residues of 4-coumarate:coenzyme A ligase allows the rational design of mutant enzymes with new catalytic properties, Stuible H P, Kombrink E., J Biol Chem. 2001 Jul. 20, 276(29):26893-7.
4CL: The substrate specificity-determining amino acid code of 4-coumarate: CoA ligase, Schneider K, Hovel K, Witzel K, Hamberger B, Schomburg D, Kombrink E, Stuible H P, Proc Natl Acad Sci USA. 2003 Jul. 8, 100(14):8601-6.
CHS: Structure of chalcone synthase and the molecular basis of plant polyketide biosynthesis, Ferrer J L, Jez J M, Bowman ME, Dixon R A, Noel J P., Nat Struct Biol. 1999 Aug. 6(8):775-84.
CHS: Dissection of malonyl-coenzyme A decarboxylation from polyketide formation in the reaction mechanism of a plant polyketide synthase, Jez J M, Ferrer J L, Bowman ME, Dixon R A, Noel J P., Biochemistry. 2000 Feb. 8, 39(5):890-902.
CHI: Structure and mechanism of the evolutionarily unique plant enzyme chalcone isomerase, Jez J M, Bowman M E, Dixon R A, Noel J P, Nat Struct Biol. 2000 Sep. 7(9):786-91.
DFR: Crystal structure of grape dihydroflavonol 4-reductase, a key enzyme in flavonoid biosynthesis, Petit P, Granier T, d'Estaintot B L, Manigand C, Bathany K, Schmitter J M, Lauvergeat V, Hamdi S, Gallois B., J Mol Biol. 2007 May 18, 368(5):1345-57.
LAR: Crystal structure and catalytic mechanism of leucoanthocyanidin reductase from Vitis vinifera., Mauge C, Granier T, d'Estaintot B L, Gargouri M, Manigand C, Schmitter J M, Chaudiere J, Gallois B., J Mol Biol., 2010 Apr. 9, 397(4):1079-91.
ANS: Structure and mechanism of anthocyanidin synthase from Arabidopsis thaliana, Wilmouth R C, Turnbull J J, Welford R W, Clifton U, Prescott A G, Schofield C J, Structure. 2002 Jan. 10(1):93-103.
3GT: Structural basis for acceptor-substrate recognition of UDP-glucose: anthocyanidin 3-O-glucosyltransferase from Clitoria ternatea, Hiromoto T, Honjo E, Noda N, Tamada T, Kazuma K, Suzuki M, Blaber M, Kuroki R., Protein Sci. 2015 Mar. 24(3):395-407. doi: 10.1002/pro.2630, PMID: 25556637.
3GT: Crystal structure of UDP-glucose: anthocyanidin 3-O-glucosyltransferase from Clitoria ternatea, Hiromoto T, Honjo E, Tamada T, Noda N, Kazuma K, Suzuki M, Kuroki R, J Synchrotron Radiat., 2013 Nov. 20(Pt 6):894-8.

In some embodiments, the method further includes: (a) when the microbial polyculture includes the TAL module cell, creating the TAL module cell by introducing an exogenous gene encoding for a tyrosine ammonia lyase (TAL) into a host cell for the TAL module cell; (b) when the microbial polyculture includes the C5 module cell, creating the C5module cell by introducing an exogenous gene encoding for a 4-coumaroyl-CoA ligase (4CL), an exogenous gene encoding for a chalcone synthase (CHS), an exogenous gene encoding for a chalcone isomerase (CHI), and, optionally, introducing an exogenous gene encoding for malonyl-CoA synthetase (MatB) and an exogenous gene encoding for putative dicarboxylate carrier protein (MatC), into a host cell for the C5 module cell; (c) when the microbial polyculture includes the p168 module cell, creating the p168 module cell by introducing an exogenous gene encoding for a flavanone 3β-hydroxylase (F3H), an exogenous gene encoding for a dihydroflavonol 4-reductase (DFR), and an exogenous gene encoding for a leucoanthocyanidin reductase (LAR) into a host cell for the p168 module cell; and (d) when the microbial polyculture includes the TAL module cell, creating the Antho module cell by introducing an exogenous gene encoding for an anthocyanidin synthase (ANS), and an exogenous gene encoding for a 3-glucosyl transferase (3GT) into a host cell for the Antho module cell.

In some embodiments, the host cells are E. coli cells. In one embodiment, the host cell for the TAL module cell is E. coli rpoA14(DE3). In one embodiment, the host cell for the C5 module cell is E. coli BL21star™(DE3)ΔsucCΔfumC. In one embodiment, the host cell for the p168 module cell is E. coli BL21star™(DE3). In one embodiment, the host cell for the Antho module cell is E. coli BL21star™(DE3).

The substrate of the TAL module cell is glucose, glycerol, or a combination thereof. The TAL module cell's product compound is a phenylpropanoic acid.

The substrate of the C5 module cell is a phenylpropanoic acid. When the C5module cell includes two additional genes, a gene for malonyl-CoA synthetase (MatB) and a gene for putative dicarboxylate carrier protein (MatC), the substrate of the C5 module cell is a phenylpropanoic acid, malonate, or a combination thereof. The C5 module cell's product compound is a flavanone.

The substrate of the p168 module cell is a flavanone. The p168 module cell's product compound is a flavonoid.

The substrate of the Antho module cell is a flavonoid. The Antho module cell's product compound is an anthocyanidin-3-O-glucoside.

The product compound is synthesized within a specified cell of the polyculture and is isolated from that cell, from the media, or from both the cell and the media.

In some embodiments, the phenylpropanoic acid is p-coumaric acid, caffeic acid, cinnamic acid, ferulic acid, or a combination thereof.

In some embodiments, the flavanone is naringenin, eriodictyol, pinocembrin, or a combination thereof.

In some embodiments, the flavonoid is a flavone, a flavan-3-ol, a flavan-4-ol, a flavonol, an anthocyanin, or a combination thereof.

In some embodiments, the flavone is luteolin, apigenin, chrysin, or a combination thereof.

In some embodiments, the flavan-3-ol is afzelechin, catechin (e.g., (+)-catechin), or a combination thereof.

In some embodiments, the flavan-4-ol is 4,5,7-trihydroxyflavan, 4,5,7,4′-tetrahydroxyflavan, 4,5,7,4′,5′-pentahydroxyflavan, 4,5,7,4′,5′,6′-hexahydroxyflavan, 4,5,7,4′-tetrahydroxy-5′methoxyflavan, or a combination thereof.

In some embodiments, the flavonol is kaempferol, quercetin, or a combination thereof.

In some embodiments, the anthocyanin is pelargonidin, cyanidin, delphinidin, malvidin, peonidin, or a combination thereof.

In some embodiments, the anthocyanidin-3-O-glucoside is pelargonidin-3-O-glucoside, cyanidin-3-O-glucoside, delphinidin-3-O-glucoside, malvidin-3-O-glucoside, peonidin-3-O-glucoside, or a combination thereof.

In some embodiments, the phenylpropanoic acid is p-coumaric acid and the flavonoid is (+)-afzelechin. In some embodiments, the phenylpropanoic acid is caffeic acid and the flavonoid is (+)-catechin. In some embodiments, the phenylpropanoic acid is cinnamic acid and the flavonoid is 3,5,7-trihydroxyflavan. In some embodiments, the substrate is glucose and the flavonoid is pelargonidin-3-O-glucoside.

In some embodiments, the substrate is glucose and the flavonoid is cyanidin-3-O-glucoside. In some embodiments, the substrate is glucose and the flavonoid is delphinidin-3-O-glucoside. In some embodiments, the substrate is glucose and the flavonoid is peonidin-3-O-glucoside. In some embodiments, the substrate is glucose and the flavonoid is malvidin-3-O-glucoside. In some embodiments, the substrate is glycerol and the flavonoid is cyanidin-3-O-glucoside. In some embodiments, the substrate is glycerol and the flavonoid is pelargonidin-3-O-glucoside. In some embodiments, the substrate is glycerol and the flavonoid is delphinidin-3-O-glucoside. In some embodiments, the substrate is glycerol and the flavonoid is peonidin-3-O-glucoside. In some embodiments, the substrate is glycerol and the flavonoid is malvidin-3-O-glucoside.

In some embodiments, the conditions permitting synthesis of the product compound comprise providing a carbon source to the microbial polyculture, wherein the carbon source is glucose, glycerol, xylose, arabinose, galactose, yeast extract, or a combination thereof. In some embodiments, the carbon source is any suitable pentose or hexose sugar.

The conditions permitting synthesis of the flavonoid compound may include an induction point, an induction temperature, and an inoculation ratio.

Thus, in one embodiment, the induction temperature is from about 10° C. to about 42° C. In one embodiment, the induction temperature of about 30° C. In one embodiment, the induction point is from about 0 hours to about 24 hours. In one embodiment, the induction point is at about 5.5 hours. In another embodiment, the induction point is at about 5 hours.

When each module cell is used as a plurality of cells, the inoculation ratio of the C5 module cell to the p168 module cell (C5:p168) is a ratio of from about 1:99 to about 99:1. In one embodiment, the inoculation ratio of the C5 module cell to the p168 module cell (C5:p168) is a ratio of about 8:2.

In one embodiment, the inoculation ratio of the TAL module cell to the C5 module cell to the p168 module cell to the Antho module cell (TAL:C5:p168:Antho) is a ratio of about 1-97:1-97:1-97:1-97. In one embodiment, the inoculation ratio of the TAL module cell to the C5 module cell to the p168 module cell to the Antho module cell (TAL:C5:p168:Antho) is a ratio of about 8:8:2:7.

In one embodiment, the inoculation ratio of the TAL module cell to the C5module cell (TAL:C5) is a ratio of about 1-99:1-99. In one embodiment, the inoculation ratio of the TAL module cell to the C5 module cell (TAL:C5) is a ratio of about 8:8.

In one embodiment, the inoculation ratio of the p168 module cell to the Antho module cell (p168:Antho) is a ratio of about 1-99:1-99. In one embodiment, the inoculation ratio of the p168 module cell to the Antho module cell (p168:Antho) is a ratio of about 2:7.

In one embodiment, the inoculation ratio of the TAL module cell to the C5 module cell to the p168 module cell (TAL:C5:p168) is a ratio of about 1-98:1-98:1-98. In one embodiment, the inoculation ratio of the TAL module cell to the C5 module cell to the p168 module cell (TAL:C5:p168) is a ratio of about 8:8:2.

In one embodiment, the inoculation ratio of the C5 module cell to the p168module cell to the Antho module cell (C5:p168:Antho) is a ratio of about 1-98:1-98:1-98. In one embodiment, the inoculation ratio of the C5 module cell to the p168 module cell to the Antho module cell (C5:p168:Antho) is a ratio of about 8:2:7.

The inoculation ratios are either volumentric ratios or ratios of the numbers of cells. When using volumetric ratios, cell concentrations are initially starting around 10 7 total cells/mL and increase to around 10 9 total cells/mL. These total cell counts can be split between the two or more strains in the polyculture.

The present invention is also directed to microbial polycultures of the above described methods. Thus, the invention includes a microbial polyculture that includes:
optionally, a TAL module cell including an exogenous gene encoding for a tyrosine ammonia lyase (TAL); optionally, a C5 module cell including an exogenous gene encoding for a 4-coumaroyl-CoA ligase (4CL), an exogenous gene encoding for a chalcone synthase (CHS), an exogenous gene encoding for a chalcone isomerase (CHI), and wherein, optionally, the C5 module cell further includes an exogenous gene encoding for malonyl-CoA synthetase (MatB) and an exogenous gene encoding for putative dicarboxylate carrier protein (MatC); optionally, a p168 module cell including an exogenous gene encoding for a flavanone 3β-hydroxylase (F3H), an exogenous gene encoding for a dihydroflavonol 4-reductase (DFR), and an exogenous gene encoding for a leucoanthocyanidin reductase (LAR); and optionally, an Antho module cell including an exogenous gene encoding for an anthocyanidin synthase (ANS), and an exogenous gene encoding for a 3-glucosyl transferase (3GT); with a proviso that:
the microbial polyculture includes the TAL module cell and the C5 module cell; or
the microbial polyculture includes the C5 module cell and the p168 module cell; or
the microbial polyculture includes the p168 module cell and the Antho module cell; or
the microbial polyculture includes the TAL module cell, the C5 module cell, and the p168module cell; or
the microbial polyculture includes the C5 module cell, the p168 module cell, and the Antho module cell; or
the microbial polyculture includes the TAL module cell, the C5 module cell, the p168 module cell, and the Antho module cell.

The invention is also directed to a method of producing a phenylpropanoic acid in a TAL module cell, wherein the TAL module cell is a microbial cell including an exogenous gene encoding for a tyrosine ammonia lyase (TAL); the method including: providing a substrate to the TAL module cell, wherein the substrate includes glucose, glycerol, or a combination thereof; culturing the TAL module cell under conditions permitting synthesis of the phenylpropanoic acid by the TAL module cell; and isolating the phenylpropanoic acid synthesized by the TAL module cell. In one embodiment, the method further includes creating the TAL module cell by introducing an exogenous gene encoding for a tyrosine ammonia lyase (TAL) into a host cell for the TAL module cell.

In one embodiment, the exogenous gene encoding for the tyrosine ammonia lyase (TAL) is a gene encoding for Rhodotorula glutinis tyrosine ammonia lyase (RgTAL). In some embodiments, the exogenous gene encoding for the tyrosine ammonia lyase (TAL) is a gene encoding for Rhodotorula glutinis tyrosine ammonia lyase (RgTAL), Rhodobacter capsulatus TAL, Rice TAL, Parsley TAL, Tomato TAL, Arabidopsis TAL, or a combination thereof.

In some embodiments, the phenylpropanoic acid is p-coumaric acid, caffeic acid, cinnamic acid, ferulic acid, or a combination thereof, all of which could derive from glucose substrate.

In some embodiments, the host cell for the TAL module cell is E. coli rpoA14(DE3).

The invention is also directed to a TAL module cell, wherein the TAL module cell is a microbial cell including an exogenous gene encoding for a tyrosine ammonia lyase (TAL). In one embodiment, the exogenous gene encoding for the tyrosine ammonia lyase (TAL) is a gene encoding for Rhodotorula glutinis tyrosine ammonia lyase (RgTAL). In one embodiment, a host cell for the TAL module cell is E. coli, for example, E. coli rpoA14(DE3).

In general, the terms and phrases used herein have their art-recognized meaning, which can be found by reference to standard texts, journal references and contexts known to those skilled in the art. The following definitions are provided to clarify their specific use in the context of the invention.

The following nucleic acid name abbreviations are used herein: C or c for cytosine, G or g for guanine, A or a for adenine, T or t for Thymine, and U or u for uracil.

The following amino acid name abbreviations are used herein: A or Ala for Alanine; M or Met for Methionine; C or Cys for Cysteine; D or Asp for Aspartic Acid; E or Glu for Glutamic Acid; F or Phe for Phenylalanine; G or Gly for Glycine; H or His for Histidine; I or Ile for Isoleucine; K or Lys for Lysine; L or Leu for Leucine; N or Asn for Asparagine; P or Pro for Proline; Q or Glu for Glutamine; R or Arg for Arginine; S or Ser for Serine; T or Thr for Threonine; V or Val for Valine; W or Trp for Tryptophan; and Y or Tyr for Tyrosine.

The terms “microbe” and “microbial” refer to a microscopic living organism, which may be single-celled or multicellular. Microbe, as used herein, includes bacteria, unicellular eukaryotes, archaea, and protozoa. An example of a microbe used in the inventions described herein is E. coli.

The term “isolating the product compound”, as used herein, encompases any method that increases purity of the product compound.

When a reference is made to a gene that encodes for a specified polypeptide, such gene has the meaning of any nucleic acid sequence that encodes for the amino acid sequence of the specified polypeptide. Those of skill in the art could readily determine all possible nucleic acid sequences encoding for the specified polypeptide.

The term “induction point”, as used herein, refers to the time point, after the culture has been initiated, at which the inducer is added to the medium.

The term “induction temperature”, as used herein, refers to the temperature at which the culture is left to grow after the inducer has been added into the medium.

The following specific non-limiting examples are illustrative of the invention. Examples 1-14 describe studies that are also described in more detail in Jones, J. A. et al. Experimental and computational optimization of an Escherichia coli co-culture for the efficient production of flavonoids. Metab. Eng. 35, 55-63 (2016), entire disclosure of which, together with accompanying supplementary data available online at http://dx.doi.org/10.1016/j.ymben.2016.01.006, is incorporated by reference in its entirety.

### EXAMPLE 1

### C5 Module and p168 Module Polyculture—Bacterial Strains, Vectors, and Media

E. coli DH5α was used to propagate all plasmids, while the BL21star™(DE3), BL21star™(DE3)ΔsucCΔfumC, or BL21star™(DE3)ΔpgiΔppc was used as the hosts for flavonoid production. The ePathBrick vector, pETM6, was used as the basis for all plasmid construction and pathway expression. Luria Broth (LB) Lennox modification (Sigma) and Andrew's Magic Medium (AMM) were used where noted. Sequences of all plasmid constructs are available through Addgene.org and are incorporated by reference herein.

### EXAMPLE 2

### C5 Module and p168 Module Polyculture—Flavonoid Pathways and ePathOptimize Library Construction

Genes involved in the 12 candidate upstream flavanone production pathways were obtained from previously published literature from the Koffas lab. Vv4CL, Pc4CL, CmCHS, PhCHS, CmCHI, and MsCHI were obtained in ePathBrick vector pETM6, while At4CL was acquired through PCR amplification (ACCUZYME 2.times. mix, Bioline) of plasmid #3 DNA using primers 1 and 2 (Table 2). The ePathBrick destination vector, pETM6, and At4CL PCR amplicon were digested with restriction enzymes NdeI/XhoI (FastDigest, Thermo Scientific) and gel purified (E.Z.N.A. MicroElute Gel Extraction Kit, Omega Bio-tek). Digested At4CL PCR product was ligated with digested pETM6 backbone to create plasmid 2, Table 1. Constructs were then transformed into chemically competent DH5α for verification and plasmid propagation. Colonies were screened via restriction digest and further verified with Sanger sequencing (GENEWIZ, Inc.) using the sequencing primers 3 and 4 in Table 2. Site directed mutagenesis was then preformed using standard protocols to silently remove the NheI restriction site from At4CL using primers 5 and 6 (Table 2). Complete candidate pathways were constructed in monocistronic form using standard ePathBrick methods resulting in plasmids 10-27, Table 1. Occasionally the restriction site ApaI was used to replace SalI when the pathway genes either contained internal SalI restriction sites or to optimize the insert: backbone ratio for improved ligation efficiency. Plasmids p148 and p168 containing complete downstream modules were not modified from previous reports.

TABLE 1
List of Strains and Plasmids

| Number | Strain or vector | Relevant properties | Reference |
|---|---|---|---|
| S1 | Escherichia coli | F⁻, φ80d lacZΔM15, Δ(lacZYA-argF)U169, | Novagen |
| | DH5α | recA1, endA1, hsdR17(rk⁻, mk⁺), phoA, | |
| | | supE44λ⁻, thi⁻¹, gyrA96, relA1 | |
| S2 | E. coli BL21 Star ™ | F⁻ompT gal dcm rne131 lon hsdS_{B} (r_{B}⁻m_{B}⁻) | Invitrogen |
| | (DE3) | λ(DE3) | |
| S3 | BLΔpgiΔppc | BL21Star ™ (DE3)Δpgi::FRTΔppc::FRT- | (a) |
| | | KanR-FRT | |
| S4 | BLΔsumCΔfumC | BL21Star ™ (DE3)ΔfumC::FRTΔsucC::FRT | (b) |
| 1 | pETM6 | ePathBrick expression vector, ColE1 ori, | (c) |
| | | AmpR | |
| 2 | pETM6-At4CL | #1 with 4CL-1 from A. thaliana | This Study |
| 3 | pC-At4cl-Vvsts | pCDFDuet with 4CL-1 from A. thaliana, STS | (d) |
| | | from V. vinifera | |
| 4 | pETM6-Pc4CL | #1 with 4CL-2 from P. crispum | (c) |
| 5 | pETM6-Vv4CL | #1 with 4CL from V. vinifera | (e) |
| 6 | pETM6-PhCHS | #1 with CHS from P. hybrida | (c) |
| 7 | pETM6-CmCHS | #1 with CHS from C. maxima | (e) |
| 8 | pETM6-MsCHI | #1 with CHI from M. sativa | (c) |
| 9 | pETM6-CmCHI | #1 with CHI from C. maxima | (e) |
| 10 | pETM6-At4CL- | #1 with At4CL and PhCHS, monocistronic | This Study |
| | PhCHS | form | |
| 11 | pETM6-At4CL- | #1 with At4CL and CmCHS, monocistronic | This Study |
| | CmCHS | form | |
| 12 | pETM6-Pc4CL- | #1 with Pc4CL and PhCHS, monocistronic | This Study |
| | PhCHS | form | |
| 13 | pETM6-Pc4CL- | #1 with Pc4CL and CmCHS, monocistronic | This Study |
| | CmCHS | form | |
| 14 | pETM6-Vv4CL- | #1 with Vv4CL and PhCHS, monocistronic | This Study |
| | PhCHS | form | |
| 15 | pETM6-Vv4CL- | #1 with Vv4CL and CmCHS, monocistronic | This Study |
| | CmCHS | form | |
| 16 | pETM6-At4CL- | #1 with At4CL, PhCHS, and MsCHI, | This Study |
| | PhCHS-MsCHI | monocistronic form | |
| 17 | pETM6-At4CL- | #1 with At4CL, PhCHS, and CmCHI, | This Study |
| | PhCHS-CmCHI | monocistronic form | |
| 18 | pETM6-At4CL- | #1 with At4CL, CmCHS, and MsCHI, | This Study |
| | CmCHS-MsCHI | monocistronic form | |
| 19 | pETM6-At4CL- | #1 with At4CL, CmCHS, and CmCHI, | This Study |
| | CmCHS-CmCHI | monocistronic form | |
| 20 | pETM6-Pc4CL- | #1 with Pc4CL, PhCHS, and MsCHI, | This Study |
| | PhCHS-MsCHI | monocistronic form | |
| 21 | pETM6-Pc4CL- | #1 with Pc4CL, PhCHS, and CmCHI, | This Study |
| | PhCHS-CmCHI | monocistronic form | |
| 22 | pETM6-Pc4CL- | #1 with Pc4CL, CmCHS, and MsCHI, | This Study |
| | CmCHS-MsCHI | monocistronic form | |
| 23 | pETM6-Pc4CL- | #1 with Pc4CL, CmCHS, and CmCHI, | This Study |
| | CmCHS-CmCHI | monocistronic form | |
| 24 | pETM6-Vv4CL- | #1 with Vv4CL, PhCHS, and MsCHI, | This Study |
| | PhCHS-MsCHI | monocistronic form | |
| 25 | pETM6-Vv4CL- | #1 with Vv4CL, PhCHS, and CmCHI, | This Study |
| | PhCHS-CmCHI | monocistronic form | |
| 26 | pETM6-Vv4CL- | #1 with Vv4CL, CmCHS, and MsCHI, | This Study |
| | CmCHS-MsCHI | monocistronic form | |
| 27 | pETM6-Vv4CL- | #1 with Vv4CL, CmCHS, and CmCHI, | This Study |
| | CmCHS-CmCHI | monocistronic form | |
| 28 | p148 | #1 with CsF3H^{syn}-AaDFR^{syn}-DuLAR^{syn}, | (f) |
| | | monocistronic form | |
| 29 | p168 | #1 with CsF3H^{syn}-FaDFR^{syn}-DuLAR^{syn}, | (f) |
| | | monocistronic form | |
| 30 | pETM6-mCherry | #1 with mCherry fluoroscent reporter | (c) |
| 31 | pETM6-C4- | #30 Modified with mutated ‘C4’ T7 promoter | (g) |
| | mCherry | sequence | |
| 32 | pETM6-G6- | #30 Modified with mutated ‘G6’ T7 promoter | (g) |
| | mCherry | sequence | |
| 33 | pETM6-H9- | #30 Modified with mutated ‘H9’ T7 promoter | (g) |
| | mCherry | sequence | |
| 34 | pETM6-H10- | #30 Modified with mutated ‘H10’ T7 | (g) |
| | mCherry | promoter sequence | |
| 35 | pFlavo^{opt} or C5 | #17 with C4 mutant T7 promoter controlling | This Study |
| | mutant | CmCHI | |

TABLE 2
List of Plasmids

| Number | Primer ID | Primer Sequence (5′→3′) |
|---|---|---|
| 1 | At4CL_FWD with NdeI | GCGCCGCATATGGCGCCACAAGA |
| (SEQ ID NO: 1) | | ACAAG |
| 2 | At4CL_REV with XhoI | GCGCGGCTCGAGTCACAATCCATT |
| (SEQ ID NO: 2) | | TGCT |
| 3 | Seq_T7_FWD | TAATACGACTCACTATAGGG |
| (SEQ ID NO: 3) | | |
| 4 | Seq_T7Term_REV | GCTAGTTATTGCTCAGCGG |
| (SEQ ID NO: 4) | | |
| 5 | SDM_At4CL_NheI_FWD | GAATGACGGAAGCAGGTCCAGTG |
| (SEQ ID NO: 5) | | CTCGCAATGTCGTTAGGTTTTGCA |
| | | AAG |
| 6 | SDM_At4CL_NheI_REV | CTTTGCAAAACCTAACGACATTGC |
| (SEQ ID NO: 6) | | GAGCACTGGACCTGCTTCCGTCAT |
| | | TC |

The upstream pathway genes were cloned in monocistronic form with randomized promoter strengths using previously published methods. Multiple transformations were oftentimes completed to ensure sufficient library sampling and retention. The final plasmid library, pETM6-xxAt4CL-xxPhCHS-xxCmCHI, was transformed into BL21star™(DE3)ΔsucCΔfumC for screening. The ‘xx’ feature represents the inclusion of a single random mutant T7 promoter from the five-member ePathOptimize library.

### EXAMPLE 3

### C5 Module and p168 Module Polyculture—Small-Scale Cultivation Protocol

Single colonies of each strain were inoculated separately into 25 mL of AMM in a 125 mL non-baffled shake flask with ampicillin (80 μg/mL) and grown overnight at 37° C. After 14 hours, the overnight cultures were mixed volumetrically to the indicated inoculation ratios and were inoculated at 2% (40 μL) into 2 mL of AMM and allowed to grow at 37° C. before induction with 1 mM IPTG. Upon induction, the cultures were transferred to the appropriate induction temperature and grown for 48 hours. All small-scale screening was completed in polypropylene 48-well plates (5 mL, VWR). Except where noted, the cultures were grown in AMM with 20 g/L Glycerol, 100 mg/L of substrate was added at induction, and 30° C. was used as the induction temperature.

### EXAMPLE 4

### C5 Module and p168 Module Polyculture—Bioreactor Fermentation Protocol

Fed-batch style fermentation was performed using a DASGIP parallel bioreactor at an initial working volume of 500 mL of AMM with 20 g/L glycerol as a carbon source. Overnight cultures were prepared identically to the small-scale protocol presented above. The bioreactor was inoculated at an initial ratio of 7:3 (C5:p168) at 2% of final volume. The pH and DO of the fermentation broth was maintained at 7.2 and 50 percent saturation through addition of 6M sodium hydroxide and application of stirring cascade control, respectively. The feed solution [250 g/L glycerol, 4 g/L casamino acids, 7 g/L (NH₄)₂HPO₄, and 80 μg/mL ampicillin] and 2.times.MOPS mix was fed at 2 mL per hour from 5-15 hours and 4 mL per hour from 15-26 hours. The fermentation was induced with IPTG to a final concentration of 1 mM after 7 hours of growth (OD₆₀₀=7.1) and the system was cooled to 30° C. The substrate, p-coumaric acid, was added in 50 mg/L aliquots at 1, 4, and 7 hours post induction. Samples were taken periodically for measurement of OD₆₀₀ and metabolite analysis.

### EXAMPLE 5

### C5 Module and p168 Module Polyculture—Metabolite Analysis

Fermentation broth was mixed with an equal volume of absolute ethanol and vortexed for 10 seconds prior to centrifugation (10 min, 20,000.times.g). The supernatant (25 μL) was used for HPLC analysis carried out using Agilent 1200 series HPLC equipped with a ZORBAX SB-18 column (5 μm, 4.6. times. 150 mm) and a diode array detector. The mobile phase was acetonitrile (solvent A) and water (solvent B) (both contain 0.1% formic acid) at a flow rate of 1 mL/min. HPLC program was as follows: 10 to 40% A (0-10 min) and 40 to 60% A (10-15 min). Absorbance at 280 nm was monitored in all cases. Titer of products was determined using authentic standards while (+)-afzelechin was quantified using the (+)-catechin calibration curve. All experiments were performed in duplicate. Error bars represent ±1 standard deviation of biological duplicate. Significance of data was determined using a two-tailed unpaired t-test with a 95 percent confidence interval.

### EXAMPLE 6

### C5 Module and p168 Module Polyculture—Empirical Modeling Methods

Experimental conditions were modeled using empirical modeling methods, which are described in detail in Jones, J. A. et al. Experimental and computational optimization of an Escherichia coli co-culture for the efficient production of flavonoids. Metab. Eng. 35, 55-63 (2016).

### EXAMPLE 7

### C5 Module and p168 Module Polyculture

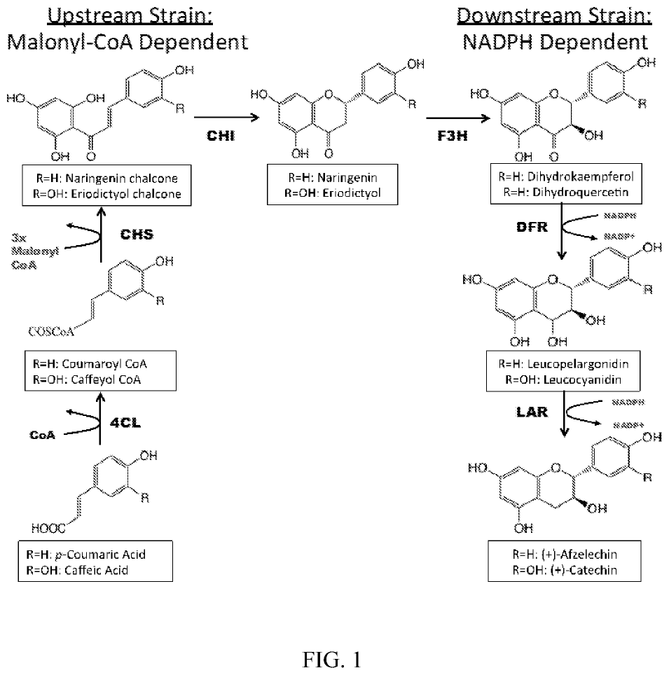

The production of flavan-3-ols from phenylpropanoic acid precursors proceeded through six enzymatic steps: 4-coumaroyl-CoA ligase, 4CL; chalcone synthase, CHS; chalcone isomerase, CHI; flavanone 3β-hydroxylase, F3H; dihydroflavonol 4-reductase, DFR; leucoanthocyanidin reductase, LAR; (FIG. 1). The complete pathway was partitioned such that both the upstream and downstream modules contained three genes. This modularization reduced the metabolic burden of enzyme overexpression and divided the pathway according to necessary co-factor requirements: malonyl-CoA (upstream) and NADPH (downstream).

### EXAMPLE 8

### C5 Module and p168 Module Polyculture—Independent Optimization of Upstream and Downstream Modules

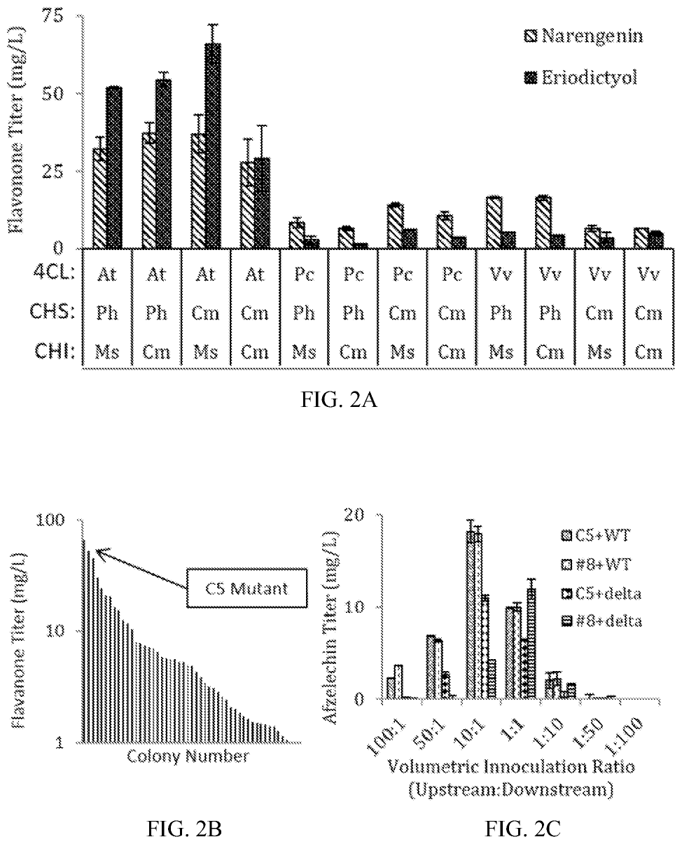

The ability to tailor the genetic optimization of each strain in a co-culture system for improved flux towards necessary co-factors and substrates through the pathway of interest and away from unwanted side products is a major advantage over monoculture methods. We began our modular optimization by focusing on the upstream strain containing 4CL, CHS, and CHI. Building on previous efforts to optimize malonyl-CoA availability, BL21star™(DE3)ΔsucCΔfumC was chosen as the host strain for this upstream module. We then chose homologs for each of the three enzymes from different plant sources, resulting in 12 combinations of potential upstream pathways. Upon screening for functional conversion of two phenylpropanoic acid precursors to their corresponding flavanones, several high-titer homolog combinations were discovered (FIG. 2A). Constructs containing the 4CL from Arabidopsis thaliana (At4CL) showed significantly (p<0.001) higher conversion leading towards the choice of construct containing At4CL, PhCHS, and CmCHI for further optimization.

Using the recently published ePathOptimize technique for modulating the transcriptional landscape, the promoter strengths of each gene in the upstream module were randomized to one of five mutant T7 promoters of various strength. The library members were then screened for conversion of p-coumaric acid to naringenin in vivo (FIG. 2B). The results indicated high sensitivity to promoter strength and resulted in one mutant (C5 or pFlavo^{opt}) that out-performed the consensus T7 control strain by 24 percent. This pFlavo^{opt} mutant was sequenced and was found to have the consensus T7 sequence controlling expression of At4CL and PhCHS, while the strong mutant promoter C4 was found to control expression of CmCHI. The nomenclature C5 or pFlavo^{opt} refers to the transcriptionally optimized plasmid expressed in the flavanone expression strain BL21star™(DE3) (Table 1) and contains the incorporation of ePathOptimize mutant T7 promoter C4 controlling the expression of CmCHI. This transcriptionally optimized plasmid was then utilized in future co-cultures.

Optimization of the downstream pathway has been previously explored through screening of 18 homolog gene combinations resulting in two combinations that exhibit efficient conversion of both naringenin and eriodictyol substrates across a wide range of substrate concentrations (67 Zhao et al., 2015). To confirm the findings of this previous study, both the p148 and p168 constructs were tested using a cultivation protocol and substrate concentration realistic to the levels expected in the current study. Similar titers and trends were obtained with p168 slightly out-performing p148, leading towards the choice of p168 for the downstream module in the co-culture optimization. Further optimization of plasmid p168 was not performed due to limiting fluxes through the upstream module. With independent genetic optimization of the upstream and downstream modules completed, the lead candidates for each module were then screened for strain compatibility in co-culture.

### EXAMPLE 9

### C5 Module and p168 Module Polyculture—Determination of Co-Culture Compatibility

Strain compatibility is a significant factor in any co-culture system. The strains must be able to efficiently grow in the same media, have the same antibiotic selection, and must not produce toxic compounds that significantly harm the other members of the microbial community. Many of these criteria can be easily addressed by using strains of similar background, but module specific mutations towards improving intercellular conditions for the pathway of interest can impact cellular compatibility in co-culture. Furthermore, pathway metabolites that connect the individual members of the co-culture must be readily transferred across the cell membrane from the producer to the consumer.

Two strains from each the upstream and downstream module were tested for their cross compatibility in co-culture. For the upstream strain, the transcriptionally optimized pFlavo^{opt} mutant and the consensus control plasmid (#17, Table 1) were used in strain BL21star™(DE3)ΔsucCΔfumC, while for the downstream module a single plasmid, p168, was tested in two host strains: wild type BL21star™(DE3) and BL21star™(DE3)ΔpgiΔppc. We have noticed a significant decrease in cell growth for the ΔpgiΔppc strain background and hypothesized that this would affect strain performance in co-culture. Four co-culture combinations were tested across various initial inoculation cell ratios (FIG. 2C) and a significant reduction in flavan-3-ol titer was seen for the two co-cultures containing BL21*(DE3)ΔpgiΔppc (p<0.001). Nearly identical performance was achieved by strains containing either the consensus control or the pFlavo^{opt} mutant upstream module. From these results, we chose BL21*(DE3)ΔsucCΔfumC with the pFlavo^{opt} mutant upstream module and the wild type BL21*(DE3) with the p168 plasmid for further optimization.

### EXAMPLE 10

### C5 Module and p168 Module Polyculture—Determination of Important Optimization Parameters

To begin fermentation optimization of the co-culture system, we identified two key parameters predicted to result in high sensitivity: induction point and inoculation ratio. Both the upstream and downstream modules contain pET expression cassettes controlled by the T7-lac system, and therefore protein production is inducible with the addition of Isopropyl β-D-1-thiogalactopyranoside (“IPTG”). A wide variety of optimum induction points have been presented in the primary literature for pET-based systems indicating that the optimum induction point is linked to division of cellular resources and is more complex than purely affecting protein production levels. Due to this complexity, the optimum induction point is specific to the particular system and set of cultivation conditions and must be determined experimentally.

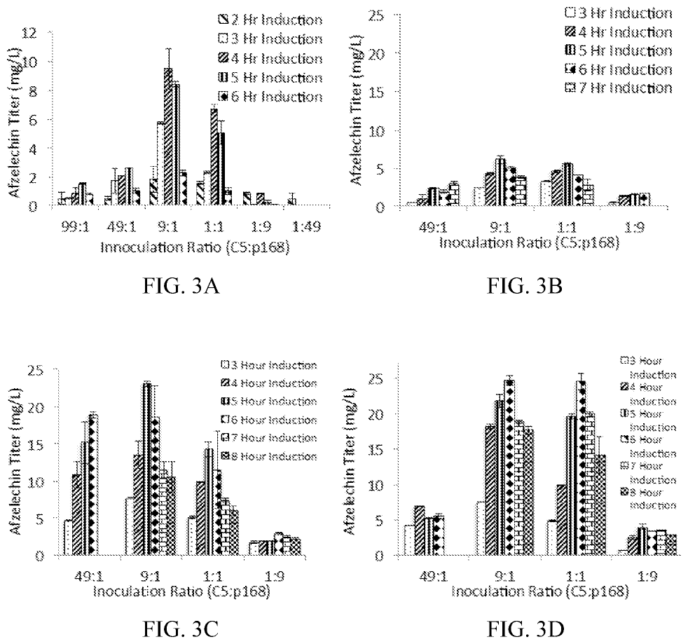

The initial inoculation ratio of upstream to downstream cells in the fermentation is another important parameter that adds to the complexity of co-culture systems. Variation of this ratio allows for changes to be made in population dynamics, accounting for differences in population growth rate and specific activity of the strains in co-culture. Interestingly, when various induction points were crossed with multiple inoculation ratios, we saw an orthogonal response in product titer from the two parameters (FIG. 3A). The system demonstrated a peak induction point of 4 hours post-inoculation regardless of inoculation ratio and a peak inoculation ratio of 9:1 regardless of induction point, resulting in the point of highest titer at a 4-hour induction and an initial inoculation ratio of 9:1 (C5:p168). This finding led to the decision to screen all future parameters across various induction points and inoculation ratios to visualize the production landscape. Furthermore, the observed trends indicate that the system is stable over a wide range of initial inoculation ratios, showing no tipping point where one strain demonstrates a propensity to dominate the population with time. Additional analysis of substrate and flavanone intermediate concentrations also vary as expected with variable inoculation ratio. In co-cultures with dominant upstream ratios, considerable initial substrate is utilized and intermediate product is accumulated, but little intermediate is converted to final product; while co-cultures with dominant downstream ratios utilized little initial substrate, limiting flux through the entire system. However, at intermediate inoculation ratios, high amounts of initial substrate are utilized while low intermediate product titers are present due to efficient conversion to the final product.

### EXAMPLE 11

### C5 Module and p168 Module Polyculture—Effect of Carbon Source

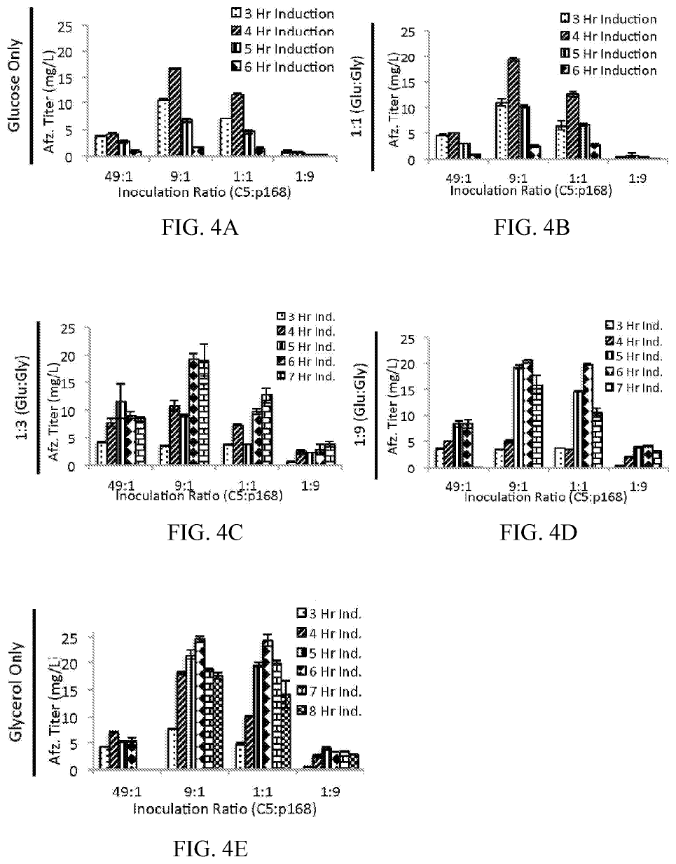

Previous literature reports and early experimental evidence (data not shown) fueled the decision to use the Andrew's Magic Medium (AMM) with 20 g/L of glucose as the initial production media for individual strain optimization and preliminary co-culture experiments. In an attempt to reduce the production costs at the industrial scale, and because of the increased interest to utilize glycerol for industrial fermentations, we varied the proportion of glucose to glycerol in the culture media. In addition to economic incentives, the preference for glycerol over other carbon sources has been reported for different microbial strains due to strain-specific differences in gene expression and metabolite profiles upon growth on glycerol. With all media having 20 g/L total carbon source, five carbon source ratios were tested ranging from glucose only to glycerol only (FIG. 4A-E). Several trends in the production landscape were observed upon the shift from growth on glucose to glycerol. The most noticeable trend was higher optimum titers with increasing proportion of glycerol. Upon growth on increasing proportions of glycerol, a shift in the production landscape resulted in higher titers appearing at later induction points and peak inoculation ratios with higher proportion of the downstream strain. Additionally, glucose-grown cultures demonstrate a sharp peak in the production landscape, where glycerol-grown cultures show a plateau with many high-titer solutions.

### Example 12

### C5 Module and p168 Module Polyculture—Induction Temperature Optimization

Fermentation temperature can affect cellular growth dynamics, enzyme folding, and specific enzyme activity. These effects have not been well documented on the systems level, such that optimum fermentation temperature could be predicted for any given system a priori. We therefore decided to test co-culture production at induction temperatures of 10, 20, or 30° C. The co-culture was grown at 37° C. prior to induction at which the temperature was then dropped to the specified induction temperature after induction. Previous efforts have maintained an induction temperature of 30° C. A significant decrease in optimal titer was observed in the 10° C. case with the 20 and 30° C. cases showing similar maximum achieved titers (FIG. 3B-D). Although similar in optimum titer, the 20 and 30° C. cases did show different production landscapes such that the 20° C. case had a sharp optimum while the 30° C. case demonstrated more of a plateau with many conditions resulting in moderately high titers. Additionally, similar trends were observed for increasing induction temperature as were seen for increasing proportion of glycerol in the media. Notably, increases in induction temperature resulted in a shift of the production landscape towards optimum solutions with later induction points and inoculation ratios favoring more of the downstream strain.

### EXAMPLE 13

### C5 Module and p168 Module Polyculture—System Modeling for Prediction of Optimum Operating Conditions

The aforementioned observations suggested that the titer achieved by the system could be improved by selecting optimized experimental conditions. To identify potential conditions that could result in an optimal titer, an empirical modeling approach was utilized. Due to the trends observed from preliminary data showing the dependence of titer on induction point, inoculation ratio, carbon source, and induction temperature, we constructed an empirical scaled-Gaussian model, which uses these four experimental variables as inputs and computes the titer. This model contains 21 parameters that were fitted using 72 experimental data points. In particular, titer was measured at each combination of the following: induction point—3, 4, 5, 6 hours; inoculation ratio (upstream: downstream)—49:1, 9:1, 1:1; carbon source (glucose: glycerol)—1:0, 1:1, 0:1; induction temperature—20, 30° C. The model demonstrates a close fit with the training data, and follows the general trend of additional data that were not used for model fitting. The optimal point of the model function was determined computationally, and was used to direct future experiments in search of optimal operating conditions to maximize titer. Interestingly, the optimal point of the model function was found to be at operating conditions not tested previously, and within a gap between previously tested experimental points. Specifically, the optimal conditions predicted by the model were: induction point of 5.5 hours; inoculation ratio of 7:3 (upstream: downstream); carbon source ratio of 0:1 (glucose: glycerol); and induction temperature of 25° C.

Experiments were subsequently performed at conditions in the region of the model-predicted optimum. These experiments resulted in a maximum titer of 40.7±0.1 mg/L, a 65% increase over the highest titer measured prior to computational optimization. This maximal titer was achieved experimentally at an induction point of 6 hours; inoculation ratio of 8:2 (upstream: downstream); carbon source ratio of 0:1 (glucose: glycerol); and induction temperature of 30° C. This point was within the set of experimental points we tested based on proximity with the model-predicted optimum, but the point differs slightly from the model-predicted optimum. This is not surprising, as a scaled-Gaussian model was used for fitting the data and computing the optimum, whereas the behavior of the true system is likely more complex than can be fully captured by such an empirical model. That being said, using a scaled-Gaussian model represented a good trade-off between model complexity and quality of fit for the available data, and the model was ultimately successful in guiding experiments to achieve substantially higher titers. This suggests that relatively simple empirical models can be effective tools for informing titer optimization efforts.

### EXAMPLE 14

### C5 Module and p168 Module Polyculture—Bioreactor Scale-Up: Proof of Principle

To demonstrate the stability and scalability of our co-culture system, we showed scale-up of the fermentation from a 2 mL culture in a 48-well plate directly to a bioreactor with a 500 mL working volume. Utilizing near optimum conditions from previous small-scale optimization experiments, the bioreactor demonstrated slightly lower (34 vs. 41 mg/L) product titers than that of the optimized small-scale system. We predict this is due to a shift in the production landscape as a result of scale-up but believe that global trends due to induction point, inoculation ratio, media composition, and induction temperature will remain constant for the system. The additional control gained through the use of bioreactors also results in additional complexity from a pathway optimization standpoint. To that end, the complete fermentation optimization of our co-culture system is beyond the scope of this work but represents a promising direction for future optimization studies.

The ability to harness the power of multiple strains in co-culture allows for a division of metabolic burden across the population, as well as the ability to genetically optimize each module individually for specific co-factor and precursor requirements. Through exploitation of these advantages and empirical modeling techniques, we were able to improve production of flavonoids to 40.7±0.1 mg/L, a 970-fold improvement over previous monoculture efforts.

### EXAMPLE 15

### TAL Module, C5 Module, p168 Module, and Antho Module Polyculture—Bacterial Strains, Vectors, and Media

E. coli DH5α was used to propagate all plasmids, while BL21star™(DE3), BL21star™(DE3)ΔsucCΔfumC, rpoA14(DE3), or QH4 was used as the hosts for flavonoid production. The expression vectors, pETM6 or pXPA, were the basis for all plasmid construction and pathway expression. Luria Broth (LB) Lennox modification (Sigma) and Andrew's Magic Media (AMM) were used where noted. Sequences of all plasmid constructs are available through addgene.org and are incorporated by reference herein.

### EXAMPLE 16

### TAL Module, C5 Module, p168 Module, and Antho Module Polyculture—Plasmid Construction

Many preexisting flavonoid modules were used directly or slightly modified for this work. All plasmids used are summarized in Table 3 and all plasmid modifications are described below. Site directed mutagenesis was performed to silently remove an internal NdeI restriction site from the open reading from of Rhodotorula glutinis Tyrosine Ammonia Lyase (RgTAL^{syn}) on pTrc-RgTAL^{syn} using standard methods and primers 13-14, Table 4. The mutagenized RgTAL^{syn} was PCR amplified from pTrc-RgTAL^{syn} using primers 11-12, Table 4. The resulting PCR product was digested (FastDigest, Thermo Scientific) with NdeI and SpeI, gel purified (E.Z.N.A MicroElute Gel Extraction Kit, Omega Bio-tek), and ligated with pETM6 backbone also digested with NdeI and SpeI and gel extracted corresponding to standard methods to create pETM6-RgTAL^{syn}, (#10, Table 3). The corresponding plasmid was sequence verified (GENEWIZ, Inc.) and used together with pETM6-HpaBC (#12, Table 3) to create pETM6-RgTALsyn-HpaB-HpaC via standard ePathBrick cloning protocols.

To create the constitutive expression plasmid, pXy1A, we replaced the T7-lac feature on pETM6 with the P_{xy1A} promoter from Bacillus megaterium found on the commercial vector, pMM1522 (Mobitec). To this end, a gBlock (Integrated DNA Technologies, sequence provided in Table 5) was synthesized containing the MCS of pETM6 under the control of the P_{xy1A} promoter sequence, flanked by AvrII and SpeI restriction sites on the 5′ and 3′ ends, respectively. The P_{xy1A} fragment was then cloned into pETM6 and sequence verified. Two constitutive TAL expression plasmids were obtained by sub-cloning RgTAL^{syn} from pETM6-RgTAL^{syn} into pXy1A and pXPA-eGFP (P_{GAP} promoter) at restriction sites Ndel and SpeI using standard methods.

TABLE 3
Strains and plasmids used in this study.

| Number | Strain or vector | Relevant properties | Reference |
|---|---|---|---|
| S1 | Escherichia coli | F⁻, φ80d lacZΔM15, Δ(lacZYA- | Novagen |
| | DH5α | argF)U169, recA1, endA1, | |
| | | hsdR17(rk⁻, mk⁺), phoA, supE44λ⁻, | |
| | | thi⁻¹, gyrA96, relA1 | |
| S2 | E. coli BL21 Star ™ | F⁻ompT gal dcm rne131 lon hsdS_{B} | Invitrogen |
| | (DE3) | (r_{B}⁻m_{B}⁻) λ(DE3) | |
| S3 | BLΔpgiΔppc | BL21Star ™ (DE3)Δpgi::FRTΔppc::FRT- | 30 |
| | | KanR-FRT | |
| S4 | BLΔsumCΔfumC | BL21Star ™ (DE3)ΔfumC::FRTΔsucC::FRT | 31 |
| S5 | rpoA14(DE3) | E. coli K12 ΔpheA ΔtyrR lacZ::P_{LtetO-1}- | 16 |
| | | tyrA^{fbr}aroG^{fbr}tyrR::P_{LtetO-1}- | |
| | | tyrA^{fbr}aroG^{fbr} hisH(L82R) pHACM- | |
| | | rpoA14, λ(DE3) | |
| S6 | QH4 | E. coli ATCC 31884/ΔpheLA-tyrA | 21 |
| 1 | pETM6 | ePathBrick expression vector, ColE1 | 18 |
| | | ori, AmpR | |
| 2 | p168 | #1 with CsF3H^{syn}-FaDFR^{syn}- | 32 |
| | | DuLAR^{syn}, monocistronic form | |
| 3 | pETM6-mCherry | #1 with mCherry fluoroscent reporter | 18 |
| 4 | pFlavo^{opt} or C5 | #17 with C4 mutant T7 promoter | 9 |
| | mutant | controlling CmCHI | |
| 5 | pTrc-RgTAL^{syn} | pTrcHis2B carrying codon- | 16 |
| | | optimized R. glutinis TAL | |
| 6 | pCS-TPTA | From pCS27, P_{L}lacO1; tyrA^{fbr}-ppsA- | 22 |
| | | tktA-aroG^{fbr} | |
| 7 | pZE-TH2 | From pZE12, dual operons, P_{L}lacO1; | 21 |
| | | RgTAL and EcHpaBC | |
| 8 | pCA1 | pTrcHis2B carrying codon-optimized | 23 |
| | | R. glutinis TAL | |
| 9 | pCA3 | pCDFDuet-1 carrying codon- | 23 |
| | | optimized R. glutinis TAL with a trc | |
| | | promoter | |
| 10 | pETM6-RgTAL^{syn} | #1 with RgTALsyn | This Study |
| 11 | pETM6-RgTALsyn- | #1 with RgTALsyn, HpaB, and HpaC | This Study |
| | HpaBC | in monocistronic form | |
| 12 | pETM6-HpaBC | #1 with HpaB and HpaC in | 17 |
| | | monocistronic form | |
| 13 | pXylA | #1 with constitutive PxylA promoter | This Study |
| 14 | pXPA-fapO-eGFP | pGAP promoter, rrnB terminator and | 33 |
| | | ePathBrick feature carrying one copy | |
| | | of fapO and eGFP | |
| 15 | pXylA-RgTAL^{syn} | #13 carrying RgTAL^{syn} | This Study |
| 16 | pXPA-fapO- | #14 carrying RgTAL^{syn} | This Study |
| | RgTAL^{syn} | | |
| 17 | pMM1522 | Amp^{R} (E. coli), Tet^{R} (B. meg), | Mobitec |
| | | pBR322 ori, P_{xylA} | |
| 18 | pETM6-At3GT | #1 with 3GT from A. thaliana | This Study |
| 19 | pETM6-PhANS | #1 with ANS from P. hybrida | This Study |
| 20 | pETM6-At3GT- | #1 with At3GT and PhANS, | This Study |
| | PhANS | monocistronic | |

TABLE 4
Primers used in this study.

| Primer ID | Primer Name | Sequence (5′→3′) |
|---|---|---|
| 1 | ANS_XbaI_F | CCCTCTAGAAATAATTTTGTTT |
| (SEQ ID NO: 7) | | AACTTTAAGAAGGAGATATAC |
| | | ATATGGTGAATGCAGTAGTTA |
| | | C |
| 2 | ANS_XhoI_R | CGATCTCGAGCTATTTAGATTC |
| (SBQ ID NO: 8) | | TTCAGCAGCAAC |
| 3 | 3GT_NdeI_F | GCATCATATGACCAAACCCTC |
| (SEQ ID NO: 9) | | CGACC |
| 4 | 3GT_XhoI_R | CGATCTCGAGTCAAATAATGT |
| (SEQ ID NO: 10) | | TTACAACTGCATCC |
| 5 | pETM6_ALL_inserts_flank_F | CCATCGGTGATGTCGGCGATA |
| (SEQ ID NO: 11) | | TAGG |
| 6 | pETM6_ALL_inserts_flank_R | GTCGAGGTGCCGTAAAGCACT |
| (SEQ ID NO: 12) | | AAATCG |
| 7 | ANS_mid_seq_F | CCATCTGGCCTAAAAATCCTA |
| (SEQ ID NO: 13) | | CTGACTACAC |
| 8 | ANS_mid_seq_R | CCTCTTTGAAGACTTTGTGTTC |
| (SEQ ID NO: 14) | | AACAGCG |
| 9 | 3GT_mid_seq_F | GCTTCATCAAATGGGTCTTGCT |
| (SEQ ID NO: 15) | | TTGC |
| 10 | 3GT_mid_seq_R | GGTGTCATGACCGTACCAAAG |
| (SEQ ID NO: 16) | | CTAATG |
| 11 | RgTALsyn_FWD w/NdeI | GCGGCGCATATGGCGCCTCGC |
| (SEQ ID NO: 17) | | CCGACTTC |
| 12 | RgTALsyn_REV w/SpeI | GCGGCGACTAGTTTATGCCAG |
| (SEQ ID NO: 18) | | CATCTTCAGCAGAACATTG |
| 13 | SDM_RgTALsyn_FWD | GCACTGCACGACGCGCACATG |
| (SEQ ID NO: 19) | | TTGAGCCTGTTGAGC |
| 14 | SDM_RgTALsyn_REV | GCTCAACAGGCTCAACATGTG |
| (SEQ ID NO: 20) | | CGCGTCGTGCAGTGC |
| 15 | pXylA_FOR | GCAAGCATGCGAAATGCA |
| (SEQ ID NO: 21) | | |
| 16 | pXylA_REV | GAGTTTCGTTCGAGATCGC |
| (SEQ ID NO: 22) | | |

TABLE 5
gBlock Sequence for cloning pXy1A (SEQ ID NO: 23)

GCAAGCATGCGAAATGCACCTAGGAAAAAAAACATTGAAATAAACATTTA
TTTTGTATATGATGAGATAAAGTTAGTTTATTGGATAAACAAACTAACTC
AATTAAGATAGTTGATGGATAAACTTGTTCACTTAAATCAAAGGGGGAAA
TGTACACATATGGCAGATCTCAATTGGATATCGGCCGGCCACGCGATCGC
TGACGTCGGTACCCTCGAGTCTGGTAAAGAAACCGCTGCTGCGAAATTTG
AACGCCAGCACATGGACTCGTCTACTAGTCGCAGCTTAATTAAGCGATCT
CGAACGAAACTC

Petunia.times.hybrida anthocyanidin synthase (PhANS) was amplified with primers 1 and 2 using plasmid pMAL-PhANS (unpublished) as a template, and Arabidopsis thaliana anthocyanidin 3-O-glucosyltransferase (At3GT) was amplified with primers 3 and 4 using plasmid pMAL-At3GT (unpublished) as a template. Following restriction digestion of PCR amplicon PhANS (XbaI/XhoI), PCR amplicon At3GT (NdeI/XhoI), and vector pETM6 (XbaI/XhoI for PhANS and NdeI/XhoI for At3GT), digested products were gel purified and ligated (Rapid DNA Ligation Kit, Thermo Scientific) to construct plasmids pETM6-PhANS and pETM6-At3GT. Constructs were transformed into DH5α and confirmed by Sanger sequencing with primers 5-10. Using the ePathBrick sub-cloning procedure, At3GT and PhANS were then assembled into monocistronic configuration by ligation of restriction digestion fragments from plasmid pETM6-At3GT (NheI/SalI) and pETM6-PhANS (AvrII/SalI), yielding plasmid pETM6-At3GT-m-PhANS.

### EXAMPLE 17

### TAL Module, C5 Module, p168 Module, and Antho Module Polyculture—Fermentation Protocol

The small scale cultivation protocol was adapted from with only minor modification. Except where noted, the cultures were grown in AMM with 20 g/L glucose as the primary carbon source. The cultures were first grown at 37° C. and transitioned to 30° C. upon induction with 1 mM IPTG. In the case of the phenylpropanoic acid production strains, 125 mL non-baffled shake flasks containing 25 mL of media were used to confirm small scale screening studies, allow for more frequent sampling, and limit evaporation effects on final titer.

### EXAMPLE 18

### TAL Module, C5 Module, p168 Module, and Antho Module Polyculture—Metabolite Analysis

A 25 μL injection was used for all polyculture fermentations. Analysis of phenylpropanoic acid titers in monoculture required a 10-fold dilution of culture broth and a 5 μL injection volume to reach the linear region for UV detection. Absorbance at 280 nm was monitored in all cases except for anthocyanidin-3-glucosides where 518 nm was used. Product titers were determined using authentic standards, while (+)-afzelechin was quantified using the (+)-catechin standard curve in accordance with previous literature, because (+)-afzelechin is not commercially available. All experiments were performed in at least biological duplicate, with key high-titer conditions reproduced in biological and experimental triplicate. Error bars represent +1 standard deviation from the mean. Significance of data was determined using a two-tailed unpaired t-test with a 95 percent confidence interval.

### EXAMPLE 19

### TAL Module, C5 Module, p168 Module, and Antho Module Polyculture—Results

Expanding upon previous co-culture efforts, the development of two additional bioconversion modules has been accomplished to realize the de novo production of both flavan-3-ols and anthocyanidin-3-glucosides for the first time outside of plants. FIG. 5 shows polyculture schematic representing the realized 4-strain polyculture. Inclusion of fifth strain shows potential for extension through addition of sequential modules.

### EXAMPLE 20

### TAL Module, C5 Module, p168 Module, and Antho Module Polyculture—Development of TAL Module

Significant efforts have been focused on improving the de novo production of phenylpropanoic acids in E. coli. Efforts from both the Stephanopoulos and Yan labs have enabled the near gram-scale production of both p-coumaric and caffeic acid. The development of the tyrosine overproducing E. coli strain rpoA14(DE3) represents a major milestone for the de novo production of phenylpropanoic acids, while the discovery and optimization of the native E. coli non-P450 hydroxylase enabled, for the first time, efficient production of caffeic acid through the ortho-hydroxylation of p-coumaric acid. Building off of these efforts, we set out to develop a phenylpropanoic acid production module that was compatible with our previously described ‘C5’ and ‘p168’ modules to enable the de novo production of flavan-3-ols in vivo.

TABLE 6
Twenty-eight potential phenylpropanoic acid production
modules. ‘Q’ in the strain name indicates strain QH4,
while ‘R’ in strain name indicates strain rpoA14(DE3)

| Name | Plasmids |
|---|---|
| Q1 | pZE-TH2, pCS-TPTA |
| Q2 | pZE-TH2 |
| Q3 | pETM6-RgTAL^{syn}, pCS-TPTA |
| Q4 | pETM6-RgTAL^{syn} |
| Q5 | pCA1, pCS-TPTA |
| Q6 | pCA1 |
| Q7 | pCA3, pCS-TPTA |
| Q8 | pCA3 |
| Q9 | pETM6-RgTAL^{syn}-HpaBC, pCS-TPTA |
| Q10 | pETM6-RgTAL^{syn}-HpaBC |
| Q11 | pXPA-RgTAL^{syn} |
| Q12 | pXPA-RgTAL^{syn}, pCS-TPTA |
| Q13 | pXylA-RgTAL^{syn} |
| Q14 | pXylA-RgTAL^{syn}, pCS-TPTA |
| R1 | pZE-TH2, pCS-TPTA |
| R2 | pZE-TH2 |
| R3 | pETM6-RgTAL^{syn}, pCS-TPTA |
| R4 | pETM6-RgTAL^{syn} |
| R5 | pCA1, pCS-TPTA |
| R6 | pCA1 |
| R7 | pCA3, pCS-TPTA |
| R8 | pCA3 |
| R9 | pETM6-RgTAL^{syn}-HpaBC, pCS-TPTA |
| R10 | pETM6-RgTAL^{syn}-HpaBC |
| R11 | pXPA-RgTAL^{syn} |
| R12 | pXPA-RgTAL^{syn}, pCS-TPTA |
| R13 | pXylA-RgTAL^{syn} |
| R14 | pXylA-RgTAL^{syn}, pCS-TPTA |

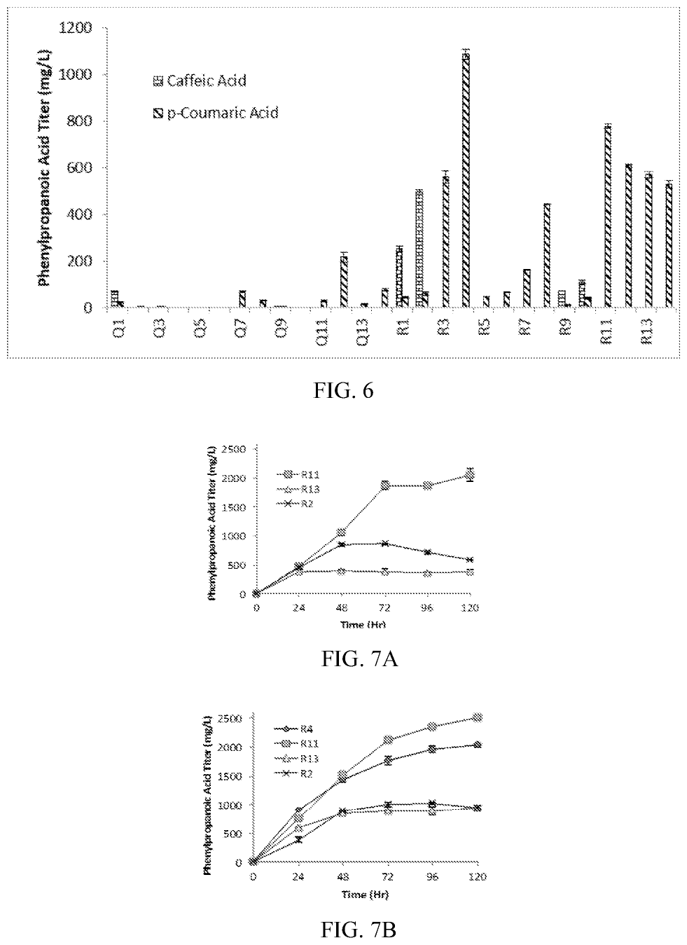

To accomplish this task, we collected the most efficient plasmids and strains from the recent literature and along with several plasmids constructed in the Koffas' lab, built 28 strain-plasmid combinations for screening of phenylpropanoic acid production, Table 6. Twenty of the 28 strains were designed for p-coumaric acid production (TAL overexpression), while the remaining 8 were targeted for caffeic acid production (TAL and HpaBC overexpression). The effect of the endogenous gene supplementation plasmid, pCS-TPTA, was also tested but did not show significant titer improvements for any of the tested combinations, FIG. 6. From the strain combinations, strain R4 represented the best p-coumaric acid production, while strain R2 was selected as the best caffeic acid producer. It is interesting to note that neither R2 nor R4 represent a strain configuration that had been previously published indicating that significant improvements can be realized through basic literature review and combinatorial screening of available modules.

### EXAMPLE 21

### TAL Module, C5 Module, p168 Module, and Antho Module Polyculture—Optimization of Phenylpropanoic Acid Production

Three p-coumaric acid (R4, R11, R13) and one caffeic acid (R2) production strains from the initial screen were subjected to further optimization to determine the full potential of these modules in monoculture. Through course optimization of induction point, inducer concentration, production temperature, and carbon source, the highest titer production to date was realized for both p-coumaric and caffeic acid at 2.51±0.03 and 1.03±0.02 g/L, respectively (FIG. 7). The production of p-coumaric acid was found to be highly sensitive to nearly all optimization parameters with highest titer production occurring in glycerol-based media (FIG. 7). Interestingly, caffeic acid production with strain R2 was found to be relatively insensitive to all factors. The titers presented here represent a 258% and 134% improvement for p-coumaric and caffeic acid, respectively, over the highest titers reported in the literature to date. Future efforts to scale-up to fed batch fermentation are underway to further improve phenylpropanoic acid titers, yields, and productivity.

### EXAMPLE 22

### TAL Module, C5 Module, p168 Module, and Antho Module Polyculture—Production of Flavan-3-Ols De Novo

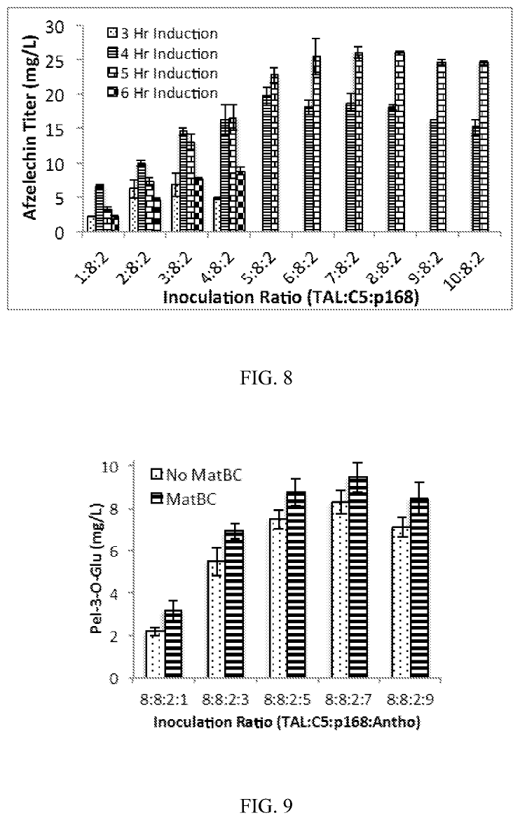

Combining the previously published co-culture system for the efficient production of flavan-3-ols from phenylpropanoic acids with the recently developed phenylpropanoic acid production module enables the production of flavan-3-ols from glucose. Highlighting the drop-in modularity of polyculture systems we conserved the previously optimized ratio of C5:p168 of 8:2 and varied only the proportion of the TAL module over several induction points in the range of the predicted optimum from previous work. Using this simple optimization strategy, we were able to demonstrate the de novo production of afzelechin for the first time in a microbial host (FIG. 8). Furthermore, we were also to demonstrate production titers of 26.1±0.8 mg/L without extensive optimization. These successes supported the further expansion of flavonoid production using the polyculture platform.

### EXAMPLE 23

### TAL Module, C5 Module, p168 Module, and Antho Module Polyculture—Production of Anthocyanidin-3-Glucosides De Novo

Our previous successes using polycultures for the production of flavonoids has urged the further application of this technology to expand what is currently possible in vivo. Previous efforts in the Koffas' lab have developed strains capable of high titer anthocyanidin-3-glucoside production from flavan-3-ols, but efforts to further extend the pathway towards the phenylpropanoic acid precursors have not been successful. Building off of these efforts, we cloned the previously characterized ANS and 3GT enzymes into a synthetic monocistronic operon in the ePathBrick plasmid pETM6. Transforming this plasmid into our baseline host BL21star™(DE3) resulted in our ‘Antho’ module to be combined with the previously described TAL, C5, and p168 modules for the de novo production of anthocyanidin-3-glucosides in vivo. In a similar fashion as before, the previously determined optimum ratio 8:8:2 (TAL:C5:p168) was conserved with the fraction of the new module being varied to result in the first account of a functional synthetic four strain polyculture. This microbial consortium enabled, for the first time outside of plants, the production of the anthocyanidin-3-glucoside, callistephin, from glucose, FIG. 9).

Adding two additional enzyme overexpressions, matBC, to the previously published C5 module, further highlights the flexibility of the polyculture platform for rapid expansion and modification. These enzymes enable the uptake of externally supplemented sodium malonate and subsequent activation to malonyl-CoA, a key and limiting substrate for the chalcone synthase enzyme. Significantly (p-value<0.05) higher production of callistephin from glucose was achieved across a wide range of inoculation ratios, while conserving the optimum fermentation conditions from previous experiments.

In summary, the rapid success of these polycultures to realize the de novo production of various late-pathway flavonoid metabolites demonstrates the power of these techniques over traditional monoculture metabolic engineering efforts. Additionally, the ease at which these pathways were re-optimized through conservation of the previously optimized inoculation ratio further highlights the benefits of polyculture modularity over that of traditional monoculture techniques. In traditional monoculture techniques, extending the current heterologous overexpression pathway would require additional genes to be cloned and expressed in the previously optimized strain, consequently un-optimizing the strain from both a genetic and fermentation perspective. Genetic re-optimization is a difficult task. Oftentimes, it is impossible to regain the fluxes previously achieved, due to increased metabolic burden or natural precursor and co-factor requirements, limiting the overall titer, yield and productivity of the process. Polycultures, however, enable the genetic optimization of each module to be conserved only requiring minor fermentation optimization to adjust the inoculation ratio of the new strain. The simplicity of this optimization and the smooth trends observed in corresponding production landscapes support the hypothesis that these cultures are stable through the production phase of the fermentation.

In conclusion, we have demonstrated the development of a high-titer phenylpropanoic acid module and a plan to demonstrate its true potential through bioreactor scale-up. Utilizing this module along with the previously published modules (C5 and p168), we demonstrate the de novo production of flavan-3-ols for the first time outside of the native plant hosts. Further expanding on this polyculture theme, we incorporated a fourth module (Antho) containing the genes ANS and 3GT. Using all four modules, we were able to demonstrate the production of the anthocyindin-3-glucoside, callistephin, from glucose. This feat was possible due to the modularity of the polyculture scaffold conserving the genetic optimization of each module only requiring basic fermentation optimization to achieve peak production. Finally, we outline the path forward for expanding upon this polyculture work. These plans include potential additional modules, expansion into the terpenoid and alkaloid pathways, and methods to address the stability of the individual strain populations with time. In summary, co-culture and polyculture techniques have demonstrated their potential to rapidly expand what is deemed to be possible with metabolic engineering, but this power comes with additional complexities that must be addressed from a systematic approach to achieve the highest titer, yield, and productivities possible.

### EXAMPLE 24

Sequences RtMatB-Rhizobium trifolii
Nucleic acid sequence
(SEQ ID NO: 24)
GTGAGCAACCATCTTTTCGACGCCATGCGGGCCGCCGCGCCCGGTAACGCACCATTC
ATCCGGATCGATAACACGCGCACATGGACCTATGACGACGCCGTCGCTCTTTCCGGC
CGCATTGCCGGCGCGATGGACACGCTCGGCATTCGCCCCGGCGACCGCGTTGCGGT
GCAGGTCGAGAAAAGTGCCGAGGCATTGATCCTCTATCTCGCCTGTCTTCGAAGCGG
CGCCGTTTACCTGCCGCTCAACACCGCCTATACGCTGGCTGAGCTCGATTATTTTATC
GGCGATGCGGAGCCGCGTTTGGTGGTTGTTGCATCGTCGGCTCGAGCGGGCGTGGA
GACAATCGCCAAGCCCCGCGGTGCGATCGTCGAAACTCTCGACGCTGATGGCAGCG
GCTCGTTGCTGGATCTCGCCCGCGATGAGCCGGCTGACTTTGTCGATGCCTCGCGCT
CCGCCGATGATCTGGCTGCGATCCTCTACACCTCGGGAACGACGGGACGCTCCAAG
GGGGCGATGCTCACGCATGGGAACCTGCTCTCGAACGCCCTGACCTTGCGAGATTTT
TGGCGCGTCACCGCCGGCGATCGACTGATCCATGCCTTGCCGATCTTCCACACGCAT
GGGCTGTTCGTCGCCACGAACGTCACTTTACTCGCCGGCGCCTCGATGTTCCTGCTG
TCGAAGTTCGACCCGGAGGAGATCCTGTCGCTGATGCCGCAGGCAACGATGCTGAT
GGGCGTGCCGACCTTCTACGTGCGCCTCCTGCAAAGCCCGCGCCTCGACAAGCAAG
CAGTCGCCAACATCCGCCTCTTCATTTCCGGTTCGGCTCCACTGCTTGCAGAAACAC
ATACCGAGTTCCAGGCACGTACCGGTCACGCCATTCTCGAGCGCTACGGCATGACG
GAAACCAATATGAACACGTCCAACCCTTATGAGGGGAAACGGATTGCCGGAACGGT
CGGCTTCCCGCTGCCTGATGTGACGGTGCGCGTCACCGATCCCGCCACCGGGCTCGC
GCTGCCGCCTGAAGAAACAGGCATGATCGAGATCAAGGGGCCGAACGTTTTCAAGG
GCTATTGGCGCATGCCCGAAAAAACCGCGGCCGAATTCACCGCCGACGGTTTCTTCA
TCAGCGGCGATCTCGGCAAGATCGACCGGGACGGTTATGTCCACATCGTCGGCCGT
GGCAAGGATCTGGTGATTTCCGGTGGATACAACATCTATCCGAAAGAGGTGGAGGG
CGAGATCGACCAGATCGAGGGTGTGGTTGAGAGCGCTGTGATCGGCGTGCCGCATC
CCGATTTCGGAGAAGGCGTGACCGCCGTCGTCGTGCGCAAACCCGGCGCTGTCCTCG
ATGAAAAGGCCATCGTCAGCGCCCTCCAGGACCGGCTCGCGCGCTACAAACAACCC
AAGCGCATCATCTTTGCCGAAGACTTGCCGCGCAACACGATGGGCAAGGTTCAGAA
AAACATCCTGCGGCAGCAATACGCCGATCTTTACACCAGGACGTAA
RtMatB-Rhizobium trifolii
Amino acid sequence
(SEQ ID NO: 25)
MSNHLFDAMRAAAPGNAPFIRIDNTRTWTYDDAVALSGRIAGAMDTLGIRPGDRVAVQ
VEKSAEALILYLACLRSGAVYLPLNTAYTLAELDYFIGDAEPRLVVVASSARAGVETIAK
PRGAIVETLDADGSGSLLDLARDEPADFVDASRSADDLAAILYTSGTTGRSKGAMLTHG
NLLSNALTLRDFWRVTAGDRLIHALPIFHTHGLFVATNVTLLAGASMFLLSKFDPEEILS
LMPQATMLMGVPTFYVRLLQSPRLDKQAVANIRLFISGSAPLLAETHTEFQARTGHAILE
RYGMTETNMNTSNPYEGKRIAGTVGFPLPDVTVRVTDPATGLALPPEETGMIEIKGPNV
FKGYWRMPEKTAAEFTADGFFISGDLGKIDRDGYVHIVGRGKDLVISGGYNIYPKEVEG
EIDQIEGVVESAVIGVPHPDFGEGVTAVVVRKPGAVLDEKAIVSALQDRLARYKQPKRII
FAEDLPRNTMGKVQKNILRQQYADLYTRT
RtMatC-Rhizobium trifolii
Nucleic acid sequence
(SEQ ID NO: 26)
ATGGGCATCGAACTGCTGAGTATTGGTCTGCTGATTGCTATGTTTATTATTGCTACGA
TTCAACCGATTAACATGGGTGCTCTGGCATTCGCAGGCGCTTTTGTGCTGGGTAGCA
TGATTATCGGCATGAAAACCAACGAAATTTTCGCAGGCTTTCCGTCTGACCTGTTTCT
GACCCTGGTGGCGGTTACGTACCTGTTTGCGATTGCCCAGATCAATGGCACCATCGA
CTGGCTGGTTGAATGCGCGGTGCGTCTGGTTCGTGGCCGCATTGGTCTGATCCCGTG
GGTGATGTTCCTGGTTGCGGCCATTATCACCGGTTTTGGTGCACTGGGTCCGGCAGC
TGTTGCAATTCTGGCACCGGTCGCACTGAGCTTCGCAGTGCAATATCGCATTCATCC
GGTTATGATGGGTCTGATGGTCATCCACGGCGCACAGGCTGGCGGTTTTTCACCGAT
TTCGATCTACGGCGGTATTACCAACCAAATCGTGGCAAAAGCAGGTCTGCCGTTCGC
ACCGACGAGTCTGTTTCTGAGCAGCTTTTTCTTTAATCTGGCAATTGCTGTCCTGGTG
TTCTTTGTGTTTGGCGGTGCACGTGTTATGAAACACGATCCGGCTTCTCTGGGTCCGC
TGCCGGAACTGCATCCGGAAGGCGTGAGCGCGTCTATTCGTGGTCATGGCGGCACC
CCGGCAAAACCGATCCGCGAACATGCGTATGGCACCGCAGCAGACACGGCAACCAC
GCTGCGTCTGAACAATGAACGCATTACCACGCTGATCGGTCTGACCGCACTGGGTAT
TGGTGCACTGGTTTTCAAATTTAACGTCGGTCTGGTGGCTATGACCGTGGCAGTGGT
TCTGGCACTGCTGAGCCCGAAAACGCAGAAAGCAGCTATTGATAAAGTCAGTTGGT
CCACCGTGCTGCTGATCGCGGGTATTATCACGTATGTTGGCGTCATGGAAAAAGCGG
GCACCGTTGACTACGTCGCCAATGGTATTAGTTCCCTGGGTATGCCGCTGCTGGTCG
CGCTGCTGCTGTGTTTCACCGGCGCCATCGTGTCCGCGTTTGCCTCATCGACGGCACT
GCTGGGTGCTATTATCCCGCTGGCCGTTCCGTTCCTGCTGCAGGGCCATATTAGTGC
AATCGGTGTCGTGGCGGCCATTGCTATCTCCACCACGATTGTGGATACCAGCCCGTT
TTCTACGAACGGCGCGCTGGTTGTCGCAAATGCTCCGGATGACTCACGTGAACAGGT
TCTGCGCCAACTGCTGATCTATTCGGCCCTGATTGCTATTATTGGTCCGATTGTCGCC
TGGCTGGTTTTCGTTGTGCCGGGTCTGGTCTAA
RtMatC-Rhizobium trifolii
Amino acid sequence
(SEQ ID NO: 27)
MGIELLSIGLLIAMFIIATIQPINMGALAFAGAFVLGSMIIGMKTNEIFAGFPSDLFLTLVA
VTYLFAIAQINGTIDWLVECAVRLVRGRIGLIPWVMFLVAAIITGFGALGPAAVAILAPV
ALSFAVQYRIHPVMMGLMVIHGAQAGGFSPISIYGGITNQIVAKAGLPFAPTSLFLSSFFF
NLAIAVLVFFVFGGARVMKHDPASLGPLPELHPEGVSASIRGHGGTPAKPIREHAYGTA
ADTATTLRLNNERITTLIGLTALGIGALVFKFNVGLVAMTVAVVLALLSPKTQKAAIDK
VSWSTVLLIAGIITYVGVMEKAGTVDYVANGISSLGMPLLVALLLCFTGAIVSAFASSTA
LLGAIIPLAVPFLLQGHISAIGVVAAIAISTTIVDTSPFSTNGALVVANAPDDSREQVLRQL
LIYSALIAIIGPIVAWLVFVVPGLV
RgTALsyn-Rhodotorula glutinis
Nucleic acid sequence
(SEQ ID NO: 28)
atggcgcctcgcccgacttcgcaaagccaggcccgcacttgcccgacgacgcaggttacccaagttgatatcgttgag
aaaatgttggcggctcctactgatagcacgctggagctggacggttatagcctgaatctgggtgatgtcgtgagcgct
gcgcgtaagggtcgtcctgtccgtgtcaaagatagcgatgaaatccgcagcaaaatcgacaagagcgttgaattcctg
cgcagccaactgagcatgtcggtttacggtgtgacgaccggattggcggctccgcggacacgcgcacggaggacgcaa
ttagcctgcaaaaggcgttgctggaacaccagctgtgtggtgtgttgccgagcagatcgacagattcgcttgggtcgt
ggtctggagaatagcctgccgttggaagtcgttcgcggtgcaatgaccattcgtgtgaattcgctgacccgtggccat
agcgctgttcgtctggttgttctggaagcactgacgaactttctgaaccacggtattaccccgattgttccgctgcgc
ggtacgatctccgcgagcggcgatctgtctccactgtcgtacattgcagcggcgattagcggtcacccggatagcaaa
gttcacgtggtccatgaaggcaaagagaagatcctgtacgcgcgcgaagcgatggcgctgtttaacctggagccggtg
gttttgggtccgaaggagggcctgggtctggtgaatggtacggcagtctccgcgagcatggcaacgctggcactgcac
gacgcgcatatgttgagcctgttgagccaatcgctgaccgcgatgaccgtggaggcgatggtcggtcacgcgggcaga
tccatccattcctgcacgatgttacgcgtccgcacccgacgcaaatcgaggtcgcgggtaacattcgcaaactgctgg
agggctcgcgcttcgcggtccaccacgaggaagaggttaaggtcaaggatgatgaaggcattttgcgtcaggatcgtt
atccgttgcgcacgagcccgcaatggttgggtccgctggtgtccgacctgattcacgctcatgccgtcttgacgatcg
aagcgggtcaaagcaccaccgataacccactgatcgatgttgagaataagaccagccatcacggtggcaactttcaag
cggcagcggttgccaacacgatggaaaagacccgtctgggcttggcccaaatcggtaaactgaatttcacccagctga
cggagatgctgaacgcgggcatgaatcgtggcttgccgagctgcctggcggctgaagacccatccctgagctatcatt
gcaaaggtctggacattgcggcggctgcatatacgagcgaactgggccacctggctaacccggtcaccacccacgtcc
aaccggctgaaatggcaaaccaggcggtgaatagcttggcgttgattagcgcacgtcgtaccacggaatctaacgacg
ttctgtccctgctgctggcaacgcacctgtactgcgtgctgcaggcgatcgacctgcgtgcgattgagttcgagttca
agaaacagtttggtcctgccattgttagcctgatcgaccaacactttggtagcgcgatgacgggtagcaatctgcgtg
atgagctggttgaaaaggtcaataagactctggccaagcgtttggagcaaaccaatagctacgatctggttccgcgct
ggcacgacgcttttagcttcgctgcaggcactgttgtcgaggttctgtccagcacgagcctgagcttggcggccgtga
acgcatggaaggttgcggcagccgagagcgcgatctccttgacgcgccaggtccgtgaaacgttttggtccgctgcaa
gcacctccagcccggcgttgtcttacttgagcccgcgcacgcagatcctgtacgcatttgtgcgtgaggaactgggtg
tcaaagcccgccgtggtgacgtcttcttgggtaaacaagaagttaccatcggcagcaacgttagcaagatttacgaag
ccatcaagagcggccgtatcaacaatgttctgctgaagatgctggcataa
RgTALsyn-Rhodotorula glutinis
Amino acid sequence
(SEQ ID NO: 29)
MAPRPTSQSQARTCPTTQVTQVDIVEKMLAAPTDSTLELDGYSLNLGDVVSAARKGRP
VRVKDSDEIRSKIDKSVEFLRSQLSMSVYGVTTGFGGSADTRTEDAISLQKALLEHQLCG
VLPSSFDSFRLGRGLENSLPLEVVRGAMTIRVNSLTRGHSAVRLVVLEALTNFLNHGITPI
VPLRGTISASGDLSPLSYIAAAISGHPDSKVHVVHEGKEKILYAREAMALFNLEPVVLGP
KEGLGLVNGTAVSASMATLALHDAHMLSLLSQSLTAMTVEAMVGHAGSFHPFLHDVT
RPHPTQIEVAGNIRKLLEGSRFAVHHEEEVKVKDDEGILRQDRYPLRTSPQWLGPLVSDL
IHAHAVLTIEAGQSTTDNPLIDVENKTSHEIGGNFQAAAVANTMEKTRLGLAQIGKLNFT
QLTEMLNAGMNRGLPSCLAAEDPSLSYHCKGLDIAAAAYTSELGHLANPVTTHVQPAE
MANQAVNSLALISARRTTESNDVLSLLLATHLYCVLQAIDLRAIEFEFKKQFGPAIVSLID
QHFGSAMTGSNLRDELVEKVNKTLAKRLEQTNSYDLVPRWHDAFSFAAGTVVEVLSST
SLSLAAVNAWKVAAAESAISLTRQVRETFWSAASTSSPALSYLSPRTQILYAFVREELGV
KARRGDVFLGKQEVTIGSNVSKIYEAIKSGRINNVLLKMLA
At4CL-Arabidopsis thaliana
Nucleic acid sequence
(SEQ ID NO: 30)
atggcgccacaagaacaagcagtttctcaggtgatggagaaacagagcaacaacaacaacagtgacgtcattttccgatcaaagttaccgg
atatttacatcccgaaccacctatctctccacgactacatcttccaaaacatctccgaattcgccactaagccttgcctaatcaacggaccaacc
ggccacgtgtacacttactccgacgtccacgtcatctcccgccaaatcgccgccaattttcacaaactcggcgttaaccaaaacgacgtcgt
catgctcctcctcccaaactgtcccgaattcgtcctctctttcctcgccgcctccttccgcggcgcaaccgccaccgccgcaaaccctttcttc
actccggcggagatagctaaacaagccaaagcctccaacaccaaactcataatcaccgaagctcgttacgtcgacaaaatcaaaccacttc
aaaacgacgacggagtagtcatcgtctgcatcgacgacaacgaatccgtgccaatccctgaaggctgcctccgcttcaccgagttgactca
gtcgacaaccgaggcatcagaagtcatcgactcggtggagatttcaccggacgacgtggtggcactaccttactcctctggcacgacggg
attaccaaaaggagtgatgctgactcacaagggactagtcacgagcgttgctcagcaagtcgacggcgagaacccgaatctttatttccaca
gcgatgacgtcatactctgtgttttgcccatgtttcatatctacgctttgaactcgatcatgttgtgtggtcttagagttggtgcggcgattctgata
atgccgaagtttgagatcaatctgctattggagctgatccagaggtgtaaagtgacggtggctccgatggttccgccgattgtgttggccattg
cgaagtatcggagacggagaagtatgatttgagctcgataagagtggtgaaatctggtgctgctcctcttggtaaagaacttgaagatgccg
ttaatgccaagtttcctaatgccaaactcggtcagggatacggaatgacggaagcaggtccagtgctcgcaatgtcgttaggttttgcaaagg
aaccttttccggttaagtcaggagcttgtggtactgttgtaagaaatgctgagatgaaaatagttgatccagacaccggagattctctttcgagg
aatcaacccggtgagatttgtattcgtggtcaccagatcatgaaaggttacctcaacaatccggcagctacagcagagaccattgataaaga
cggttggcttcatactggagatattggattgatcgatgacgatgacgagcttttcatcgttgatcgattgaaagaacttatcaagtataaaggtttt
caggtagctccggctgagctagaggctttgctcatcggtcatcctgacattactgatgttgctgttgtcgcaatgaaagaagaagcagctggt
gaagttcctgttgcatttgtggtgaaatcgaaggattcggagttatcagaagatgatgtgaagcaattcgtgtcgaaacaggttgtgttttacaa
gagaatcaacaaagtgttcttcactgaatccattcctaaagctccatcagggaagatattgaggaaagatctgagggcaaaactagcaaatg
gattgtga
At4CL-Arabidopsis thaliana
Amino acid sequence
(SEQ ID NO: 31)
MAPQEQAVSQVMEKQSNNNNSDVIFRSKLPDIYIPNHLSLHDYIFQNISEFATKPCLINGP
TGHVYTYSDVHVISRQIAANFHKLGVNQNDVVMLLLPNCPEFVLSFLAASFRGATATAA
NPFFTPAEIAKQAKASNTKLIITEARYVDKIKPLQNDDGVVIVCIDDNESVPIPEGCLRFTE
LTQSTTEASEVIDSVEISPDDVVALPYSSGTTGLPKGVMLTHKGLVTSVAQQVDGENPNL
YFHSDDVILCVLPMFHIYALNSIMLCGLRVGAAILIMPKFEINLLLELIQRCKVTVAPMVP
PIVLAIAKSSETEKYDLSSIRVVKSGAAPLGKELEDAVNAKFPNAKLGQGYGMTEAGPV
LAMSLGFAKEPFPVKSGACGTVVRNAEMKIVDPDTGDSLSRNQPGEICIRGHQIMKGYL
NNPAATAETIDKDGWLHTGDIGLIDDDDELFIVDRLKELIKYKGFQVAPAELEALLIGHP
DITDVAVVAMKEEAAGEVPVAFVVKSKDSELSEDDVKQFVSKQVVFYKRINKVFFTESI
PKAPSGKILRKDLRAKLANGL
Pc4CL-Petroselinum crispum
Nucleic acid sequence
(SEQ ID NO: 32)
atgggagactgtgtagcacccaaagaagaccttattttccgatcgaaactccctgatatttacatcccgaaacaccttccgttacatacttattgt
ttcgaaaacatctcgaaagttggcgacaagtcctgtttaataaatggcgctacaggcgaaacgttcacttattcccaagttgagctcctttccag
gaaagttgcatcagggttaaacaaactcggcattcaacagggcgataccatcatgcttttgctccctaactcccctgagtattttttcgctttctta
ggcgcatcgtatcgtggtgcaatttctactatggccaatccgtttttcacttctgctgaggtgatcaaacagctcaaagcatcccaagctaagct
cataattacgcaagcttgttacgtagacaaagtgaaagactacgcagcagagaaaaatatacagatcatttgcatcgatgatgctcctcagga
ttgtttacatttctccaaacttatggaagctgatgaatcagaaatgcctgaggttgtgatcaattcagacgatgtcgtcgcgttaccttactcatcg
ggtactacaggactaccgaaaggtgttatgttgacacacaaaggacttgttactagcgtggcacaacaagttgatggagacaatccgaattta
tatatgcatagcgaggatgtgatgatctgcatattgcctttgtttcatatttattcgcttaacgcggtgttgtgctgtggactcagagcaggggtga
cgatcttgattatgcagaaatttgatattgtgccatttttggaactgatacagaaatataaagttacaattggaccgtttgtgccaccaattgtgttg
gcaattgcgaaaagtccagtggtggataaatatgacttgtcgtcggtgaggacggttatgtctggagctgctccgttagggaaggagcttga
agatgctgttagagctaagtttcctaatgccaaacttggtcagggatatggaatgacagaggcagggccagttttagcaatgtgcctggcgttt
gcaaaggaaccatacgagatcaaatcgggtgcctgtggaactgttgtgaggaatgctgaaatgaaaattgtggatcctgagaccaacgcct
ctcttccacgaaaccaacgcggagagatttgcattcgaggtgaccaaattatgaaaggctacctcaatgatcctgaatcaacaaggacaaca
atagacgaagaaggctggttgcacacaggagatataggcttcattgacgacgatgatgagctatttattgttgatagacttaaggaaataatca
aatacaaaggatccaggttgcccctgctgaacttgaagctctgctacttactcatcctaccatttccgatgctgcagttgttcccatgatagatg
agaaagcaggagaggtgcctgtggatttgttgtgagaacaaacggificaccaccactgaggaagaaatcaagcaattcgtctcgaaaca
ggtggtgttctacaagagaatatttcgtgtattttttgttgatgcaattccgaaatcaccatctggaaagattcttcgaaaggacttgagagcaaa
aatagcatccggtgatcttcccaaataa
Pc4CL-Petroselinum crispum
Amino acid sequence
(SEQ ID NO: 33)
MGDCVAPKEDLIFRSKLPDIYIPKHLPLHTYCFENISKVGDKSCLINGATGETFTYSQVEL
LSRKVASGLNKLGIQQGDTIMLLLPNSPEYFFAFLGASYRGAISTMANPFFTSAEVIKQLK
ASQAKLIITQACYVDKVKDYAAEKNIQIICIDDAPQDCLHFSKLMEADESEMPEVVINSD
DVVALPYSSGTTGLPKGVMLTHKGLVTSVAQQVDGDNPNLYMHSEDVMICILPLFHIYS
LNAVLCCGLRAGVTILIMQKFDIVPFLELIQKYKVTIGPFVPPIVLAIAKSPVVDKYDLSS
VRTVMSGAAPLGKELEDAVRAKFPNAKLGQGYGMTEAGPVLAMCLAFAKEPYEIKSG
ACGTVVRNAEMKIVDPETNASLPRNQRGEICIRGDQIMKGYLNDPESTRTTIDEEGWLH
TGDIGFIDDDDELFIVDRLKEIIKYKGFQVAPAELEALLLTHPTISDAAVVPMIDEKAGEV
PVAFVVRTNGFTTTEEEIKQFVSKQVVFYKRIFRVFFVDAIPKSPSGKILRKDLRARIASG
DLPK
Vv4CL-Vitis vinifera
Nucleic acid sequence
(SEQ ID NO: 34)
atgattagtattgaaacgcaaaacccggatgttagcaacctggacacctcgcactctattccgaaaatggcaaaccgtattgatgaccatgtgt
ttcgttctaaactgccggaaattccgatcagtaaccatctgccgctgcacacgtattgcttcgaaaattactcgcagtttgcagaccgtccgtgt
ctgattgttggctcgacgaacaaaacctatagatcgctgaaacccatctgatctctcgcaaagtgggcgcaggttttgctcacctgggtctga
aacagggcgatgtggttatgattctgctgcaaaattgcgcggaatttgccttcagctttctgggtgcgtctatggttggcgccgtcaccacgac
cgcaaacccgttctacacgtccgcggaaatcttcaaacagctgaacgcatcaaaagctaaaatcgtcgtgacccaggcgcaatatgtggat
aaactgcgcgactacccggatggtcaagttgccaaaattggcgaaggtttcacggtcattaccatcgatgacccgccggaaaactgtatgca
ttttagtgttgtctccgaagcgaacgaaagcgaactgccggaagtctcaattaattoggatgacccggtggccctgccgtttagctctggtac
gaccggcctgccgaaaggcgtggttctgacgcacaaatcactgatcacctcggtcgcccagcaagtggatggtgaaaacccgaatctgca
tctgaccccggatgacgtcgtgctgtgcgtgctgccgctgttccacatttatagcctgaactctgttctgctgtgtagtctgcgtgcaggtgcag
cagtgctgctgatgcagaaatttgaaattggtaccctgctggaactgatccaacgttaccgcgtgagcgttgcagctgttgtcccgccgctgg
ttctggcactggctaaaaatccgatggtggaatcgtttgatctgagttccatccgtgtggttctgagcggtgcagcaccgctgggcaaagaac
tggaagcagctctgcgttcccgcgttccgcaggcagtcctgggccaaggttatggcatgacggaagcaggcccggtgctgtcaatgtgcct
gggtttcgctaaacagccgtttccgacgaaatcaggttcgtgtggcaccgtcgtgcgtaacgcggaactgaaagttgtggatccggaaacc
ggttgctccctgggccgtaatcagccgggtgaaatttgtatccgcggccagcaaattatgaaaggttatctgaatgatccggaagcgacggc
ctctaccattgacgttgatggctggctgcataccggtgacatcggctacgtggatgacgatgaagaagtgttcattgttgatcgcgtcaaaga
actgatcaaattcaaaggtMcaggttccgccggcagaactggaagctctgctggtgtctcacccgtccattgccgatgcggccgtggttcc
gcaaaaagacgatgttgctggcgaagtcccggtggcgttcgtcgtgcgttctaacggttttgaactgaccgaagaagcagtgaaagaattca
tcagtaaacaggttgtcttttataaacgcctgcataaagtgtactttgttcacgcgattccgaaaagcccgtctggcaaaatcctgcgtaaagat
ctgcgcgcgaaactggccgaaaaaaccccggaaccgaac
Vv4CL-Vitis vinifera
Amino acid sequence
(SEQ ID NO: 35)
MISIETQNPDVSNLDTSHSIPKMANRIDDHVFRSKLPEIPISNHLPLHTYCFENYSQFADRP
CLIVGSTNKTYSFAETHLISRKVGAGFAHLGLKQGDVVMILLQNCAEFAFSFLGASMVG
AVTTTANPFYTSAEIFKQLNASKAKIVVTQAQYVDKLRDYPDGQVAKIGEGFTVITIDDP
PENCMHFSVVSEANESELPEVSINSDDPVALPFSSGTTGLPKGVVLTHKSLITSVAQQVD
GENPNLHLTPDDVVLCVLPLFHIYSLNSVLLCSLRAGAAVLLMQKFEIGTLLELIQRYRV
SVAAVVPPLVLALAKNPMVESFDLSSIRVVLSGAAPLGKELEAALRSRVPQAVLGQGYG
MTEAGPVLSMCLGFAKQPFPTKSGSCGTVVRNAELKVVDPETGCSLGRNQPGEICIRGQ
QIMKGYLNDPEATASTIDVDGWLHTGDIGYVDDDEEVFIVDRVKELIKFKGFQVPPAEL
EALLVSHPSIADAAVVPQKDDVAGEVPVAFVVRSNGFELTEEAVKEFISKQVVFYKRLH
KVYFVHAIPKSPSGKILRKDLRAKLAEKTPEPN
PhCHS-Petunia X hybrida
Nucleic acid sequence
(SEQ ID NO: 36)
atggtgacagtcgaggagtatcgtaaggcacaacgtgctgaaggtccagccactgtcatggccattggaacagccacaccttcaaactgtg
ttgatcaaagcacttaccctgatttttattttcgtatcactaacagtgagcacaagactgatcttaaggagaaatttaagcgcatgtgtgaaaaat
caatgattaagaaaaggtacatgcacttaacagaggaaatcttgaaagagaatcctagtatgtgtgaatacatggcaccttctcttgatgctag
gcaagacatagtggtggttgaagtgcccaaacttggcaaagaggcagctcaaaaggccatcaaggaatggggccagcccaagtccaaaa
ttacccatttggtcttttgcacaaccagtggtgtggacatgcctgggtgtgactatcaactcactaagctacttgggcttcgtccatcggtcaag
aggcttatgatgtaccaacaaggttgctttgctggtggcacggttcttcggttagccaaggacttggctgaaaacaacaagggcgctcgagt
ccttgttgtttgttcagaaatcaccgcggtcactttccgtgggccaaatgatactcatttggatagtttagttggccaagcactttttggtgatggg
gcaggcgcgatcattataggttctgatccaattccaggggtcgaaaggcctttgttcgagctcgtttcagcagcccaaactcttctcccagata
gccatggtgctattgatggccatctccgtgaagttgggcttacattccacttactcaaagatgttcctgggctgatctcaaaaaatattgagaag
agccttgaggaagcattcaaacctttgggcatttctgattggaactctctattctggattgctcatccaggtgggcctgcaattttggaccaagtt
gaaataaagttgggcctaaagcccgagaaacttaaggctacaaggaatgtgttaagtaactatggtaacatgtcaagtgcttgtgtactgtttat
tttggatgaaatgagaaaggcctcagccaaagaaggtttaggaactactggtgaagggcttgagtggggtgttctttttggatttgggcctgg
gctaacagttgagactgttgtcctccacagtgttgctacttaa
PhCHS-Petunia X hybrida
Amino acid sequence
(SEQ ID NO: 37)
MVTVEEYRKAQRAEGPATVMAIGTATPSNCVDQSTYPDFYFRITNSEHKTDLKEKFKR
MCEKSMIKKRYMHLTEEILKENPSMCEYMAPSLDARQDIVVVEVPKLGKEAAQKAIKE
WGQPKSKITHLVFCTTSGVDMPGCDYQLTKLLGLRPSVKRLMMYQQGCFAGGTVLRL
AKDLAENNKGARVLVVCSEITAVTFRGPNDTHLDSLVGQALFGDGAGAIIIGSDPIPGVE
RPLFELVSAAQTLLPDSHGAIDGHLREVGLTFHLLKDVPGLISKNIEKSLEEAFKPLGISD
WNSLFWIAHPGGPAILDQVEIKLGLKPEKLKATRNVLSNYGNMSSACVLFILDEMRKAS
AKEGLGTTGEGLEWGVLFGFGPGLTVETVVLHSVAT
CmCHS-Citrus maxima
Nucleic acid sequence
(SEQ ID NO: 38)
atggctacggtccaagaaatccgcaacgctcaacgcgcagatggtccggcgacggtcctggcaatcggcacggcaaccccggctcatag
cgtgaaccaggcagattatccggactattactttcgtattaccaaatctgaacacatgacggaactgaaagaaaaattcaaacgtatgtgcgat
aaaagtatgattaaaaaacgctacatgtacctgaccgaagaaatcctgaaagaaaacccgaatatgtgtgcctacatggcaccgagcctgg
atgcgcgccaggacattgtggttgtcgaagttccgaaactgggtaaagaagcggccaccaaagccatcaaagaatggggccaaccgaaa
tcaaaaattacgcacctgatcttttgcaccacgtcgggtgtggatatgccgggtgcagactatcagctgaccaaactgctgggtctgcgtccg
agcgttaaacgctttatgatgtaccagcaaggctgcttcgcaggcggtacggtcctgcgtctggctaaagatctggcggaaaacaataaag
gtgctcgcgttctggtggtttgtagtgaaattaccgctgtcacgtttcgtggtccggcggatacccatctggactccctggttggccaggccct
gttcggcgatggtgcagctgcggttatcgtcggcgcagatccggacacgagtgtggaacgtccgctgtatcagctggtttcaacctcgcaa
acgattctgccggattccgacggtgcgatcgatggccatctgcgcgaagtgggtctgacctttcacctgctgaaagacgttccgggcctgat
ttcaaaaaacatcgaaaaaagcctgtctgaagcctttgcaccggttggtatttcggattggagctctattttctggatcgcacatccgggcggtc
cggcaatcctggaccaggtggaaagcaaactgggtctgaaagaagaaaaactgaaagctacccgtcaagtcctgtctgaatacggcaata
tgagttccgcgtgtgtgctgttcattctggatgaaatgcgcaaaaaatctgccgaagaagctaaagcgaccacgggcgaaggtctggattgg
ggcgtgctgtttggtttcggtccgggtctgaccgtcgaaacggtcgtgctgcacagtgtgccgatcaaagcgggcggtggcggttccggcg
gtggtggtagtggtggtggtggctctccgccgccggccctgccgccgaaacgtcgtcgctaa
CmCHS-Citrus maxima
Amino acid sequence
(SEQ ID NO: 39)
MATVQEIRNAQRADGPATVLAIGTATPAHSVNQADYPDYYFRITKSEHMTELKEKFKR
MCDKSMIKKRYMYLTEEILKENPNMCAYMAPSLDARQDIVVVEVPKLGKEAATKAIKE
WGQPKSKITHLIFCTTSGVDMPGADYQLTKLLGLRPSVKRFMMYQQGCFAGGTVLRLA
KDLAENNKGARVLVVCSEITAVTFRGPADTHLDSLVGQALFGDGAAAVIVGADPDTSV
ERPLYQLVSTSQTILPDSDGAIDGHLREVGLTFHLLKDVPGLISKNIEKSLSEAFAPVGISD
WSSIFWIAHPGGPAILDQVESKLGLKEEKLKATRQVLSEYGNMSSACVLFILDEMRKKS
AEEAKATTGEGLDWGVLFGFGPGLTVETVVLHSVPIKAGGGGSGGGGSGGGGSPPPAL
PPKRRR
CmCHI-Citrus maxima
Nucleic acid sequence
(SEQ ID NO: 40)
atgaatccgtcgccgtctgttaccgaactgcaagtggaaaatgtcacctttacgccgagtctgcaaccgccgggctctaccaaatcgcatttt
ctgggcggtgcaggtgaacgtggcctggaaatcgaaggcaaatttgttaaattcaccgctattggtgtctatctggaagaaaacgccgtgcc
gctgctggcaggcaaatggaaaggcaaaaccgccggtgaactgacggaatctgtcgaatttttccgcgatgtggttaccggcccgtttgaa
aaattcatgaaagtgaccatgatcctgccgctgacgggtgcgcagtattcagaaaaagttgctgaaaattgcatggcgatttggaaatttttcg
gcatctacaccgatgcagaagctaaagcgattgaaaaatttacggaagtgttcaaagacgaaatttttccgccgggcagctctatcctgttca
cccaaagttccggttcgctgacgatttcattttcgaaagatggcagcatcccgaaagacggtgtcgcggtgattgaaaacaatctgctgagc
gaagccgttctggaatctatgatcggtaaaaacggcgtcagtccggcggccaaaaaatccctggccgaacgtctgtcagcactgctgaatg
ttgatccgacaaaatgaaaggcggtggcggctcaggtggcggtggctctggtggcggtggttcaggcgtcaaagaaagtctggtgtga
CmCHI-Citrus maxima
Amino acid sequence
(SEQ ID NO: 41)
MNPSPSVTELQVENVTFTPSLQPPGSTKSHFLGGAGERGLEIEGKFVKFTAIGVYLEENA
VPLLAGKWKGKTAGELTESVEFFRDVVTGPFEKFMKVTMILPLTGAQYSEKVAENCMA
IWKFFGIYTDAEAKAIEKFTEVFKDEIFPPGSSILFTQSSGSLTISFSKDGSIPKDGVAVIE
NNLLSEAVLESMIGKNGVSPAAKKSLAERLSALLNVASDKMKGGGGSGGGGSGGGGS
GVKESLV
MsCHI-Medicago sativa
Nucleic acid sequence
(SEQ ID NO: 42)
atggctgcatcaatcaccgcaatcactgtggagaaccttgaatacccagcggtggttacctctccggtcaccggcaaatcatatttcctcggt
ggcgctggggagagaggattgaccattgaaggaaacttcatcaagttcactgccataggtgtttatttggaagatatagcagtggcttcacta
gctgccaaatggaagggtaaatcatctgaagagttacttgagacccttgacttttacagagacatcatctcaggtccctttgaaaagttaattag
agggtcaaagattagggaattgagtggtcctgagtactcaaggaaggttatggagaactgtgtggcacacttgaaatcagttggaacttatgg
agatgcagaagctgaagctatgcaaaaatttgctgaagctttcaagcctgttaattttccacctggtgcctctgttttctacaggcaatcacctaa
tggaatattagggcttagtttctctccggatacaagtataccagaaaaggaggctgcactcatagagaacaaggcagtttcatcagcagtgtt
ggagactatgatcggcgagcacgctgtttcccctgatcttaagcgctgtttagctgcaagattacctgcgttgttgaacgagggtgctttcaag
attggaaactga
MsCHI-Medicago sativa
Amino acid sequence
(SEQ ID NO: 43)
MAASITAITVENLEYPAVVTSPVTGKSYFLGGAGERGLTIEGNFIKFTAIGVYLEDIAVA
SLAAKWKGKSSEELLETLDFYRDIISGPFEKLIRGSKIRELSGPEYSRKVMENCVAHLKS
VGTYGDAEAEAMQKFAEAFKPVNFPPGASVFYRQSPNGILGLSFSPDTSIPEKEAALIEN
KAVSSAVLETMIGEHAVSPDLKRCLAARLPALLNEGAFKIGN
CsF3H-Camellia sinensis
Nucleic acid sequence
(SEQ ID NO: 44)
atggcaccgaccaccaccctgaccgcactggcagaagaaaaaagcctgcagcagaaatttgttcgtgatgaagatgaacgtccgaaagtt
gcctataatgtgtttagcaatgaaatcccggttattagcctggcaggtattgatgaaattgaaggtcgtcgtagcgaaatttgccgtaaaattgtt
gaagcatgtgaaggttggggtgtttttcaggttgttgatcatggtgttgatgcaaatctgattgcagaaatgacccgtctggcacgtgaatttttt
gcactgcctccggaagaaaaactgcgttttgatatgagcggtggtaaaaaaggtggttttattgttagcagccatctgcagggtgaagcagtt
caggattggcgtgaaattgttacctatttcagctatccgattcgtgcacgtgattatagccgttggcctgataaaccggaaggttggcgtgcag
ttaccgaaacctatagcgaaaaactgatggatctggcatgtaaactgctggaagttctgagcgaagcaatgggtctggaaaaagaggcact
gaccaaagcatgtgttgatatggatcagaaagtggtgatcaacttctatccgaaatgtccgcagccggatctgaccctgggtctgaaacgtca
taccgatccgggtacaattaccctgctgctgcaagatcaggtgggtggtctgcaggcaacccgtgatggtggcaaaacctggattaccgttc
agccggttgaaggtgcatttgttgttaatctgggtgatcatggccattatctgagcaatggtcgctttaaaaacgcagatcatcaggcagttgtt
aatagcaattgtagccgtctgagcattgcaacctttcagaatccggcaccggaagcaaccgtttatccgctgaaaattcgtgaaggtgaaaaa
ccgattctggaagaaccgattacctttgccgatatgtataaacgcaaaatgagcaaagatatcgagctggccaaactgaaaaaactggcgaa
agaaaaaaaactgctgcaagaccagcaggatatcgaaaaagcaaaactggaaatcaaaagcaccgatgaaatcttcgccctggttggtgc
actgatgcatgttatgcagaaacgtagccgtgcaattcatagcagtgatgaaggtgaagatcaagccggtgatgaagatgaggat
CsF3H-Camellia sinensis
Amino acid sequence
(SEQ ID NO: 45)
MAPTTTLTALAEEKSLQQKFVRDEDERPKVAYNVFSNEIPVISLAGIDEIEGRRSEICRKIV
EACEGWGVFQVVDHGVDANLIAEMTRLAREFFALPPEEKLRFDMSGGKKGGFIVSSHL
QGEAVQDWREIVTYFSYPIRARDYSRWPDKPEGWRAVTETYSEKLMDLACKLLEVLSE
AMGLEKEALTKACVDMDQKVVINFYPKCPQPDLTLGLKRHTDPGTITLLLQDQVGGLQ
ATRDGGKTWITVQPVEGAFVVNLGDHGHYLSNGRFKNADHQAVVNSNCSRLSIATFQN
PAPEATVYPLKIREGEKPILEEPITFADMYKRKMSKDIELAKLKKLAKEKKLLQDQQDIE
KAKLEIKSTDEIFALVGALMHVMQKRSRAIHSSDEGEDQAGDEDED
MdF3H-Malus domestica
Nucleic acid sequence
(SEQ ID NO: 46)
atggcaccgcctgcaaccaccctgaccagcattgcacatgaaaaaaccctgcagcagaaatttgttcgtgatgaagatgaacgtccgaaag
tggcctataatgaatttagcaacgaaatcccgattattagcctggcaggtattgatgaagttgaaggtcgtcgtgccgaaatctgcaaaaaaat
cgttgaagcatgtgaagattggggcatttttcagattgttgatcatggtgttgatgccgaactgattagcgaaatgaccggtctggcaaaagaa
ttttttgatctgccgagcgaagaaaaactgcgttttgatatgagcggtggtaaaaaaggtggttttattgttagcagccatctgcagggtgaagc
agttcaggattggcgtgaaattgttacctattttctgtatccgattcgccaccgtgattatagccgttggcctgataaaccggaagcatggcgtg
aagttaccaaaaaatacagtgatgaactgatgggtctggcatgtaaactgctgggtgttctgagcgaagcaatgggcctggataccgaagc
actgaccaaagcatgtgttgatatggatcagaaagtggtggttaacttctatccgaaatgtccgcagccggatctgaccctgggtctgaaacg
tcataccgatccgggtacaattaccctgctgctgcaagatcaggttggcggtctgcaggcaacccgtgatgatggtaaaacctggattaccg
ttcagccggttgaaggtgcatttgttgttaatctgggtgatcatggccattttctgagcaatggtcgctttaaaaacgcagatcatcaggcagttg
ttaatagcaatagcagccgtctgagcattgcaacctttcagaatccggcacaggatgcaattgtttatccgctgagcgttcgtgaaggtgaaa
aaccgattctggaagcaccgattacctataccgagatgtataaaaaaaaaatgagcaaagatctggaactggcacgcctgaaaaaactggc
caaagaacagcagctgcaggatctggaaaaagcaaaagttgaaaccaaaccggcagatgatatctttgccctggttggtgcactgatgcat
gttatgcagaaacgtagccgtgcaattcatagcagtgatgaaggtgaagatcaagccggtgatgaagatgaggat
MdF3H-Malus domestica
Amino acid sequence
(SEQ ID NO: 47)
MAPPATTLTSIAHEKTLQQKFVRDEDERPKVAYNEFSNEIPIISLAGIDEVEGRRAEICKKI
VEACEDWGIFQIVDHGVDAELISEMTGLAKEFFDLPSEEKLRFDMSGGKKGGFIVSSHLQ
GEAVQDWREIVTYFLYPIRHRDYSRWPDKPEAWREVTKKYSDELMGLACKLLGVLSEA
MGLDTEALTKACVDMDQKVVVNFYPKCPQPDLTLGLKRHTDPGTITLLLQDQVGGLQ
ATRDDGKTWITVQPVEGAFVVNLGDHGHFLSNGRFKNADHQAVVNSNSSRLSIATFQN
PAQDAIVYPLSVREGEKPILEAPITYTEMYKKKMSKDLELARLKKLAKEQQLQDLEKAK
VETKPADDIFALVGALMHVMQKRSRAIHSSDEGEDQAGDEDED
PcF3H-Petroselinum crispum
Nucleic acid sequence
(SEQ ID NO: 48)
atggcaccgagcaccctgaccgcactggcacaagaaaaaaccctgaatagcaaatttgtgcgcgacgaagatgaacgtccgaaaattgca
tataacaaattcagcgacgaaatcccggttattagcctggcaggtattgatgatgatagcgttgataaacgtagccagatttgccgtaaaattgt
tgaagcatgtgaagattggggcatttttcaggttgttgatcatggcattgatatcgatctgattagcgaaatgacccgtctggcacgtcagtttttt
gcactgcctgcagaagaaaaactgcgttttgatatgaccggtggtaaaaaaggtggttttattgttagcagccatctgcagggtgaagcagtt
caggattggcgtgaaattgttacctatttcagctatccgattcaggcacgtgattatagccgttggcctgataaaccggaaggttggcgtagca
ttaccgaaatgtatagtgatgaactgatggcactggcatgtaaactgctggaagttctgagcgaagcaatgggtctggaaaaagagggtctg
accaaagcatgtgttgatatggatcagaaagtgatcgtgaactactatccgaaatgtccgcagccgaatctgaccctgggtctgaaacgtcat
accgatccgggtacaattaccctgctgctgcaggatcaggttggtggtctgcaggcgacccgtgatggtggcaaaacctggattaccgttca
gccggttgaaggtgcatttgttgttaatctgggtgatcatggtcactatctgagcaatggtcgctttaaaaacgcagatcatcaggcagttgtta
atagcaatagcagccgtatgagcattgcaacctttcagaatccggcaccgaatgcaaccgtttatccgctgaaaattcgtgaaggtgaaaaa
gccgttatggaagaaccgattacctttgccgagatgtataaacgtaaaatgagccgtgatattgaaatggccaccctgaaaaaactggccaa
agaaaaagttctgcaggaccaagaagtggaaaaagcaaaactgcagatgaccccgaaaagcgcagatgaaatttttgccctggttggtgc
actgatgcatgttatgcagaaacgtagccgtgcaattcatagcagtgatgaaggtgaagatcaagccggtgatgaagatgaggat
PcF3H-Petroselinum crispum
Amino acid sequence
(SEQ ID NO: 49)
MAPSTLTALAQEKTLNSKFVRDEDERPKIAYNKFSDEIPVISLAGIDDDSVDKRSQICRK
IVEACEDWGIFQVVDHGIDIDLISEMTRLARQFFALPAEEKLRFDMTGGKKGGFIVSSHL
QGEAVQDWREIVTYFSYPIQARDYSRWPDKPEGWRSITEMYSDELMALACKLLEVLSE
AMGLEKEGLTKACVDMDQKVIVNYYPKCPQPNLTLGLKRHTDPGTITLLLQDQVGGLQ
ATRDGGKTWITVQPVEGAFVVNLGDHGHYLSNGRFKNADHQAVVNSNSSRMSIATFQ
NPAPNATVYPLKIREGEKAVMEEPITFAEMYKRKMSRDIEMATLKKLAKEKVLQDQEV
EKAKLQMTPKSADEIFALVGALMHVMQKRSRAIHSSDEGEDQAGDEDED
AaDFR-Anthurium andraeanum
Nucleic acid sequence
(SEQ ID NO: 50)
atgatgcataaaggcaccgtttgtgttaccggtgcagcaggttttgttggtagctggctgattatgcgtctgctggaacagggttatagcgttaa
agcaaccgttcgtgatccgagcaatatgaaaaaagttaaacatctgctggatctgcctggtgcagcaaatcgtctgaccctgtggaaagcag
atctggttgatgaaggtagattgatgaaccgattcagggttgtaccggtgtttttcatgttgcaaccccgatggattttgaaagcaaagatccg
gaaagcgaaatgattaaaccgaccattgaaggtatgctgaatgttctgcgtagctgtgcccgtgcaagcagcaccgttcgtcgtgttgttttta
ccagcagcgcaggtacagttagcattcatgaaggtcgtcgtcatctgtatgatgaaaccagttggagtgatgttgatttttgccgtgccaaaaa
aatgaccggctggatgtattttgttagcaaaaccctggcagaaaaagcagcatgggattttgcagagaaaaataacatcgacttcatcagcat
tattccgaccctggttaatggtccgtttgttatgccgaccatgcctccgagcatgctgagcgcactggcactgattacccgtaatgaaccgcat
tatagcattctgaatccggtgcagtttgttcatctggatgatctgtgtaacgcccacatttttctgtttgaatgtccggatgcaaaaggtcgttatat
ttgtagcagccatgatgttaccattgcaggtctggcacagattctgcgtcagcgttatccggaatttgatgttccgaccgaatttggtgaaatgg
aagtgtttgatatcatcagctatagcagcaaaaaactgacggatctgggtttcgaattcaaatatagcctggaagatatgttcgatggtgcaatt
cagagctgtcgtgaaaaaggtctgctgcctccggcaaccaaagaaccgagctatgcaaccgaacagctgattgcaaccggtcaggataat
ggtcatcctcctcctgcactgcctccgaaacgtcgtcgt
AaDFR-Anthurium andraeanum
Amino acid sequence
(SEQ ID NO: 51)
MMHKGTVCVTGAAGFVGSWLIMIRLLEQGYSVKATVRDPSNMKKVKHLLDLPGAANR
LTLWKADLVDEGSFDEPIQGCTGVEHVATPMDFESKDPESEMIKPTIEGMLNVLRSCAR
ASSTVRRVVFTSSAGTVSIHEGRRHLYDETSWSDVDFCRAKKMTGWMYFVSKTLAEKA
AWDFAEKNNIDFISIIPTLVNGPFVMPTMPPSMLSALALITRNEPHYSILNPVQFVHLDDL
CNAHIFLFECPDAKGRYICSSHDVTIAGLAQILRQRYPEEDVPTEFGEMEVEDIISYSSKK
LTDLGFEFKYSLEDMFDGAIQSCREKGLLPPATKEPSYATEQLIATGQDNGHPPPALPPK
RRR
CsDFR-Camellia sinensis
Nucleic acid sequence
(SEQ ID NO: 52)
atgaaagatagcgttgcaagcgcaaccgcaagcgcaccgggtacagtttgtgttaccggtgcagcaggttttattggtagctggctggttatg
cgtctgctggaacgtggttatattgttcgtgcaaccgttcgtgatccggcaaatctgaaaaaagttaaacatctgctggatctgccgaaagcag
ataccaatctgaccctgtggaaagccgatctgaatgaagagggtagctttgatgaagcaattgaaggttgtagcggtgtttttcatgttgcaac
cccgatggattttgaaagcaaagatccggaaaacgaagtgattaaaccgaccattaacggtgtgctgagcattattcgtagctgtaccaaag
caaaaaccgttaaacgtctggtttttaccagcagcgcaggtacagttaatgttcaagaacatcagcagccggtgtttgatgaaaacaattgga
gcgatctgcacttcatcaacaaaaaaaaaatgaccggctggatgtattttgtgagcaaaaccctggcagaaaaagcagcatgggaagcagc
aaaagaaaacaacattgatttcatcagcattatcccgaccctggttggtggtccgtttattatgccgacctttccgcctagcctgattaccgcact
gagcccgattacccgtaatgaaggtcattattccattatcaaacagggccagtttgtgcatctggatgatctgtgtgaaagccacatttttctgta
tgaacgtccgcaggcagaaggtcgttatatttgtagcagccatgatgcaaccattcatgatctggccaaactgatgcgtgaaaaatggcctga
atataatgttccgaccgaattcaaaggcatcgataaagatctgccggttgttagcttttccagcaaaaaactgattggcatgggcttcgagttca
aatatagcctggaagatatgtttcgtggtgccattgatacctgtcgtgaaaaaggtctgctgccgcatagctttgcagaaaatccggttaatgg
caacaaagtgcctcctcctgcactgcctccgaaacgtcgtcgt
CsDFR-Camellia sinensis
Amino acid sequence
(SEQ ID NO: 53)
MKDSVASATASAPGTVCVTGAAGFIGSWLVMRLLERGYIVRATVRDPANLKKVKHLL
DLPKADTNLTLWKADLNEEGSFDEAIEGCSGVFHVATPMDFESKDPENEVIKPTINGVLS
IIRSCTKAKTVKRLVFTSSAGTVNVQEHQQPVFDENNWSDLHFINKKKMTGWMYFVSK
TLAEKAAWEAAKENNIDFISIIPTLVGGPFIMPTFPPSLITALSPITRNEGHYSIIKQGQFVH
LDDLCESHIFLYERPQAEGRYICSSHDATIHDLAKLMREKWPEYNVPTEFKGIDKDLPVV
SFSSKKLIGMGFEEKYSLEDMERGAIDTCREKGLLPHSFAENPVNGNKVPPPALPPKRRR
FaDFR-Fragaria x ananassa N
ucleic acid sequence
(SEQ ID NO: 54)
atgggtctgggtgcagaaagcggtagcgtttgtgttaccggtgcaagcggttttgttggtagctggctggttatgcgtctgctggaacatggtt
ataccgttcgtgcaaccgtgcgtgatccggcaaatctgaaaaaagttcgtcatctgctggaactgccgcaggcagcaacccgtctgaccctg
tggaaagcagatctggatgttgaaggtagattgatgaagccattaaaggttgtaccggtgtttttcatgttgcaaccccgatggattttgaaag
cgaagatccggaaaacgaagttattaaaccgaccattaacggcatgctggatattatgaaagcatgcctgaaagcaaaaaccgttcgtcgtc
tggtttttaccagcagtgccggtgcagttgcaattgaagaacatccgaaagaagtgtacagcgaaaataactggtcagatgttgtgttttgccg
caaagttaaaatgaccggctggatgtattttgtgagcaaaaccctggcagaacaggcagcatggaaatttgcaaaagaaaacaacatcgac
ttcatcaccattattccgaccctggttattggtccgtttctggcaccgagcatgcctccgagcctgattagcggtctgagtccgctgaccggtaa
tgaagcacattatggtattatcaaacagtgccagtatgtgcatctggatgatctgtgtcagagccatatttttctgtatgaacatgcaaaagccga
gggtcgttatatttgtagcagccatgatgcaaccattcacgatattgcaaaactgctgaacgagaaatacccgaaatacaacgttccgaaaaa
attcaaaggcatcgaagaaaacctgaccaacattcactttagcagcaaaaaactgaaagagatgggcttcgaatttaaacacagcctggaa
gatatgtttacaggtgccgttgatgcatgtcgtgaaaaaggtctgctgccgctgccgcaagaagaagaaaccgaaaaacgtcgtgcaggtc
ctcctcctgcactgcctccgaaacgtcgtcgt
FaDFR-Fragaria x ananassa
Amino acid sequence
(SEQ ID NO: 55)
MGLGAESGSVCVTGASGFVGSWLVMRLLEHGYTVRATVRDPANLKKVRHLLELPQAA
TRLTLWKADLDVEGSFDEAIKGCTGVFHVATPMDFESEDPENEVIKPTINGMLDIMKAC
LKAKTVRRLVFTSSAGAVAIEEHPKEVYSENNWSDVVFCRKVKMTGWMYFVSKTLAE
QAAWKFAKENNIDFITIIPTLVIGPFLAPSMPPSLISGLSPLIGNEAHYGIIKQCQYVHLDD
LCQSHIFLYEHAKAEGRYICSSHDATIHDIAKLLNEKYPKYNVPKKFKGIEENLTNIHFSS
KKLKEMGFEEKHSLEDMFTGAVDACREKGLLPLPQEEETEKRRAGPPPALPPKRRR
CsLAR-Camellia sinensis
Nucleic acid sequence
(SEQ ID NO: 56)
atggcaatggccatggcaaccaccaccacaaccaccaaaccgatgattggtgcaaaagcagcatgtgttgttggtggcaccggttttgttgc
agcaaccctggttaaaatgctgctggaacgtggttatagcgttaataccaccgttcgtgatccggacaacaaaaaaaacattagccatctggt
tgcactggaaggtatgggtaatctgaaaatctttcgtgcagatctgaccgatgaacagagctttgatgcaccgattgcaggttgtgatctggttt
ttgatgttgccacaccggttaattttgcaagcgaagatccggaaaacgacatgattaaactggcaattcagggtgttctgaatgtgctgaaagc
atgtgccaaagcaggcaccgttaaacgtgttattctgaccagcagcgcagcaagcgttaccattaatcagctggatggtacaggtctggttat
ggatgaaagccattggagtgatgttgaatttctgacctcagttaaaccgcctacctggggtcatccggttagcaaaaccctggcagaaaaag
cagcctggaaatttgcagaagaaaataacctgaatctgattaccgttgttccgaccctgaccgcaggtccgagcctgaccagcgaagttccg
aatagcattgaactggccatgagcctgattacgggtaatgaattcctgattgatggtctgaaaggtatgcgtattctgtcaggtagcattagcat
tacccatgttgaagatgtttgtggtgcccatatttttgtggccgaaaaagaaagcgcaagcggtcgttatatttgttgtggtgttaatagcagcgt
gccggaactggcacgttttctgaataaacgttatccgcagtataatgtgccgaccgattttggtgatctgccgagcaaagcaaaactgattatt
agcagcgagaaactgatcaaagaaggatcagatcaaatatggcatcgaagaaatttttgcacacagcgttgcatatctgaaaaccaaagg
tctgctgcagaacggtgttaaagaaagcctggtt
CsLAR-Camellia sinensis
Amino acid sequence
(SEQ ID NO: 57)
MAMAMATTTTTTKPMIGAKAACVVGGTGEVAATLVKMLLERGYSVNTTVRDPDNKK
NISHLVALEGMGNLKIFRADLTDEQSFDAPIAGCDLVEDVATPVNEASEDPENDMIKLAI
QGVLNVLKACAKAGTVKRVILTSSAASVTINQLDGTGLVMDESHWSDVEFLTSVKPPT
WGHPVSKTLAEKAAWKFAEENNLNLITVVPTLTAGPSLTSEVPNSIELAMSLITGNEFLI
DGLKGMRILSGSISITHVEDVCGAHIEVAEKESASGRYICCGVNSSVPELARFLNKRYPQ
YNVPTDFGDLPSKAKLIISSEKLIKEGFSFKYGIEEIFAHSVAYLKTKGLLQNGVKESLV
DuLAR-Desmodium uncinatum
ucleic acid sequence
(SEQ ID NO: 58)
atgaccgttagcggtgcaattccgagcatgaccaaaaatcgtaccctggttgttggtggcaccggttttattggtcagtttattaccaaagcaa
gcctgggttttggttatccgacctttctgctggttcgtccgggtccggttagcccgagcaaagcagttattatcaaaacctttcaggataaaggt
gccaaagtgatttatggcgtgatcaacgataaagaatgcatggaaaaaattctgaaagagtacgagatcgacgttgttattagcctggtgggt
ggtgcacgtctgctggatcagctgaccctgctggaagcaattaaaagcgttaaaaccatcaaacgttttctgccgagcgaatttggccatgat
gttgatcgtaccgatccggttgaaccgggtctgaccatgtataaagaaaaacgtctggtgcgtcgtgccgttgaagaatatggtattccgttta
ccaatatctgctgcaatagcattgcaagctggccgtattatgataattgtcatccgagccaggttccgcctccgatggatcagtttcagatttatg
gtgatggtaacaccaaagcctatttcattgatggcaacgatatcggcaaatttaccatgaaaaccatcgatgatattcgcaccctgaacaaaaa
tgttcattttcgtccgagcagcaactgctacagcattaatgaactggcaagcctgtgggagaaaaaaatcggtcgtacactgcctcgttttacc
gttaccgcagataaactgctggcacatgcagcagaaaacattattccggaaagcattgttagcagctttacccacgatatctttattaacggttg
ccaggtgaactttagcatcgatgaacatagtgatgtggaaatcgatacactgtatccggatgaaaaatttcgtagcctggatgattgctatgaa
gattttgttccgatggtgcacgataaaattcatgcaggtaaaagcggtgaaatcaaaatcaaagatggtaaaccgctggttcagaccggcac
cattgaagaaattaacaaagacattaaaaccctggtggaaacccagccgaatgaagagatcaaaaaagatatgaaagcactggttgaagcc
gttccgattagcgcaatgggtggtgttaaagaaagcctggtt
DuLAR-Desmodium uncinatum
Amino acid sequence
(SEQ ID NO: 59)
MTVSGAIPSMTKNRTLVVGGTGFIGQFITKASLGFGYPTFLLVRPGPVSPSKAVIIKTFQ
DKGAKVIYGVINDKECMEKILKEYEIDVVISLVGGARLLDQLTLLEAIKSVKTIKRFLPS
EFGHDVDRTDPVEPGLTMYKEKRLVRRAVEEYGIPFTNICCNSIASWPYYDNCHPSQVP
PPMDQFQIYGDGNTKAYFIDGNDIGKFTMKTIDDIRTLNKNVHFRPSSNCYSINELASLW
EKKIGRTLPRFTVTADKLLAHAAENIIPESIVSSFTHDIFINGCQVNFSIDEHSDVEIDTL
YPDEKFRSLDDCYEDFVPMVHDKIHAGKSGEIKIKDGKPLVQTGTIEEINKDIKTLVETQ
PNEEIKKDMKALVEAVPISAMGGVKESLV
PhANS-Petunia X hybrida
Nucleic acid sequence
(SEQ ID NO: 60)
atggtgaatgcagtagttacaactccttcaagagttgaaagcttggctaaaagtggaatccaggccatccctaaggagtatgtgaggccaca
agaagagttgaatggaatcggaaacatcttcgaggaagagaagaaagatgaagggcctcaagtaccaacaattgatttgaaagaaattgac
tccgaggacaaggagattcgcgagaaatgccaccaggagttgaagaaagcagccatggaatggggtgtcatgcaccttgtgaatcatggc
atatccgatgagctaatcaatcgtgtcaaggttgctggagagaccttctttgatcaacctgttgaagaaaaggagaagtatgctaatgaccaa
gccaatggcaatgtccaaggctacggcagcaagctagcaaatagtgcttgtggtcagcttgagtgggaggattatttcttccattgtgctttcc
ctgaagacaagcgcgacttgtccatctggcctaaaaatcctactgactacactccagcaacaagtgaatatgccaagcagatcagggccct
agcaacaaagattttgacagtgctttctattgggctggggctggaagaaggaagactagagaaggaagttggaggcatggaggatctgctg
cttcaaatgaagattaactactatcccaagtgcccccaaccagaactagcacttggcgtcgaagctcatacagatgtcagcgcactgactttc
atcctccacaatatggtgcccggcttgcaactcttctatgaaggccagtgggtaactgctaagtgtgtgcctaattctatcatcatgcacatagg
ggacaccattgaaatcctaagcaatggaaagtacaagagcatccttcatagaggggttgtgaataaagagaaagtaaggatctcatgggcc
attttctgcgagccacctaaggagaagatcatccttaagcccctacctgagactgtcactgaggctgagccacctcgattcccacctogcacc
tttgcacagcatatggcacacaagctcttcaggaaggatgacaaggatgccgctgttgaacacaaagtcttcaaagaggatgaactggatac
tgctgctgaacataaggtcctcaagaaggataatcaggatgctgttgctgagaataaagacatcaaggaggatgaacagtgtggccctgct
gagcacaaagatatcaaggaggatggacagggtgccgctgctgagaacaaagtcttcaaggagaataatcaggatgttgctgctgaagaa
tctaaatag
PhANS-Petunia X hybrida
Amino acid sequence
(SEQ ID NO: 61)
MVNAVVTTPSRVESLAKSGIQAIPKEYVRPQEELNGIGNIFEEEKKDEGPQVPTIDLKEI
DSEDKEIREKCHQELKKAAMEWGVMHLVNHGISDELINRVKVAGETFFDQPVEEKEKY
ANDQANGNVQGYGSKLANSACGQLEWEDYFFHCAFPEDKRDLSIWPKNPTDYTPATSE
YAKQIRALATKILTVLSIGLGLEEGRLEKEVGGMEDLLLQMKINYYPKCPQPELALGVE
AHTDVSALTFILHNMVPGLQLFYEGQWVTAKCVPNSIIMHIGDTIEILSNGKYKSILHRG
VVNKEKVRISWAIFCEPPKEKIILKPLPETVTEAEPPRFPPRTFAQHMAHKLFRKDDKDA
AVEHKVFKEDELDTAAEHKVLKKDNQDAVAENKDIKEDEQCGPAEHKDIKEDGQGAA
AENKVFKENNQDVAAEESK
At3GT-Arabidopsis thaliana
Nucleic acid sequence
(SEQ ID NO: 62)
atgaccaaaccctccgacccaaccagagactcccacgtggcagttctcgcttttcctttcggcactcatgcagctcctctcctcaccgtcacg
cgccgcctcgcctccgcctctccttccaccgtcttctctttcttcaacaccgcacaatccaactcttogttattttcctccggtgacgaagcagat
cgtccggcgaacatcagagtatacgatattgccgacggtgttccggagggatacgtgtttagcgggagaccacaggaggcgatcgagctg
tttcttcaagctgcgccggagaatttccggagagaaatcgcgaaggcggagacggaggttggtacggaagtgaaatgtttgatgactgatg
cgttatctggttcgcggctgatatggcgacggagataaatgcgtcgtggattgcgttttggaccgccggagcaaactcactctctgctcatct
ctacacagatctcatcagagaaaccatcggtgtcaaagaagtaggtgagcgtatggaggagacaataggggttatctcaggaatggagaa
gatcagagtcaaagatacaccagaaggagttgtgtttgggaatttagactctgttttctcaaagatgcttcatcaaatgggtatgctttgcctcg
tgccactgctgttttcatcaattcttttgaagatttggatcctacattgacgaataacctcagatcgagatttaaacgatatctgaacatcggtcctc
tcgggttattatcttctacattgcaacaactagtgcaagatcctcacggttgtttggcttggatggagaagagatcttctggttctgtggcgtacat
tagattggtacggtcatgacaccgcctcctggagagatgcggcgatagcagaagggttggaatcgagtaaagtgccgtttgtttggtcgct
taaggagaagagcttggttcagttaccaaaagggtttttggataggacaagagagcaagggatagtggttccatgggcaccgcaagtggaa
ctgctgaaacacgaagcaacgggtgtgtttgtgacgcattgtggatggaactcggtgttggagagtgtatcgggtggtgtaccgatgatttgc
aggccattttttggggatcagagattgaacggaagagcggtggaggttgtgtgggagattggaatgacgattatcaatggagtatcacgaa
agatgggtttgagaagtgtttggataaagttttagttcaagatgatggtaagaagatgaaatgtaatgctaagaaacttaaagaactagcttacg
aagctgtctcttctaaaggaaggtcctctgagaatttcagaggattgttggatgcagttgtaaacattatttga
At3GT-Arabidopsis thaliana
Amino acid sequence
(SEQ ID NO: 63)
MTKPSDPTRDSHVAVLAFPFGTHAAPLLTVTRRLASASPSTVFSFFNTAQSNSSLFSSGD
EADRPANIRVYDIADGVPEGYVFSGRPQEAIELFLQAAPENFRREIAKAETEVGTEVKCL
MTDAFFWFAADMATEINASWIAFWTAGANSLSAHLYTDLIRETIGVKEVGERMEETIGV
ISGMEKIRVKDTPEGVVFGNLDSVFSKMLHQMGLALPRATAVFINSFEDLDPTLTNNLRS
RFKRYLNIGPLGLLSSTLQQLVQDPHGCLAWMEKRSSGSVAYISFGTVMTPPPGELAAIA
EGLESSKVPFVWSLKEKSLVQLPKGFLDRTREQGIVVPWAPQVELLKHEATGVFVTHCG
WNSVLESVSGGVPMICRPFFGDQRLNGRAVEVVWEIGMTIINGVFTKDGFEKCLDKVLV
QDDGKKMKCNAKKLKELAYEAVSSKGRSSENFRGLLDAVVNII
Fragaria x ananassa 3GT
Amino acid sequence
(SEQ ID NO: 64)
MGSAVAVELVFIPAPGVGHIMSTMEMAKLLINRHQSIATTVLLIHPPYSSSVLTNYIQSLL
TNPIQRIRFIQLPQDQETASKLDLKAPFTSFYEFINSHRNYVRNVVSDMLSRPGSVRITGL
VVDILCTGMIDVANEFSIPSYAFFTSNAAFLGFKLYMDTLCRNQKQEGIIALSKSDGELRI
PSFVKPVPMTVYPAVYQTRDGLDFLTVSIQKFREAKAIMVNTFLELETHAIESFSSYTNFP
SVYAVGPVLNLNGVAGKDEDKDVIRWLDGQPPSSVVFLCFGSMGSFEEVQLKEIAYAL
ERSGHRFVWSVRRPPSPEQSFKVLPDDYDDPRSILPDGFLERTNGFGKVIGWAPQVSILA
HEAVGGFVSHCGWNSVLESICCKVPILAWPMMAEQHLNARMVVEEIKIGLRVETCDGS
VRGFVQADGLKKMVKELMEGENGEIVRKRVEGIGEGAKKAMAEGGSSWRTLNELIDE
LQCVRNSNGGRFPSSEGDSDKSKGESYVPMDNLSLVSI
Vitis vinifera 3GT
Amino acid sequence
(SEQ ID NO: 65)
MSQTTTNPHVAVLAFPFSTHAAPLLAVVRRLAAAAPHAVFSFFSTSQSNASVFHDSMHT
MQCNIKSYDVSDGVPEGYVFAGRPQEDIELFMRAAPEGFRQGMVMAVAETGRPVSCLV
ADAFIWFAADMAAEMGVAWLPFWTAGPNSLSTHVYTDEIREKIGVSGIQGREDELLNFI
PGMYEVRFRDLQEGIVFGNLNSLFSRMLHRMGQVLPKATAVFINSFEELDDSLTNDLKS
KLKTYLNIGPFNLITPPPVVPNTTGCLQWLKERKPTSVVYISFGTVTTPPPAELVALAEAL
EASRVPFIWSLRDKARVHLPEGFLEKTRGYGMVVPWAPQAEVLAHEAVGAFVTHCGW
NSLWESVAGGVPLICRPFFGDQRLNGRMVEDVLEIGVRIEGGVFTKSGLMSCFDQILSQE
KGKKLRENLRALRETADRAVGPKGSSTENFKTLVDLVSKPKDV
Forsynthia 3GT
Amino acid sequence
(SEQ ID NO: 66)
MAIHSHIGVLAFPFGTHAAPLLTLVRRLVLDSSSQGITFSFFNTAKSNCAIFSGQEFDNIKA
YDVWDGTHEGEAFTGSNILEAMQLFLAATPGNFEKVMKEAEVKNGMKISCLLSDAFLW
FTCDLAEERGIPWVSFWTAASCSLSAHMYTDQIWSLMRSTGTAKTEEKTLSFVPGMTSV
RFSDLPEEILSDNLESPLTLMIYKMVQKLSKSTAIVVNSFEEIDPVITNDLKSKFQNFLNIG
PSILSSPTLSNGDSGQECLLWLEKQRHASVIYISFGTVITPQPREMAGLAEALETGEFPFL
WSLRDNAMKLLPDGFLDRTSKFGMIVSWAPQLKVLENPSVGAFITHCGWNSILESISFG
VPMICRPFFGDQNLNSKMVEDVWKIGVRLEGGVFTKNGTIEALHSVMLNETGKAIRENI
NKLKRKAQNAVKFDGTSTKNFRALLELIKSPRGI
Eggplant 3GT
Amino acid sequence
(SEQ ID NO: 67)
MTTSQLHIAFLAFPFGTHATPLLTLVQKISPFLPSSTIFSFFNTSSSNSSIFSKVPNQENIKIY
NVWDGVKEGNDTPFGLEAIKLFIQSTLLISKITEEAEEETGVKFSCIFSDAFLWCFLVKLP
KKMNAPGVAYWTGGSCSLAVHLYTDLIRSNKETSLKIPGFSSTLSINDIPPEVTAEDLEGP
MSSMLYNMALNLHKADAVVLNSFQELDRDPLINKDLQKNLQKVFNIGPLVLQSSRKLD
ESGCIQWLDKQKEKSVVYLSFGTVTTLPPNEIGSIAEALETKKTPFIWSLRNNGVKNLPK
GFLERTKEFGKIVSWAPQLEILAHKSVGVFVTHCGWNSILEGISFGVPMICRPFFGDQKL
NSRMVESVWEIGLQIEGGIFTKSGIISALDTFFNEEKGKILRENVEGLKEKALEAVNQMM
EVQQKISRF
Gentian 3 GT
Amino acid sequence
(SEQ ID NO: 68)
MDQLHVFFFPFLANGHILPTIDMAKLFSSRGVKATLITTHNNSAIFLKAINRSKILGFDISV
LTIKFPSAEFGLPEGYETADQARSIDMMDEFFRACILLQEPLEELLKEHRPQALVADLFFY
WANDAAAKFGIPRLLFHGSSSFAMIAAESVRRNKPYKNLSSDSDPFVVPDIPDKIILTKSQ
VPTPDETEENNTHITEMWKNISESENDCYGVIVNSFYELEPDYVDYCKNVLGRRAWHIG
PLSLCNNEGEDVAERGKKSDIDAHECLNWLDSKNPDSVVYVCFGSMANFNAAQLHELA
MGLEESGQEFIWVVRTCVDEEDESKWFPDGFEKRVQENNKGLIIKGWAPQVLILEHEAV
GAFVSHCGWNSTLEGICGGVAMVTWPLFAEQFYNEKLMTDILRTGVSVGSLQWSRVTT
SAVVVKRESISKAVRRLMAEEEGVDIRNRAKALKEKAKKAVEGGGSSYSDLSALLVELS
SYPHN
Petunia x hybrida 3GT
Amino acid sequence
(SEQ ID NO: 69)
MTTSQLHIALLAFPFGSHAAPLLTLVQKLSPFLPSDTIFSFFNTSQSNTSIFSEGSKPDNIKV
YNVWDGVTETNGNKPVGLEAIKLFIQATPTNFEKVMKEAEEETGVKFSCIFSDAFLWFS
YKLAEKINVPWIAFWTAASGSLSVHLYTDFIRSNDETSLNIPGFSSTLKISDMPPEVMAEN
LDLPMPSMLYNMALNLHKAAAVVLNSFEELDPTINKDLKVKLQKVLNIGPLVLQPTSPK
KVLDACDERGCIIWLEKQKEESVVYLSFGTVTTLPPNEIVAVAEALEAKKFPFIWSLKDN
GIKNLPTGFLERTGQFGKIVSWAPQLEILNHSAVGVFVTHCGWNSILEGISCGVPMICRPF
FGDQKLNSRMVESVWQIGLQIEGGSFTKIGTISALDTFFSEEKGKVLRENVKGLKERALE
AVKPDGSSSKNFKDLVELVKCHKLT
Malus domestica ANS
Amino acid sequence
(SEQ ID NO: 70)
MVSSDSVNSRVETLAGSGISTIPKEYIRPKDELVNIGDIFEQEKNNEGPQVPTIDLKEIESD
NEKVRAKCREKLKKATVDWGVMHLVNHGISDELMDKVRKAGKAFFDLPIEQKEKYAN
DQASGKIQGYGSKLANNASGQLEWEDYFFHCVYPEDKRDLSIWPQTPADYIEATAEYA
KQLRELATKVLKVLSLGLGLDEGRLEKEVGGLEELLLQMKINYYPKCPQPELALGVEAH
TDVSALTFILHNMVPGLQLFYEGKWVTAKCVPNSIVMHIGDTLEILSNGKYKSILHRGM
VNKEKVRISWAVFCEPPKEKIILKPLPETVSEDEPAMFPPRTFAEHIQHKLFRKSQEALLP
K
Pyrus communis ANS
Amino acid sequence
(SEQ ID NO: 71)
MVSSDSVNSRVETLAGSGISTIPKEYIRPKDELVNIGDIFEQEKNNEGPQVPTIDLKEIESD
NEKVRAKCREELKKAAVDWGVMHLVNHGISDELMDKVRKAGKAFFDLPIEQKEKYAN
DQASGKIQGYGSKLANNASGQLEWEDYFFHCVYPEDKRDLSIWPQTPADYIEATAEYA
KQLRELATKVLKVLSLGLGLDEGRLEKEVGGLEELLLQMKINYYPKCPQPELALGVEAH
TDVSALTFILHNMVPGLQLFYEGKWVTAKCVPNSIVMHIGDTLEILSNGKYKSILHRGM
VNKEKVRISWAVFCEPPKEKIILKPLPETVSEDEPAMFPPRTFAEHIQHKLFRKSQEALLP
K
Prunus avium ANS
Amino acid sequence
(SEQ ID NO: 72)
MVSSDSVNSRVETLASSGIATIPKEYIRPKEELINIGDIFEQEKSTDGPQVPTIDLKEIDSEN
EKVRERCREELNKAAVDWGVMHLVNHGISDELMDRVRKAGKAFFDLPIEQKEKYAND
QASGKIQGYGSKLANNASGQLEWEDYFFHLIFPEDKRDLSIWPQTPADYIEATAEYAKE
LRALATKVLRVLSLGLGLEEGRLEKEVGGLEELLLQMKINYYPVCPQPELALGVEAHTD
VSALTFILHNMVPGLQLFYEGKWVTAKCVPNSIVMHIGDTIEILSNGKYKSILHRGMVN
KEKVRISWAVFCEPPKEKIILKPLPETVSETEPPIFPPRTFAEHIQHKLFRKSQEALLNK
Fragaria x ananassa ANS
Amino acid sequence
(SEQ ID NO: 73)
MVTAASIGSRVESLASSGISTIPKEYVRPEEELVNIGDIFEDEKSTEGPQVPTIDLKEIDSED
IKVREKCREELKKAAIDWGVMHLVNHGISDELMERVKKAGKAFFDLPVEQKEKYAND
QASGKIQGYGSKLANNASGQLEWEDYFFHCVYPEDKRDLSIWPQTPSDYIVATSEYAKE
LRGLTTKILSILSLGLGLEEGRLEKEVGGLEELLLQMKINYYPKCPQPELALGVEAHTDIS
ALTFILHNMVPGLQLFYGGKWVTAKCVPNSVVMHIGDTLEILSNGKYKSILHRGLVNKE
KVRISWAVFCEPPKEKIILKPLPETVSEEEPAIFPPRTFFEHIQHKLFRQSQEALVSTKESAA
LKSTKESALKSTKEAALISTN
Vitis vinifera ANS
Amino acid sequence
(SEQ ID NO: 74)
MVTSVAPRVESLSSSGIQSIPKEYIRPQEELTSIGNVFEEEKKDEGPQVPTIDLKDIESEDE
VVRERCREELKKAAMEWGVMHLVNHGISDDLINRVKVAGETFFNLPMEEKEKYANDQ
ASGKIAGYGSKLANNASGQLEWEDYFFHLIFPEDKRDMTIWPKTPSDYVPATCEYSVKL
RSLATKILSVLSLGLGLEEGRLEKEVGGMEELLLQKKINYYPKCPQPELALGVEAHTDVS
ALTFILHNMVPGLQLFYEGKWVTAKCVPNSIIMHIGDTIEILSNGKYKSILHRGLVNKEK
VRISWAVFCEPPKEKIILKPLPETVSETEPPLFPPRTFSQHIQHKLFRKTQEALLSK
Ipomoea purpurea anthocyanidin synthase (ANS)
Amino acid sequence
(SEQ ID NO: 75)
MLSTITATVPSRVERLAGSGIERIPKEYIRPEEERRSIGDIFEEEKIAGGPQVPTVDLKGINS
EDLEVREKCREELRKAAVDWGVMHLVNHGIPEELTGRVKAAGEGFFGQPIEEKEKYAN
DQAAGNVQGYGSKLANNASGQLEWEDYFFHCIFPEDKTDLSIWPKTPSDYIDATREYAK
QLRALATKVLAVLSLGLGLEEGRLEKEVGGMEELLLQMKINYYPKCPQPELALGVEAH
TDVSALTFILHNMVPGLQLFYGGKWVTAKCVPNSIIMHVGDTVEILSNGKYKSILHRGV
VNREKVRVSWAVFCEPPKDKILLQPLPETVSEAEPPRFPPRTFAQHIKHKLFRQSDQEAA
HTPKPDNDDDHQSN
Camellia sinensis ANS
Amino acid sequence
(SEQ ID NO: 76)
MTTVAAPRVQSLATSGIESIPKEYVRPKEELTGIGNIFEEEKNEEGPQVPTIDLKDIDSEVE
EVRERCREALKKAAVDWGVMHLVNHGIADDVRERVKVAGEGFFEQPVEEKEKYANDP
DNGNLQGYGSKLANNACGQFEWEDYFFHLAYPEDKCDMSIWPKTPTDYIPATVEYAKQ
LRALATKTLSILSLGLGLEENKLEKEVGGKEELLLQMKINYYPKCPQPELALGVEAHTDL
SAVSFILPSMVPGLQLFYEGKWITAKCVPNSIIMLIGDTVEILSNGKYKSILHRGLVNKEK
VRISWAVFCEPPKEKIILKPLPETVSEAEPPLEPPRTFAQHIQHKLFRKSQELGSK
Citrus sinensis anthocyanidin synthase (ANS)
Amino acid sequence
(SEQ ID NO: 77)
MVTPTARRVESLARSGIQAIPKEYVRPKEELMGIGNIFEEEEKDEGPQVPTIDLKEIDSED
RVEREKCREELKKAAMDWGVMHLVNHGISDDLTERVKRAGQAFFDQPVEEKEKYANE
QASGKIQGYGSKLANNASGQLEWEDYFFHLIYPEDKRDMSIWPKTPSDYTEATSEYARQ
LRSLATKILAVLSLGLGLEEGRLEKEVGGLEELLLQMKINYYPKCPQPELALGVEAHTD
VSALTFILHNMVPGLQLFYKDKWVTAKCVPNSIILHIGDTIEILSNGEYKSILHRGLVNKE
KVRISWAVFCEPPKDKIILKPLPETVSEQKPAMFPPRTFQQHIEHKLFRRTQDALLSDEE
Vaccinium ashei ANS
Amino acid sequence
(SEQ ID NO: 78)
MVSTMVAAPSR VESLASSGIQSIPKEYVRPKEELTSIGNIFEEEKKHEGPQVPTIDLEDLVS
EDKEARERCHEALKKAATEWGVMHLVNHGVPEELMDRVRVAGEGFENQPVEEKEKY
ANDHDTGNSGKIQGYGSKLANNASGQLEWEDYFFHTVYPEDKRDMKIWPKNPSDYIPA
TSEYANHLRALTTKVLSALSVCLGLEEDRLEKEVGGKDELVIQMKINYYPKCPQPELAL
GVEAHTDVSALTFILHNMVPGLQLFYEGKWITAKCVPNSIIMHIGDTVEILSNGKYKSIL
HRGLVNKEKVRISWAAFCEPPKEKIILKPLPETVSETEPARYPPRTFSQHIEHKLFRKTQA
LNGA
Populus trichocarpa ANS
Amino acid sequence
(SEQ ID NO: 79)
MMVTSSFVVPRVESLASSGIQSIPKEYIRPQEELSSIRDVFEEEKKVEGPQVPTIDLKEMES
EDKVVREKCREELVKAATEWGVMHLVNHGIPDDLIDRVKKAGQAFFDLPIEEKEKHAN
DQASGNVQGYGSKLANNASGQLEWEDYFFHLIFPEDKRDFSIWPKTPSDYTEVTSEYAR
QLRSLATKILSVLSLGLGLEEGRLEKEVGGLEELLLQMKINYYPKCPQPDLALGVEAHSD
VSALTFILHNMVPGLQLLYEGKWITAKCVPNSIIMHIGDTVEILSNGKYKSIIHRGLVNKE
KVRISWAVFCEPPKAKIILKPLAEIVTEAEPPLFPPRTFSQHIEHKLFRKTQDSLLPRKAN
Rhodobacter capsulatus TAL
Amino acid sequence
(SEQ ID NO: 80)
MLDATIGRKRMTLQSQTAKDCLALDGALTLVQCEAIATHRSRISVTPALRERCARAHAR
LEHAIAEQRHIYGITTGEGPLANRLIGADQGAELQQNLIYHLATGVGPKLSWAEARALM
LARLNSILQGASGASPETIDRIVAVLNAGFAPEVPAQGTVGASGDLTPLAHMVLALQGR
GRMIDPSGRVQEAGAVMDRLCGGPLTLAARDGLALVNGTSAMTAIAALTGVEAARAID
AALRHSAVLMEVLSGHAEAWHPAFAELRPHPGQLRATERLAQALDGAGRVCRTLTAA
RRLTAADLRPEDHPAQDAYSLRVVPQLVGAVWDTLDWHDRVVTCELNSVTDNPIFPEG
CAVPALHGGNFMGVHVALASDALNAALVTLAGLVERQIARLTDEKLNKGLPAFLHGG
QAGLQSGFMGAQVTATALLAEMRANATPVSVQSLSTNGANQDVVSMGTIAARRARAQ
LLPLSQIQAILALALAQAMDLLDDPEGQAGWSLTARDLRDRIRAVSPGLRADRPLAGHIE
AVAQGLRHPSAAADPPA
Rice TAL
Amino acid sequence
(SEQ ID NO: 81)
MAGNGPINKEDPLNWGAAAAEMAGSHLDEVKRMVAQFREPLVKIQGATLRVGQVAA
VAQAKDAARVAVELDEEARPRVKASSEWILTCIAHGGDIYGVTTGFGGTSHRRTKDGP
ALQVELLRYLNAGIFGTGSDGHTLPSETVRAAMLVRINTLLQGYSGIRFEILEAITKLLNT
GVTPCLPLRGTITASGDLVPLSYIAGLITGRPNAQAISPDGRKVDAAEAFKLAGIEGGFFT
LNPKEGLAIVNGTSVGSALAATVMFDANILAVLSEVLSAVFCEVMNGKPEYTDHLTHKL
KHHPGSIDAAAIMEHILAGSSFMSHAKKVNEMDPLLKPKQDRYALRTSPQWLGPQIQVI
RAATKSIEREVNSVNDNPVIDVHRGKALHGGNFQGTPIGVSMDNARLAIANIGKLMFAQ
FSELVNEFYNNGLTSNLAGSRNPSLDYGFKGTEIAMASYSSELQYLANPITNHVQSAEQH
NQDVNSLGLVSARKTLEAVDILKLMTSTYIVALCQAVDLRHLEENIKSSVKNCVTQVAK
KVLTMNPTGDLSSARFSEKNLLTAIDREAVFSYADDPCSANYPLMQKLRAVLVEHALTS
GDRRARGLRVLQDHQVRGGAPLCAAPGDRGRPRRRRQRTAPVANRIVESRSFPLYRFV
REELGCVFLTGEKLKSPGEECNKVFLGISQGKLIDPMLDCLKEWNGEPLPIN
Parsley TAL
Amino acid sequence
(SEQ ID NO: 82)
FLNAGIFGNGSDNTLPHSATRAAMLVRINTLLQGYSGIRFEILEAITKFLNQNITPCLPLRG
TITASGDLVPLSYIAGLLTGRPNSKAVGPTGVILSPEEAFKLAGVEGGFFELQPKEGLALV
NGTAVGSGMASMVLFEANILAVLAEVMSAIFAEVMQGKPEFTDHLTHKLKHHPGQIEA
AAIMEHILDGSAYVKAAQKLHEMDPLQKPKQDRYALRTSPQWLGPQIEVIRSSTKMIER
EINSVNDNPLIDVSRNKAIHGGNFQGTPIGVSMDNTRLAIAAIGKLMFAQFSELVNDFYN
NGLPSNLSGGRNPSLDYGFKGAEIAMASYCSELQFLANPVTNHVQSAEQHNQDVNSLG
LISSRKTSEAVEILKLMSTTFLVGLCQAIDLRHLEENLKSTVKNTVSSVAKRVLTMGVNG
ELHPSRFCEKDLLRVVDREYIFAYIDDPCSATYPLMQKLRQTLVEHALKNGDNERNLST
SIFQKIATFEDELKALLPKEVESARAALESGNPAIPNRIEECRSYPLYKFVRKELGTEYLT
GEKVTSPGEEFEKVFIAMSKGEIIDPLLECLESWNGAPLPIC
Tomato TAL
Amino acid sequence
(SEQ ID NO: 83)
MDLCKKSINDPLNWEMAADSLRGSHLDEVKKMVDEFRKPIVKLGGETLSVAQVASIAN
VDDKSNGVKVELSESARAGVKASSDWVMDSMSKGTDSYGVTAGFGATSHRRTKNGG
ALQKELIRFLNAGVFGNGIESFHTLPHSATRAAMLVRINTLLQGYSGIRFEILEAITKLINS
NITPCLPLRGTITASGDLVPLSYIAGLLTGRPNSKAVGPNGEKLNAEEAFCVAGISGGFFE
LQPKEGLALVNGTAVGSAMASIVLFESNIFAVMSEVLSAIFTEVMNGKPEFTDYLTHKL
KHHPGQIEAAAIMEHILDGSSYVKVAQKLHEMDPLQKPKQDRYALRTSPQWLGPQIEVI
RAATKMIEREINSVNDNPLIDVSRNKALHGGNFQGTPIGVSMDNTRLALASIGKLMFAQ
FSELVNDYYNNGLPSNLTAGRNPSLDYGFKGAEIAMASYCSELQFLANPVTNHVQSAEQ
HNQDVNSLGLISARKTAKAVDILKIMSSTYLVALCQAIDLRHLEENLKSVVKNTVSQVA
KRTLTMGANGELHPARFSEKELLRVVDREYLFAYADDPCSSNYPLMQKLRQVLVDQA
MKNGESEKNVNSSIFQKIGAFEDELIAVLPKEVESVRAVFESGNPLIRNRITECRSYPLYR
LVREELGTELLTGEKVRSPGEEIDKVFTAICNGQIIDPLLECLKSWNGAPLPIC
Arabidopsis TAL
Amino acid sequence
(SEQ ID NO: 84)
MEINGAHKSNGGGVDAMLCGGDIKTKNMVINAEDPLNWGAAAEQMKGSHLDEVKRM
VAEFRKPVVNLGGETLTIGQVAAISTIGNSVKVELSETARAGVNASSDWVMESMNKGT
DSYGVTTGFGATSHRRTKNGVALQKELIRFLNAGIFGSTKETSHTLPHSATRAAMLVRIN
TLLQGFSGIRFEILEAITSFLNNNITPSLPLRGTITASGDLVPLSYIAGLLTGRPNSKATGPN
GEALTAEEAFKLAGISSGFFDLQPKEGLALVNGTAVGSGMASMVLFETNVLSVLAEILS
AVFAEVMSGKPEFTDHLTHRLKHHPGQIEAAAVMEHILDGSSYMKLAQKLHEMDPLQK
PKQDRYALRTSPQWLGPQIEVIRYATKSIEREINSVNDNPLIDVSRNKAIHGGNFQGTPIG
VSMDNTRLAIRAIGKLMFAQFSELVNDFYNNGLPSNLTASRNPSLDYGFKGAEIAMASY
CSELQYLANPVTSHVQSAEQHNQDVNSLGLISSRKTSEAVDILKLMSTTFLVAICQAVDL
RHLEENLRQTVKNTVSQVAKKVLTTGVNGELHPSRFCEKDLLKVVDREQVYTYADDPC
SATYPLIQKLRQVIVDHALVNGESEKNAVTSIFHKIGAFEEELKAVLPKEVEAARAAYDN
GTSAIPNRIKECRSYPLYRFVREELGTELLTGEKVTSPGEEFDKVFTAICEGKIIDPMMEC
LNEWNGAPIPIC

While only certain features of the invention have been illustrated and described herein, many modifications and changes will occur to those skilled in the art. It is, therefore, to be understood that the appended claims are intended to cover all such modifications and changes as falling within the true spirit of the invention.

Throughout this application, various references are referred to. The disclosures of these publications in their entireties are hereby incorporated by reference as if written herein.

What is claimed is:

1. A TAL module cell, wherein the TAL module cell is a microbial cell comprising an exogenous gene encoding for a tyrosine ammonia lyase (TAL), wherein the exogenous gene encoding for TAL encodes a polypeptide having at least 85% amino acid identity with the amino acid sequence of Rhodotorula glutinis tyrosine ammonia lyase (RgTAL) of SEQ ID NO: 29, Rhodobacter capsulatus TAL of SEQ ID NO: 80, Rice TAL of SEQ ID NO: 81, Parsley TAL of SEQ ID NO: 82, Tomato TAL of SEQ ID NO: 83, Arabidopsis TAL of SEQ ID NO: 84, or a combination thereof, having TAL activity.

2. The TAL module cell of claim 1, wherein a host cell for the TAL module cell is E. coli rpoA14(DE3).
